(19) United States
(12) Patent Application Publication
(10) Pub. No.: US 2025/0270318 A1
(43) Pub. Date: Aug. 28, 2025

(54) MULTISPECIFIC ANTIBODIES AND USES THEREOF

(71) Applicant: Ab Therapeutics, Inc., Hayward, CA (US)

(72) Inventors: Cai Huang, Hayward, CA (US); Su Huang, Hayward, CA (US); Riyao Yang, Hayward, CA (US); Yue Liu, Hayward, CA (US)

(21) Appl. No.: 18/849,230

(22) PCT Filed: Mar. 21, 2023
(86) PCT No.: PCT/US2023/015792

§ 371 Date: Sep. 20, 2024

Related U.S. Application Data
(60) Provisional application No. 63/322,163, filed on Mar. 21, 2022.

(51) Int. Cl. C07K 16/28; A61K 39/00; A61K 47/68; A61P 35/00

(52) U.S. Cl. CPC C07K 16/2803; A61K 47/6879; A61P 35/00; C07K 16/2827; A61K 2039/505; C07K 2317/14; C07K 2317/31; C07K 2317/52; C07K 2317/565; C07K 2317/569; C07K 2317/73

(57) ABSTRACT

This disclosure relates to multispecific antibodies (e.g., bispecific antibodies or tri-specific antibodies) or antigen-binding fragments thereof. In one aspect, the multispecific antibodies or antigen-binding fragments thereof binds to TIGIT, LAG3, and/or PD-L1, or a combination thereof.

### CLAIM OF PRIORITY

This application claims the benefit of U.S. Provisional Application App. No. 63/322,163, filed on Mar. 21, 2022. The entire contents of the foregoing application are incorporated herein by reference.

### TECHNICAL FIELD

This disclosure relates to multispecific antibodies or antigen-binding fragments thereof.

### BACKGROUND

A multispecific antibody is an artificial protein that can simultaneously bind to two or more different epitopes. This opens up a wide range of applications, including redirecting T cells to tumor cells, blocking two different signaling pathways simultaneously, dual targeting of different disease mediators, and delivering payloads to targeted sites. The approval of catumaxomab (anti-EpCAM and anti-CD3) and blinatumomab (anti-CD19 and anti-CD3) has become a major milestone in the development of multispecific antibodies.

As multispecific antibodies have various applications, there is a need to continue to develop various therapeutics based on multispecific antibodies.

### SUMMARY

The landscape of current cancer immunotherapy is dominated by antibodies targeting PD-1/PD-L1 and CTLA-4 that have transformed cancer therapy, yet their efficacy is limited by primary and acquired resistance. The blockade of additional immune checkpoints, especially TIGIT and LAG-3, has been extensively explored, but so far only a LAG-3 antibody has been approved for combination with nivolumab to treat unresectable or metastatic melanoma. Here we report the development of a PDL1×TIGIT bi-specific antibody (BsAb), a PDL1×LAG3 BsAb, and a PDL1×TIGIT×LAG3 tri-specific antibody (TsAb), all with intact Fc function. In in vitro cell-based assays, these antibodies promote greater T cell expansion and tumor cell killing than benchmark antibodies and antibody combinations in an Fc-dependent manner, likely by facilitating T cell interactions (bridging) with cancer cells and monocytes, in addition to blocking immune checkpoints. In animal models, PD-L1/TIGIT BsAb and PD-L1/LAG3/TIGIT TsAb outperformed benchmarks in tumor suppression. This study demonstrates the potential of a new generation of multispecific checkpoint inhibitors to overcome resistance to current monospecific checkpoint antibodies or their combinations for the treatment of human cancers.

This disclosure relates to antibodies or antigen-binding fragments, in some embodiments, the antibodies or antigen-binding fragments specifically bind to TIGIT, LAG3, PD-L1, or a combination thereof. In some embodiments, the disclosure relates to development of TIGIT/PD-L1 or LAG3/PD-L1 targeting bispecific antibodies. In some embodiments, the disclosure relates to development of PD-L1/LAG3/TIGIT targeting tri-specific antibodies.

In one aspect, the disclosure relates to an antibody or antigen-binding fragment thereof that binds to LAG3 (Lymphocyte-activation gene 3), comprising: a heavy-chain antibody variable domain (VHH) comprising complementarity determining regions (CDRs) 1, 2, and 3, in some embodiments, the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence. In some embodiments, the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively;
(4) the selected VHH CDRs 1, 2, 3 amino acid sequences are set forth in SEQ ID NOs: 10, 11, and 12, respectively; and
(5) the selected VHH CDRs 1, 2, 3 amino acid sequences are set forth in SEQ ID NOs: 13, 14, and 15, respectively;

In some embodiments, the VHH comprises CDRs 1, 2, 3 with the amino acid sequences set forth in SEQ ID NOs: 1, 2, and 3, respectively. In some embodiments, the VHH comprises CDRs 1, 2, 3 with the amino acid sequences set forth in SEQ ID NOs: 4, 5, and 6, respectively. In some embodiments, the VHH comprises CDRs 1, 2, 3 with the amino acid sequences set forth in SEQ ID NOs: 7, 8, and 9, respectively. In some embodiments, the VHH comprises CDRs 1, 2, 3 with the amino acid sequences set forth in SEQ ID NOs: 10, 11, and 12, respectively. In some embodiments, the VHH comprises CDRs 1, 2, 3 with the amino acid sequences set forth in SEQ ID NOs: 13, 14, and 15, respectively.

In one aspect, the disclosure relates to an antibody or antigen-binding fragment thereof that binds to LAG3 comprising a heavy-chain antibody variable domain (VHH) comprising an amino acid sequence that is at least 80% identical to a selected VHH sequence. In some embodiments, the selected VHH sequence is selected from the group consisting of SEQ ID NOs: 22-26.

In some embodiments, the antibody or antigen-binding fragment specifically binds to human LAG3.

In one aspect, the disclosure relates to an antibody or antigen-binding fragment thereof that binds to TIGIT (T cell immunoreceptor with Ig and ITIM domains), comprising: a heavy-chain antibody variable domain (VHH) comprising complementarity determining regions (CDRs) 1, 2, and 3, in some embodiments, the VHH CDR1 region comprises an amino acid sequence that is at least 80% identical to SEQ ID NO: 16, the VHH CDR2 region comprises an amino acid sequence that is at least 80% identical to SEQ ID NO: 17, and the VHH CDR3 region comprises an amino acid sequence that is at least 80% identical to SEQ ID NO: 18. In some embodiments, the VHH comprises CDRs 1, 2, 3 with the amino acid sequences set forth in SEQ ID NOs: 16, 17, and 18, respectively.

In one aspect, the disclosure relates to an antibody or antigen-binding fragment thereof that binds to TIGIT comprising a heavy-chain antibody variable domain (VHH) comprising an amino acid sequence that is at least 80% identical to SEQ ID NO: 27.

In some embodiments, the antibody or antigen-binding fragment specifically binds to human TIGIT.

In one aspect, the disclosure relates to an antibody or antigen-binding fragment thereof that binds to PD-L1 (Programmed death-ligand 1), comprising: a heavy-chain antibody variable domain (VHH) comprising complementarity determining regions (CDRs) 1, 2, and 3, in some embodiments, the VHH CDR1 region comprises an amino acid sequence that is at least 80% identical to SEQ ID NO: 19, the VHH CDR2 region comprises an amino acid sequence that is at least 80% identical to SEQ ID NO: 20, and the VHH CDR3 region comprises an amino acid sequence that is at least 80% identical to SEQ ID NO: 21. In some embodiments, the VHH comprises CDRs 1, 2, 3 with the amino acid sequences set forth in SEQ ID NOs: 19, 20, and 21, respectively.

In one aspect, the disclosure relates to an antibody or antigen-binding fragment thereof that binds to PD-L1 comprising a heavy-chain antibody variable domain (VHH) comprising an amino acid sequence that is at least 80% identical to SEQ ID NO: 28.

In some embodiments, the antibody or antigen-binding fragment specifically binds to human PD-L1. In some embodiments, the antibody or antigen-binding fragment is a humanized antibody or antigen-binding fragment thereof.

In one aspect, the disclosure relates to an antibody or antigen-binding fragment thereof comprising the VHH CDRs 1, 2, 3, of the antibody or antigen-binding fragment thereof as described herein.

In some embodiments, the antibody or antigen-binding fragment comprises a human IgG Fc (e.g., a human IgG Fc comprising an amino acid sequence that is at least 80% identical to SEQ ID NO: 29).

In some embodiments, the antibody or antigen-binding fragment comprises two or more heavy-chain antibody variable domains.

In one aspect, the disclosure relates to an antibody or antigen-binding fragment thereof that cross-competes with the antibody or antigen-binding fragment thereof as described herein.

In some embodiments, the antibody or antigen-binding fragment is a single-chain variable fragment (scFv) or a multispecific antibody (e.g., a bispecific antibody).

In one aspect, the disclosure relates to a multi-specific antibody or antigen-binding fragment thereof, comprising a first antigen-binding site that specifically binds to PD-L1, and a second antigen-binding site that specifically binds to TIGIT. In some embodiments, the first antigen-binding site comprises a first heavy-chain antibody variable domain (VHH1) that specifically binds to PD-L1, in some embodiments, the VHH1 comprises complementarity determining regions (CDRs) 1, 2, and 3, in some embodiments, the VHH1 CDR1 region comprises an amino acid sequence that is at least 80% identical to SEQ ID NO: 19, the VHH1 CDR2 region comprises an amino acid sequence that is at least 80% identical to SEQ ID NO: 20, and the VHH1 CDR3 region comprises an amino acid sequence that is at least 80% identical to SEQ ID NO: 21. In some embodiments, the VHH1 comprises CDRs 1, 2, 3 with the amino acid sequences set forth in SEQ ID NOs: 19, 20, and 21, respectively. In some embodiments, the VHH1 comprises an amino acid sequence that is at least 80% identical to SEQ ID NO: 28.

In some embodiments, the second antigen-binding site specifically binds to TIGIT, and the second antigen-binding site comprises a second heavy-chain antibody variable domain (VHH2) that specifically binds to TIGIT. In some embodiments, the VHH2 comprises complementarity determining regions (CDRs) 1, 2, and 3, in some embodiments, the VHH2 CDR1 region comprises an amino acid sequence that is at least 80% identical to SEQ ID NO: 16, the VHH2 CDR2 region comprises an amino acid sequence that is at least 80% identical to SEQ ID NO: 17, and the VHH2 CDR3 region comprises an amino acid sequence that is at least 80% identical to SEQ ID NO: 18. In some embodiments, the VHH2 comprises an amino acid sequence that is at least 80% identical to SEQ ID NO: 27.

In some embodiments, the multi-specific antibody or antigen-binding fragment thereof as described herein, further comprises a third antigen-binding site that specifically binds to LAG3. In some embodiments, the third antigen-binding site specifically binds to LAG3, and the third antigen-binding site comprises a third heavy-chain antibody variable domain (VHH3) comprising complementarity determining regions (CDRs) 1, 2, and 3, in some embodiments, the VHH3 CDR1 region comprises an amino acid sequence that is at least 80% identical to a selected VHH3 CDR1 amino acid sequence, the VHH3 CDR2 region comprises an amino acid sequence that is at least 80% identical to a selected VHH3 CDR2 amino acid sequence, and the VHH3 CDR3 region comprises an amino acid sequence that is at least 80% identical to a selected VHH3 CDR3 amino acid sequence. In some embodiments, the selected VHH3 CDRs 1, 2, and 3 amino acid sequences are one of the following:
(1) the selected VHH3 CDRs 1, 2, 3 amino acid sequences are set forth in SEQ ID NOs: 1, 2, and 3, respectively;
(2) the selected VHH3 CDRs 1, 2, 3 amino acid sequences are set forth in SEQ ID NOs: 4, 5, and 6, respectively;
(3) the selected VHH3 CDRs 1, 2, 3 amino acid sequences are set forth in SEQ ID NOs: 7, 8, and 9, respectively;
(4) the selected VHH3 CDRs 1, 2, 3 amino acid sequences are set forth in SEQ ID NOs: 10, 11, and 12, respectively; and
(5) the selected VHH3 CDRs 1, 2, 3 amino acid sequences are set forth in SEQ ID NOs: 13, 14, and 15, respectively.

In some embodiments, the VHH3 comprises an amino acid sequence that is at least 80% identical to a selected VHH3 sequence. In some embodiments, the selected VHH3 sequence is selected from the group consisting of SEQ ID NOs: 22-26.

In one aspect, the disclosure relates to a multi-specific antibody or antigen-binding fragment thereof, comprising a first antigen-binding site that specifically binds to PD-L1, and a second antigen-binding site that specifically binds to LAG3. In some embodiments, the first antigen-binding site comprises a first heavy-chain antibody variable domain (VHH1) that specifically binds to PD-L1, in some embodiments, the VHH1 comprises complementarity determining regions (CDRs) 1, 2, and 3. In some embodiments, the VHH1 CDR1 region comprises an amino acid sequence that is at least 80% identical to SEQ ID NO: 19, the VHH1 CDR2 region comprises an amino acid sequence that is at least 80% identical to SEQ ID NO: 20, and the VHH1 CDR3 region comprises an amino acid sequence that is at least 80% identical to SEQ ID NO: 21. In some embodiments, the VHH1 comprises CDRs 1, 2, 3 with the amino acid sequences set forth in SEQ ID NOs: 19, 20, and 21, respectively. In some embodiments, the VHH1 comprises an amino acid sequence that is at least 80% identical to SEQ ID NO: 28.

In some embodiments, the second antigen-binding site specifically binds to LAG3, and the second antigen-binding site comprises a second heavy-chain antibody variable domain (VHH2) comprising complementarity determining regions (CDRs) 1, 2, and 3. In some embodiments, the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence. In some embodiments, the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively;
(4) the selected VHH2 CDRs 1, 2, 3 amino acid sequences are set forth in SEQ ID NOs: 10, 11, and 12, respectively; and
(5) the selected VHH2 CDRs 1, 2, 3 amino acid sequences are set forth in SEQ ID NOs: 13, 14, and 15, respectively.

In some embodiments, the VHH2 comprises an amino acid sequence that is at least 80% identical to a selected VHH2 sequence. In some embodiments, the selected VHH2 sequence is selected from the group consisting of SEQ ID NOs: 22-26.

In some embodiments, the multi-specific antibody or antigen-binding fragment thereof as described herein, further comprises a third antigen-binding site that specifically binds to TIGIT. In some embodiments, the third antigen-binding site specifically binds to TIGIT, and the third antigen-binding site comprises a third heavy-chain antibody variable domain (VHH3) that specifically binds to TIGIT. In some embodiments, the VHH3 comprises complementarity determining regions (CDRs) 1, 2, and 3. In some embodiments, the VHH3 CDR1 region comprises an amino acid sequence that is at least 80% identical to SEQ ID NO: 16, the VHH3 CDR2 region comprises an amino acid sequence that is at least 80% identical to SEQ ID NO: 17, and the VHH3 CDR3 region comprises an amino acid sequence that is at least 80% identical to SEQ ID NO: 18. In some embodiments, the VHH3 comprises an amino acid sequence that is at least 80% identical to SEQ ID NO: 27.

In some embodiments, the VHH1, VHH2, and/or VHH3 are linked by a linker peptide sequence. In some embodiments, the linker peptide sequence comprises a sequence that is at least 80% identical to any one of SEQ ID NOs: 30-35.

In one aspect, the disclosure relates to a polypeptide complex, comprising (a) a first polypeptide comprising from N-terminus to C-terminus: a first heavy-chain antibody variable domain (VHH1), a first linker peptide sequence, a second heavy-chain antibody variable domain (VHH2), and a first Fc region (optionally including a hinge region); and (b) a second polypeptide comprising from N-terminus to C-terminus: a third heavy-chain antibody variable domain (VHH3), a second linker peptide sequence, a fourth heavy-chain antibody variable domain (VHH4), and a second Fc region (optionally including a hinge region). In some embodiments, the VHH1 and VHH3 specifically bind to TIGIT. In some embodiments, the VHH2 and VHH4 specifically bind to PD-L. In some embodiments, the first Fc region and/or the second Fc region comprise a sequence that is at least 80% identical to SEQ ID NO: 29. In some embodiments, the VHH1 and/or VHH3 comprise an amino acid sequence that is at least 90% identical to SEQ ID NO: 27. In some embodiments, the VHH2 and/or VHH4 comprise an amino acid sequence that is at least 90% identical to SEQ ID NO: 28. In some embodiments, the first polypeptide further comprises a fifth heavy-chain antibody variable domain (VHH5) that specifically binds to LAG3. In some embodiments, the VHH5 is linked to the N-terminus of the VHH1 via a third linker peptide sequence. In some embodiments, the VHH5 comprises an amino acid sequence that is at least 90% identical to SEQ ID NO: 22, 23, 24, 25, or 26. In some embodiments, the first, second, and/or third linker peptide sequence comprises a sequence that is at least 80% identical to any one of SEQ ID NOs: 30-35.

In one aspect, the disclosure relates to a polypeptide complex, comprising (a) a first polypeptide comprising from N-terminus to C-terminus: a first heavy-chain antibody variable domain (VHH1), a first linker peptide sequence, a second heavy-chain antibody variable domain (VHH2), and a first Fc region (optionally including a hinge region); and (b) a second polypeptide comprising from N-terminus to C-terminus: a third heavy-chain antibody variable domain (VHH3), a second linker peptide sequence, a fourth heavy-chain antibody variable domain (VHH4), and a second Fc region (optionally including a hinge region). In some embodiments, the VHH1 and VHH3 specifically bind to LAG3. In some embodiments, the VHH2 and VHH4 specifically bind to PD-L1. In some embodiments, the first Fc region and/or the second Fc region comprise a sequence that is at least 80% identical to SEQ ID NO: 29. In some embodiments, the VHH1 and/or VHH3 comprise an amino acid sequence that is at least 90% identical to SEQ ID NO: 22, 23, 24, 25, or 26. In some embodiments, the VHH2 and/or VHH4 comprise an amino acid sequence that is at least 90% identical to SEQ ID NO: 28. In some embodiments, the first polypeptide further comprises a fifth heavy-chain antibody variable domain (VHH5) that specifically binds to TIGIT. In some embodiments, the VHH5 is linked to the N-terminus of the VHH1 via a third linker peptide sequence. In some embodiments, the VHH5 comprises an amino acid sequence that is at least 90% identical to SEQ ID NO: 27. In some embodiments, the first, second, and/or third linker peptide sequence comprises a sequence that is at least 80% identical to any one of SEQ ID NOs: 30-35.

In one aspect, the disclosure relates to a nucleic acid comprising a polynucleotide encoding the antibody or antigen-binding fragment thereof as described herein, the multi-specific antibody or antigen-binding fragment thereof as described herein, or the polypeptide complex as described herein. In some embodiments, the nucleic acid is a DNA (e.g., cDNA) or RNA (e.g., mRNA).

In one aspect, the disclosure relates to a vector comprising one or more of the nucleic acids as described herein.

In one aspect, the disclosure relates to a cell comprising the vector as described herein. In some embodiments, the cell is a CHO cell.

In one aspect, the disclosure relates to a cell comprising one or more of the nucleic acid as described herein.

In one aspect, the disclosure relates to a method of producing an antibody or an antigen-binding fragment thereof, the method comprising
(a) culturing the cell as described herein under conditions sufficient for the cell to produce the antibody or the antigen-binding fragment; and
(b) collecting the antibody or the antigen-binding fragment produced by the cell.

In one aspect, the disclosure relates to an antibody-drug conjugate comprising the antibody or antigen-binding fragment thereof as described herein, the multi-specific antibody or antigen-binding fragment thereof as described herein, or the polypeptide complex as described herein, covalently bound to a therapeutic agent. In some embodiments, the therapeutic agent is a cytotoxic or cytostatic agent.

In one aspect, the disclosure relates to a method of treating a subject having cancer, the method comprising administering a therapeutically effective amount of a composition comprising the antibody or antigen-binding fragment thereof as described herein, the multi-specific antibody or antigen-binding fragment thereof as described herein, the polypeptide complex as described herein, or the antibody-drug conjugate as described herein, to the subject.

In some embodiments, the subject has a cancer expressing CD155, MHC class II molecules and/or FGL1 (Fibrinogen-like protein 1).

In some embodiments, the subject has a cancer expressing PD-L1.

In some embodiments, the cancer is liver cancer melanoma, non-small cell lung cancer, head and neck squamous cell carcinoma, relapsed or refractory classical Hodgkin lymphoma, squamous cell lung cancer, renal cell carcinoma, pancreatic cancer, breast cancer, lung cancer, or cutaneous squamous cell carcinoma.

In one aspect, the disclosure relates to a method of decreasing the rate of tumor growth, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure relates to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure relates to a method of killing a tumor cell, the method comprising contacting a tumor cell with an effective amount of a composition comprising the antibody or antigen-binding fragment thereof as described herein, the multi-specific antibody or antigen-binding fragment thereof as described herein, the polypeptide complex as described herein, or the antibody-drug conjugate as described herein.

In one aspect, the disclosure relates to a pharmaceutical composition comprising the antibody or antigen-binding fragment thereof as described herein, the multi-specific antibody or antigen-binding fragment thereof as described herein, the polypeptide complex as described herein, and a pharmaceutically acceptable carrier.

In one aspect, the disclosure relates to a pharmaceutical composition comprising the antibody-drug conjugate as described herein, and a pharmaceutically acceptable carrier.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (VHH) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a VHH). Non-limiting examples of antibody fragments include, e.g., Fab, Fab′, F(ab′)₂, and Fv fragments, ScFv, and VHH.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to PD-L1 may be referred to as a PD-L1-specific antibody or an anti-PD-L1 antibody.

As used herein, the term “bispecific antibody” refers to an antibody that binds to two different epitopes. The epitopes can be on the same antigen or on different antigens.

As used herein, the term “trispecific antibody” refers to an antibody that binds to three different epitopes. The epitopes can be on the same antigen or on different antigens.

As used herein, the term “multispecific antibody” refers to an antibody that binds to two or more different epitopes. The epitopes can be on the same antigen or on different antigens. A multispecific antibody can be e.g., a bispecific antibody or a trispecific antibody. In some embodiments, the multispecific antibody binds to two, three, four, five, or six different epitopes.

As used herein, a “VHH” refers to the variable domain of a heavy chain antibody. In some embodiments, the VHH is a humanized VHH.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1G show target binding and blocking characteristics of PD-L1/TIGIT BsAb, PD-L1/LAG3 BsAb, and PD-L1/LAG3/TIGIT TsAb. A, Configuration of PD-L1/TIGIT BsAb, PD-L1/LAG3 BsAb, and PD-L1/LAG3/TIGIT TsAb. B-D, Target binding characteristics of PD-L1/TIGIT BsAb, PD-L1/LAG3 BsAb, and PD-L1/LAG3/TIGIT TsAb. Indicated target cells were incubated with PD-L1/TIGIT BsAb, PD-L1/LAG3 BsAb, PD-L1/LAG3/TIGIT TsAb, or control antibodies. Bound antibodies were then detected with a fluorescence-labeled secondary antibody specific for human IgG Fe by flow cytometry. The RKO colon cancer cell line expresses endogenous PD-L1. E-G, Target blocking characteristics of PD-L1/TIGIT BsAb, PD-L1/LAG3 BsAb, and PD-L1/LAG3/TIGIT TsAb. Indicated target cells were incubated with PD-L1/TIGIT BsAb, PD-L1/LAG3 BsAb, PD-L1/LAG3/TIGIT TsAb, or control antibodies, in the presence of biotinylated PD-1, LAG-3, or CD155. Bound biotinylated proteins were then detected with fluorescence-labeled streptavidin by flow cytometry. αTIGIT, αLAG, and αPDL1 represent parental in-house antibodies (bivalent monospecific VHH molecules expressed with an IgG1 Fc). Assays were performed in duplicates and data are presented as mean±SD.

FIGS. 2B-2E show PD-L1/TIGIT BsAb, PD-L1/LAG3 BsAb, and PD-L1/LAG3/TIGIT TsAb mediate interactions between T cells and cancer cells. A, Jurkat LAG-3 or TIGIT cells were labeled with ViaFluor 488, and incubated with RKO cells (PD-L1⁺CD155⁺) labeled with ViaFluor 405, in the presence of indicated antibodies (0.2 nM). Cell conjugates (events double-positive for the two fluorescent dyes. Boxed in red) were measured by flow cytometry. B and C, diagram showing the mechanism of PD-L1/TIGIT BsAb, PD-L1/LAG3 BsAb, and PD-L1/LAG3/TIGIT TsAb-mediated tumor cell/T cell bridging. D and E, dose-dependent bridging of cancer cells with T cells by PD-L1/TIGIT BsAb, PD-L1/LAG3 BsAb, and PD-L1/LAG3/TIGIT TsAb. Assays were performed in duplicates, and data are presented as mean±SD.

FIGS. 3A-3C show dose-dependent promotion of cancer cell killing and T cell expansion by PD-L1/TIGIT BsAb, PD-L1/LAG3 BsAb, and PD-L1/LAG3/TIGIT TsAb in comparison with their Fc-deficient counterparts or parental antibodies. RKO OKT3 cells were co-cultured with human PBMCs, in the presence of serial dilutions of indicated antibodies. LALA denotes L234A/L235A mutations that disable Fc effector function. αPDL1, αLAG3, and αTIGIT are parental monospecific antibodies used for the construction of PD-L1/TIGIT BsAb, PD-L1/LAG3 BsAb, and PD-L1/LAG3/TIGIT TsAb. The number of viable cancer cells (A), CD8 T cells (B), and CD4 T cells (C) was determined by flow cytometry. Gating strategy is shown in FIG. 7. Assays were performed in triplicate and data are presented as mean±SEM. Dash lines show 100% (IgG1 control). Statistical significance was assessed using two-way ANOVA with multiple comparisons.

FIGS. 4A-4C show dose-dependent promotion of cancer cell killing and T cell expansion by PD-L1/TIGIT BsAb, PD-L1/LAG3 BsAb, and PD-L1/LAG3/TIGIT TsAb. Raji PD-L1 CD155 OKT3 cells were co-cultured with human PBMCs, in the presence of serial dilutions of indicated antibodies. αPDL1, αLAG3, and αTIGIT are parental monospecific antibodies used for the construction of PD-L1/TIGIT BsAb (Fc+ and Fc−), PD-L1/LAG3 BsAb, and PD-L1/LAG3/TIGIT TsAb. Number of viable cancer cells (A), CD8 T cells (B), and CD4 T cells (C) was determined by flow cytometry. Gating strategy is shown in FIG. 7. Assays were performed in triplicate and data are presented as mean±SEM. Statistical significance was assessed using two-way ANOVA with multiple comparisons.

FIGS. 5A-5B show PD-L1/TIGIT BsAb, PD-L1/LAG3 BsAb, and PD-L1/LAG3/TIGIT TsAb efficacy is monocyte-dependent. Raji PD-L1 OKT3 cells were co-cultured with normal (A) or monocyte-depleted (B) PBMCs in the presence of indicated antibodies at 30 nM for 4 days. Viable cell count of CD8 T cells, CD4 T cells, and cancer cells were determined by flow cytometry. Statistical significance of test antibodies vs IgG1 control was assessed using one-way ANOVA with Fisher's LSD test for multiple comparisons and is shown by asterisks. *P<0.05; **P<0.01; **P<0.001; ****P<0.0001. ns, not significant.

FIGS. 6A-6C show PD-L1/TIGIT BsAb and PD-L1/LAG3/TIGIT TsAb efficiently suppress tumor growth in PBMC humanized animal model. A, Schematic of study design. NSG mice were co-engrafted with RKO OKT3 cells and human PBMCs (i.v.). When tumors reached a volume of approximately 100 mm³ (day 0), mice were treated with PBS or indicated antibodies (5 mg/kg, i.v.) and another dose of PBMCs. Antibody treatment was repeated on day 7, and the experiment was terminated on day 14. Tumor volume (B), animal body weight (C) were monitored during the treatment. Statistical significance on day 14 among treatment groups was assessed with two-tailed unpaired Student's t-test. *P<0.05; **P<0.01; **P<0.001; ****P<0.0001. ns, not significant. Data represent mean±SEM of four mice in each group.

FIG. 7 shows gating strategy for cancer cell/PBMC co-culture assays.

FIG. 8 shows monocyte depletion of PBMCs by adherence. Monocyte frequency in PBMCs with or without depletion was determined by flow cytometry after staining with CD14 antibodies.

FIG. 9A is a table summarizing anti-TIGIT antibody drugs in clinical development.

FIG. 9B shows assay systems described in the disclosure.

FIG. 9C shows schematic diagrams of T cells and cancer cells in the assay systems described in the disclosure.

FIGS. 10A-10B show absolute number of cancer cells when co-cultured with high monocyte content-PBMC-A and low monocyte content-PBMC-B, respectively.

FIGS. 11A-11C show T cell profiling of CD8+ and CD4+ cells in the presence of 100 nM, 33 nM, and 11 nM PBMC-A, respectively.

FIGS. 11D-11F show T cell profiling of CD8+ and CD4+ cells in the presence of 100 nM, 33 nM, and 11 nM PBMC-B, respectively.

FIGS. 12A-12C show absolute number of T cell proliferation in the presence of 100 nM, 33 nM, and 11 nM PBMC-A, respectively.

FIGS. 12D-12F show absolute number of T cell proliferation in the presence of 100 nM, 33 nM, and 11 nM PBMC-B, respectively.

FIG. 13 shows TIGIT expression spectrum in control cells, CD4+ T cells, Tregs, and CD8+ T cells.

FIG. 14 shows LAG3 expression level in CD8+ T cells, CD4+ nTreg cells, CD4+ iTreg cells, and CD4+ non-Treg cells.

FIG. 15 shows a schematic diagram of induction of T cell exhaustion.

FIGS. 16A-16C show percentage of PD-1, TIGIT, and LAG3 positive cells, respectively, during T cell exhaustion.

FIGS. 16D-16F show MFI values of PD-1, TIGIT, and LAG3, respectively, during T cell exhaustion.

FIGS. 17A-17C show numbers of viable cancer cells, CD8+ T cells, and CD4+ T cells, respectively, in the PBMC/exhausted T cell/cancer cell co-culture assays.

FIG. 17D shows IFN-γ levels of the PBMC/exhausted T cell/cancer cell co-culture assays as measured by ELISA.

FIG. 17E is a schematic diagram illustrating the interaction between cancer cells and T cells.

FIGS. 18A-18B show numbers of viable CD8+ and CD4+ T cells, respectively, in MLR assays including exhausted T cells and allogeneic DCs.

FIG. 19 shows a study design of an animal model to study T cell exhaustion.

FIGS. 20A-20B show tumor volumes of lung cancer and colon cancer development, respectively.

FIG. 21 is a set of flow cytometry results showing cancer and T cell numbers, and T cell exhausting status.

FIG. 22 shows a cartoon of hypothesized interactions between PD-L1/TIGIT BsAb and cells.

FIG. 23 shows a cartoon of hypothesized interactions between PD-L1/LAG3 BsAb and cells.

FIG. 24 shows a cartoon of hypothesized interactions between PD-L1/TIGIT BsAb and cells.

FIG. 25 is a schematic diagram showing PD-L1/TIGIT BsAb. The LAG3 or TIGIT binder is colored in red. the PD-L1 binder is colored in green.

FIG. 26 is a schematic diagram showing PD-L1/LAG3/TIGIT TsAb. The TIGIT binder is colored in purple. The LAG3 binder is colored in red. The PD-L1 binder is colored in green.

FIG. 27 lists CDR sequences of the VHHs from the anti-LAG3 antibodies described in the disclosure.

FIG. 28 lists CDR sequences of the VHHs from the anti-TIGIT antibodies described in the disclosure.

FIG. 29 lists CDR sequences of the VHHs from the anti-PD-L1 antibodies described in the disclosure.

FIG. 30 lists amino acid sequences of VHHs that specifically bind to LAG3, TIGIT, or PD-L1 as described in the disclosure.

FIG. 31 lists sequences discussed in the disclosure.

FIG. 32 shows IgG subclass and Fc status of therapeutic antibodies used in the disclosure.

FIG. 33 shows other antibodies/reagents used in the disclosure.

FIG. 34 lists primers used in the disclosure.

### DETAILED DESCRIPTION

Multiple checkpoint inhibitory receptors have been found on the surface of various immune cells. These receptors deliver inhibitory signals to suppress the function of host immune cells. Current researches indicate that most tumor cells hijack this pathway to evade the surveillance and attack of host immune system by inhibiting the activities of immune cells. PD-1 (the programmed cell death protein 1), a checkpoint receptor on activated T cells, after it binds to its ligand PD-L1 (the programmed death-ligand 1) on tumor cells, can stop T cells from attacking the tumor cells. Blocking the binding of PD-L1 to PD-1 can enhance T cells cytotoxicity against tumors. Several PD-1 and PD-L1 blocking antibodies have been approved by FDA. Successful cases of PD-1/PD-L1 monotherapy have also been reported. But the low response rate and adverse effects limit the use of the monotherapy. Of note, the combination of dual blockade of different checkpoint receptors produced encouraging effects on improving response rate and long term survival, indicating the engagement of multiple checkpoint receptors in regulating T cells function.

TIGIT (T cell immunoreceptor with Ig and ITIM domains) emerges as a new target for immune therapy. TIGIT (T cell immunoreceptor with Ig and ITIM domain), a checkpoint inhibitory receptor expressed on activated T cells, NK cells and Treg, specifically binds to CD155 expressed on most tumor cells to transfer inhibitory signals. TIGIT blockage can enhance NK cell killing activities and CD8+ T cell effector function, thus boosting anti-tumor immunity and reducing tumor burden. Several TIGIT blocking antibodies are currently in clinical trials. However, the monotherapy of either TIGIT or PD-L1 antibodies can only slightly inhibit tumor growth.

LAG3 (Lymphocyte-activation gene 3) belongs to immunoglobulin (Ig) superfamily, which is a 503-amino acid type I transmembrane protein with four extracellular Ig-like domains. LAG-3 is expressed on activated T cells, natural killer cells, B cells and/or plasmacytoid dendritic cells. LAG3 binds to its ligand MHC class II, negatively regulating T cell proliferation, activation, and homeostasis. Several LAG3 blocking antibodies are currently in clinical trials. Anti-LAG3 monoclonal antibodies can be used in combination with anti-PD-L1 antibodies to simultaneously block the interaction between LAG3/MHC class II and PD-L1/PD1, respectively. However, the monotherapy of either LAG3 or PD-L1 antibodies can only moderately inhibits tumor growth.

Bispecific antibodies, which bind to two targets simultaneously, can combine multiple functions of individual monoclonal antibodies (such as direct cancer cell lysis, blocking malignant signaling pathways, independent T cell activation), entailing a comparatively simpler treatment regimen than one requiring the combination of multiple separate agents. Moreover, bispecific antibodies can induce their target downregulation or shedding, which were not observed with the combination of two different antibodies. The dual blockage of TIGIT and PD-L1 can further improve overall response rate. It is also contemplated that, the PD-L1×TIGIT multispecific antibody can not only bind targets as a monoclonal antibody, but also bridge two target cells close together, leading to the clustering of the antibody-antigen complex between the interface of cells. The clustering can further increase antibody binding ability.

The present disclosure provides anti-PD-L1 antibodies and antigen binding fragments thereof, anti-TIGIT antibodies and antigen binding fragments thereof, anti-LAG3 antibodies and antigen binding fragments thereof, and multispecific antibodies that target PD-L1, TIGIT, and/or LAG3 and antigen binding fragments thereof.

### Introduction

Checkpoint inhibitors reactivate T cells by blockade checkpoint receptor/ligand interactions that restrict T cell activity. Several checkpoint inhibitor antibodies targeting CTLA-4 and PD-1/PD-L1 have been approved and have shown efficacy for a variety of tumor indications. Responders to such agents often exhibit durable remission, which is rarely seen for other therapies. However, many cancer patients do not benefit from such treatments due to primary or acquired resistance.

The landscape of checkpoint inhibitors is still dominated by antibodies that target PD-1 or PD-L1, although the potential of targeting other immune checkpoints are also being actively investigated. Among those TIGIT and LAG-3 are two distinct immune checkpoints that contribute to T cell exhaustion. TIGIT is expressed in T cells and NK cells, and inhibits T cell activation mainly by competing for CD155 binding with CD226, a T cell stimulatory receptor. LAG-3 is expressed on CD4 and CD8 T cells, and suppresses T cells once it is engaged by one of its ligands including MHC-II, FGL-1, or galectin-3. These two immune checkpoints are now emerging as promising targets in the post-PD-1/CTLA-4 era in cancer immunotherapy. Strong preclinical evidence supports blocking the TIGIT pathway for cancer immunotherapy, although a recent Phase III clinical trial of anti-TIGIT tiragolumab combined with anti-PD-L1 (Tecentriq) failed. Much remains to be learned about the LAG-3 pathway, including the relative importance of its three potential ligands. However, the clinical efficacy of LAG-3 inhibition has been attested, and an anti-LAG-3 antibody (relatlimab) has been approved by the FDA in combination therapy with anti-PD-1 (nivolumab) for the management of patients with unresectable or metastatic melanoma. These recent developments highlight the promise of simultaneously targeting PD-1/PD-L1 and TIGIT or LAG-3 pathways for cancer immunotherapy.

Although combination immune checkpoint therapy is currently the prevailing strategy to overcome resistance to anti-PD-1/PD-L1 therapy, new technology platforms now allow for the development of a different class of therapeutic antibodies, i.e., multispecific antibodies, which can target different epitopes on two or more checkpoint molecules. In addition to blocking multiple inhibitory pathways, such multispecific agents can have additional MOAs, including cell-cell bridging via the crosslinking of antigens on neighboring cells to achieve greater T cell activation and/or cancer cell killing. Another factor to consider when designing therapeutic antibodies is whether to retain Fc function, which plays important roles through ADCC, ADCP, and other mechanisms that contribute to the efficacy of these antibodies. It has been shown that the activities of PD-L1 antibodies rely on FcγR engagement, while those of anti-PD-1 Abs are FcγR-independent. In addition to inducing effector functions such as ADCC, ADCP, and CDC, Fc engagement with FcγR on APCs contribute to stronger T cell activation. The importance of Fc binding for TIGIT antibody efficacy has been a matter of debate, but results from most recent studies are in favor of retaining or enhancing Fc binding.

We have developed multispecific antibodies targeting PD-L1, TIGIT, and LAG-3 and evaluated them with carefully designed in vitro and in vivo assays, which show that these antibodies efficiently promote T cell activation and cancer cell killing and suppress tumor growth. We further show that their superior efficacy compared to antibody combinations depends on the function of the Fc region and the presence of monocytes. As monocytes/macrophages are a major component of tumor-infiltrating immune cells along with T cells in the tumor microenvironment, our antibodies are likely to outperform current benchmark antibodies in cancer immunotherapy.

### PD-L1, TIGIT, and LAG3

Programmed death-ligand 1 (PD-L1) also known as cluster of differentiation 274 (CD274) or B7 homolog 1 (B7-H1) is a protein that in humans is encoded by the CD274 gene. PD-L1 is a type 1 transmembrane protein that has been speculated to play a major role in suppressing the immune system during particular events such as pregnancy, tissue allografts, autoimmune disease and other disease states such as hepatitis. PD-1 (programmed death-1) is mainly expressed on the surfaces of T cells and primary B cells; two ligands of PD-1 (PD-L1 and PD-L2) are widely expressed in antigen-presenting cells (APCs). The interaction of PD-1 with its ligands plays an important role in the negative regulation of the immune response. PD-L1 protein expression can be detected in many human tumor tissues. The microenvironment of tumor site can induce the expression of PD-L1 on tumor cells. PD-L1 expression is beneficial to the occurrence and growth of tumors. It can induce apoptosis of anti-tumor T cells, and thus allow tumor to escape from the immune system attacks. Inhibition the binding between PD-1 and its ligand can make the tumor cells exposed to the killing effect of the immune system, and thus can reach the effect of killing tumor tissues and treating cancers.

TIGIT is a type I transmembrane protein expressed on the surface of T cells and NK cells. It has immunoglobulin domain, transmembrane region and immunoreceptor protein tyrosine inhibitory motif. It is an immunosuppressive co-stimulatory molecule. Immunotherapy is an important area in tumor research. Clinical studies have shown that the treatment targeting the inhibitory receptors of T cells can have significant treatment effect. When receiving the stimulation from an anti-TIGIT agonistic monoclonal antibody, TIGIT, as a receptor, is capable of inhibiting the activity of T cells and NK cells. TIGIT can also act as a ligand functioning on the dendritic cell (DC) surface of the poliovirus receptor (PVR), promote DC secretion of IL-10, and thus inhibiting the immune response. TIGIT is highly expressed in chronic viral infections and in cancers, and is up-regulated in tumor-infiltrating T cells. Therefore, anti-TIGIT antibodies can be used in cancer treatment. While the inhibition of PD-L1 or TIGIT alone may not yield good result, an inhibition of both at the same time can significantly improve CD8-mediated inhibition of tumor proliferation. In addition, TIGIT ligands CD155 and CD112 are overexpressed in some tumor cells, such as colorectal cancer, gastric cancer, neuroblastoma, etc. These ligands can bind to TIGIT, thereby inhibiting the immune response of T cells, leading to tumor cell escape.

LAG-3 (CD223) is a co-inhibitory receptor of T cells. Expression of LAG-3 has been reported in activated CD4+ and CD8+ effector T cells, CD4+Foxp3+ Treg, Tr1 cells, B cells, plasmacytoid DCs, and NK cells. LAG-3 associates with CD3 in the TCR complex and crosslinking of LAG-3 together with CD3 negatively regulates signal transduction leading to reduced T cell proliferation and cytokine production. LAG-3 deficient OVA-specific CD4+ T cells show uncontrolled expansion upon immunization with their cognate antigen. Similarly, increased proliferation of LAG-3 deficient donor T cells causes more severe acute GVHD. On CD8+ T cells, LAG-3 expression is induced by T cell activation and, like in CD4+ T cells, blockade of LAG-3 improves cytotoxic T cell (CTL) proliferation and effector function. In Tregs, loss of LAG-3 reduced the suppressive function of Tregs, while forced expression of LAG-3 conferred effector T cells with suppressive capacity. LAG-3 thus plays an important role in dampening immune responses by functionally contributing to immune suppression by regulatory T cells.

The roles of PD-L1, TIGIT, and LAG3 in cancer are described, e.g., Brahmer et al. “Safety and activity of anti-PD-L1 antibody in patients with advanced cancer.” New England Journal of Medicine 366.26 (2012): 2455-2465; He et al. “CD155T/TIGIT signaling regulates CD8+ T-cell metabolism and promotes tumor progression in human gastric cancer.” Cancer research 77.22 (2017): 6375-6388; and Maruhashi, T. et al. “LAG-3: from molecular functions to clinical applications.” Journal for Immunotherapy of Cancer 8.2 (2020); which are incorporated herein by reference in the entirety.

The present disclosure provides anti-PD-L1 antibodies, anti-TIGIT antibodies, anti-LAG3 antibodies, and multispecific antibodies that target PD-L1, TIGIT, and/or LAG3, and methods of using them for treating various diseases.

### Heavy-Chain Antibody Variable Domain (VHH)

Monoclonal and recombinant antibodies are important tools in medicine and biotechnology. Like all mammals, camelids (e.g., llamas) can produce conventional antibodies made of two heavy chains and two light chains bound together with disulfide bonds in a Y shape (e.g., IgG1). However, they also produce two unique subclasses of IgG: IgG2 and IgG3, also known as heavy chain antibody. These antibodies are made of only two heavy chains, which lack the CH1 region but still bear an antigen-binding domain at their N-terminus called VHH (or nanobody). Conventional Ig require the association of variable regions from both heavy and light chains to allow a high diversity of antigen-antibody interactions. Although isolated heavy and light chains still show this capacity, they exhibit very low affinity when compared to paired heavy and light chains. The unique feature of heavy chain antibody is the capacity of their monomeric antigen binding regions to bind antigens with specificity, affinity and especially diversity that are comparable to conventional antibodies without the need of pairing with another region. This feature is mainly due to a couple of major variations within the amino acid sequence of the variable region of the two heavy chains, which induce deep conformational changes when compared to conventional Ig. Major substitutions in the variable regions prevent the light chains from binding to the heavy chains, but also prevent unbound heavy chains from being recycled by the Immunoglobulin Binding Protein.

The single variable domain of these antibodies (designated VHH, sdAb, nanobody, or heavy-chain antibody variable domain) is the smallest antigen-binding domain generated by adaptive immune systems. The third Complementarity Determining Region (CDR3) of the variable region of these antibodies has often been found to be twice as long as the conventional ones. This results in an increased interaction surface with the antigen as well as an increased diversity of antigen-antibody interactions, which compensates the absence of the light chains. With a long complementarity-determining region 3 (CDR3), VHHs can extend into crevices on proteins that are not accessible to conventional antibodies, including functionally interesting sites such as the active site of an enzyme or the receptor-binding canyon on a virus surface. Moreover, an additional cysteine residue allow the structure to be more stable, thus increasing the strength of the interaction.

VHHs offer numerous other advantages compared to conventional antibodies carrying variable domains (VH and VL) of conventional antibodies, including higher stability, solubility, expression yields, and refolding capacity, as well as better in vivo tissue penetration. Moreover, in contrast to the VH domains of conventional antibodies VHH do not display an intrinsic tendency to bind to light chains. This facilitates the induction of heavy chain antibodies in the presence of a functional light chain loci. Further, since VHH do not bind to VL domains, it is much easier to reformat VHHs into multispecific antibody constructs than constructs containing conventional VH-VL pairs or single domains based on VH domains.

### Anti-LAG3 VHHs

The disclosure provides e.g., anti-LAG3 antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for LAG3-1D1 (or 1D1), and LAG3-1D1 derived antibodies (e.g., humanized antibodies) include CDRs of the VHH domain as set forth in SEQ ID NOs: 1, 2, and 3, respectively. The amino acid sequences for the VHH domain of LAG3-A10 antibody is set forth in SEQ ID NO: 22.

The CDR sequences for LAG3-2G5 (or 2G5), and LAG3-2G5 derived antibodies (e.g., humanized antibodies) include CDRs of the VHH domain as set forth in SEQ ID NOs: 4, 5, and 6, respectively. The amino acid sequence for the VHH domain of LAG3-D7 antibody is set forth in SEQ ID NO: 23.

The CDR sequences for LAG3-3G8-1 (or 3G8-1), and LAG3-3G8-1 derived antibodies (e.g., humanized antibodies) include CDRs of the VHH domain as set forth in SEQ ID NOs: 7, 8, and 9, respectively. The amino acid sequence for the VHH domain of LAG3-3G8-1 antibody is set forth in SEQ ID NO: 24.

The CDR sequences for LAG3-3G8-2 (or 3G8-2), and LAG3-3G8-2 derived antibodies (e.g., humanized antibodies) include CDRs of the VHH domain as set forth in SEQ ID NOs: 10, 11, and 12, respectively. The amino acid sequences for the VHH domain of LAG3-3G8-2 antibody is set forth in SEQ ID NO: 25.

The CDR sequences for LAG3-3G8-3 (or 3G8-3), and LAG3-3G8-3 derived antibodies (e.g., humanized antibodies) include CDRs of the VHH domain as set forth in SEQ ID NOs: 13, 14, and 15, respectively, or SEQ ID NO: 13, SEQ ID NO: 14, and SEQ ID NO: 39 (NGWGDCSGSYRYTPYEYDY), respectively. The amino acid sequences for the VHH domain of LAG3-3G8-3 antibody is set forth in SEQ ID NO: 26

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NOs: 22-26.

Furthermore, in some embodiments, the antibodies or antigen-binding fragments thereof described herein can also contain one, two, or three VHH domain CDRs selected from the group of SEQ ID NOs: 1-3, SEQ ID NOs: 4-6, SEQ ID NOs: 7-9, SEQ ID NOs: 10-12, SEQ ID NOs: 13-15, and SEQ ID NOs: 13, 14, and 39.

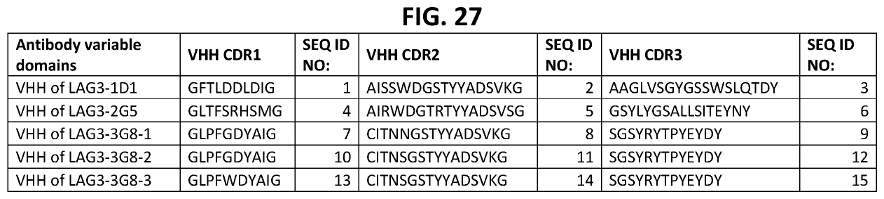

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 27.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 27.

In some embodiments, the antibody or an antigen-binding fragment described herein can contain a heavy-chain antibody variable domain (VHH) containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 39 with zero, one or two amino acid insertions, deletions, or substitutions.

The disclosure also provides antibodies or antigen-binding fragments thereof that bind to LAG3. The antibodies or antigen-binding fragments thereof contain a heavy-chain antibody variable domain (VHH) comprising or consisting of an amino acid sequence that is at least 80%, 85%, 90%, or 95% identical to a selected VHH sequence. In some embodiments, the selected VHH sequence is SEQ ID NO: 22. In some embodiments, the selected VHH sequence is SEQ ID NO: 23. In some embodiments, the selected VHH sequence is SEQ ID NO: 24. In some embodiments, the selected VHH sequence is SEQ ID NO: 25. In some embodiments, the selected VHH sequence is SEQ ID NO: 26.

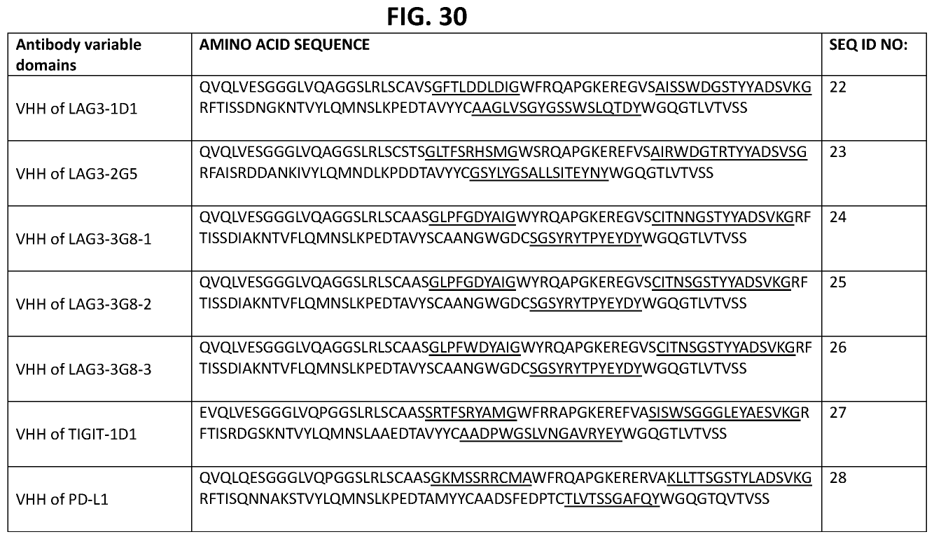

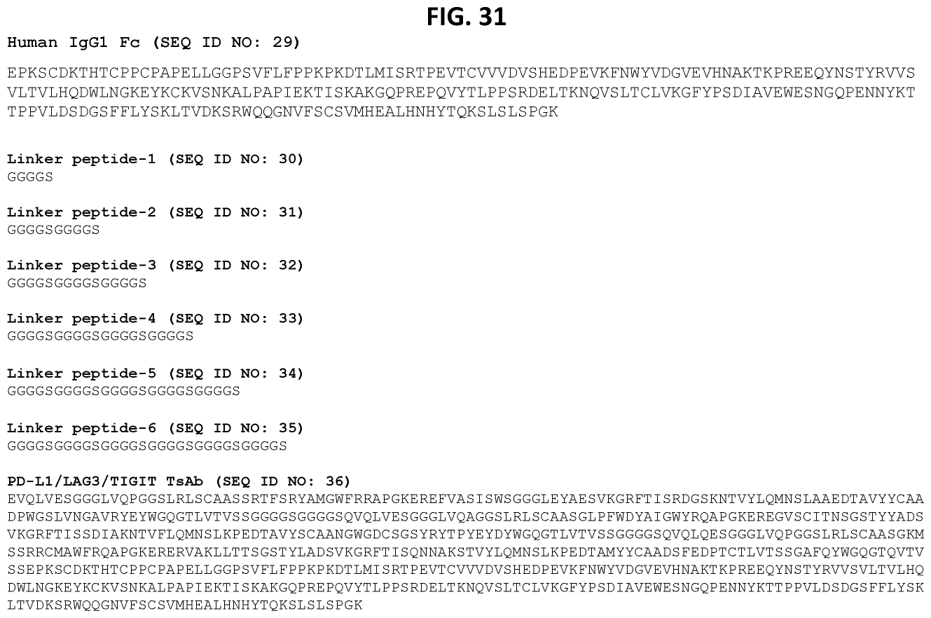

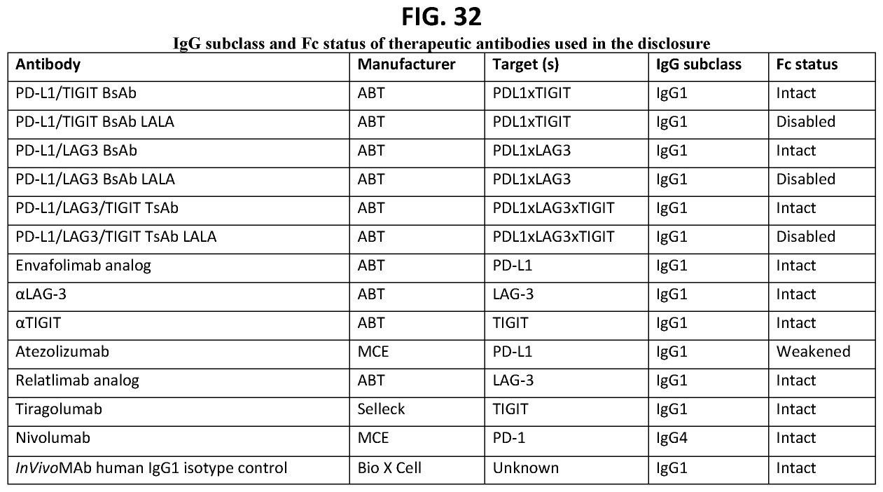

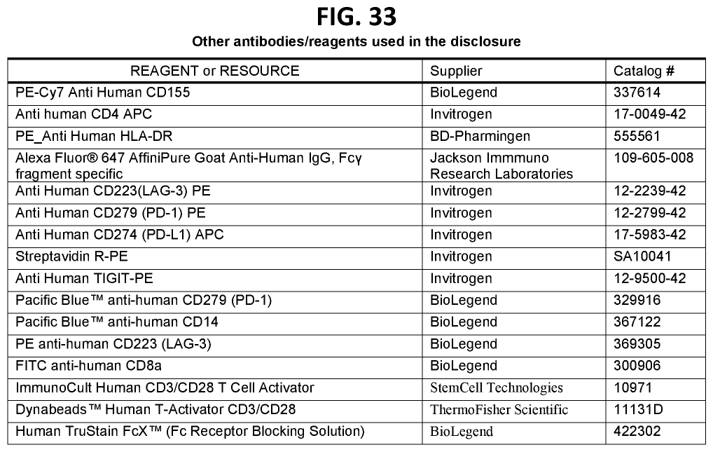

The disclosure also provides nucleic acid comprising a polynucleotide encoding a polypeptide comprising an immunoglobulin heavy-chain antibody variable domain (VHH). The VHH comprises CDRs as shown in FIG. 27, or has sequences as shown in FIG. 30.

The disclosure also provides antibodies or antigen-binding fragments thereof that bind to LAG3. In some embodiments, the antibody or antigen-binding fragment thereof comprises a heavy-chain antibody variable domain (VHH) CDR1 selected from SEQ ID NOs: 1, 4, 7, 10, and 13. In some embodiments, the antibody or antigen-binding fragment thereof comprises a heavy-chain antibody variable domain (VHH) CDR2 selected from SEQ ID NOs: 2, 5, 8, 11, and 14. In some embodiments, the antibody or antigen-binding fragment thereof comprises a heavy-chain antibody variable domain (VHH) CDR3 selected from SEQ ID NOs: 3, 6, 9, 12, 15, and 39.

In one aspect, the disclosure is related to an antibody or antigen-binding fragment thereof comprising the VHH CDRs 1, 2, 3 that are identical to VHH CDRs 1, 2, 3 in SEQ ID NO: 22-26.

### Anti-PD-L1 VHHs

The disclosure provides e.g., anti-PD-L1 antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for PD-L1, and PD-L1 derived antibodies (e.g., humanized antibodies) include CDRs of the VHH domain as set forth in SEQ ID NOs: 19, 20, and 21, respectively, or SEQ ID NO: 19, SEQ ID NO: 20, and SEQ ID NO: 38 (DSFEDPTCTLVTSSGAFQY), respectively. The amino acid sequences for the VHH domain of PD-L1 antibody is set forth in SEQ ID NO: 28.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NO: 28.

Furthermore, in some embodiments, the antibodies or antigen-binding fragments thereof described herein can also contain one, two, or three VHH domain CDRs selected from SEQ ID NOs: 19-21, and SEQ ID NOs: 19, 20, and 38.

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 29.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 29.

In some embodiments, the antibody or an antigen-binding fragment described herein can contain a heavy-chain antibody variable domain (VHH) containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 38 with zero, one or two amino acid insertions, deletions, or substitutions.

The disclosure also provides antibodies or antigen-binding fragments thereof that bind to PD-L1. The antibodies or antigen-binding fragments thereof contain a heavy-chain antibody variable domain (VHH) comprising or consisting of an amino acid sequence that is at least 80%, 85%, 90%, or 95% identical to SEQ ID NO: 28.

The disclosure also provides nucleic acid comprising a polynucleotide encoding a polypeptide comprising an immunoglobulin heavy-chain antibody variable domain (VHH). The VHH comprises CDRs as shown in FIG. 29, or has sequences as shown in FIG. 30.

The disclosure also provides antibodies or antigen-binding fragments thereof that bind to PD-L1. In some embodiments, the antibody or antigen-binding fragment thereof comprises a heavy-chain antibody variable domain (VHH) CDR1 of SEQ ID NO: 19. In some embodiments, the antibody or antigen-binding fragment thereof comprises a heavy-chain antibody variable domain (VHH) CDR2 of SEQ ID NO: 20. In some embodiments, the antibody or antigen-binding fragment thereof comprises a heavy-chain antibody variable domain (VHH) CDR3 of SEQ ID NO: 21 or 39.

In one aspect, the disclosure is related to an antibody or antigen-binding fragment thereof comprising the VHH CDRs 1, 2, 3 that are identical to VHH CDRs 1, 2, 3 in SEQ ID NO: 28.

### Anti-TIGIT VHHs

The disclosure provides e.g., anti-TIGIT antibodies, the modified antibodies thereof, the chimeric antibodies thereof, and the humanized antibodies thereof. The disclosure also provides VHH of these antibodies. These VHHs can be used in various multispecific antibody constructs as described herein.

The CDR sequences for TIGIT, and TIGIT derived antibodies (e.g., humanized antibodies) include CDRs of the VHH domain as set forth in SEQ ID NOs: 16, 17, and 18, respectively, or SEQ ID NO: 16, SEQ ID NO: 17, and SEQ ID NO: 37 (DPWGSLVNGAVRYEY), respectively. The amino acid sequences for the VHH domain of TIGIT antibody is set forth in SEQ ID NO: 27.

The amino acid sequences for various modified or humanized VHH are also provided. As there are different ways to modify or humanize a llama antibody (e.g., a sequence can be modified with different amino acid substitutions), the heavy chain and the light chain of an antibody can have more than one version of humanized sequences. In some embodiments, the humanized VHH domain is at least 80%, 85%, 90%, 91%, 92%, 93%, 94%, 95%, 96%, 97%, 98%, or 99% identical to any sequence of SEQ ID NO: 27.

Furthermore, in some embodiments, the antibodies or antigen-binding fragments thereof described herein can also contain one, two, or three VHH domain CDRs selected from SEQ ID NOs: 16-18, and SEQ ID NOs: 16, 17, and 37.

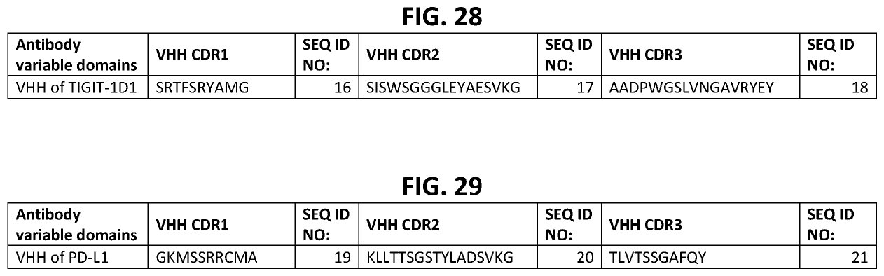

In some embodiments, the antibodies can have a heavy-chain antibody variable domain (VHH) comprising complementarity determining regions (CDRs) 1, 2, 3, wherein the CDR1 region comprises or consists of an amino acid sequence that is at least 80%, 85%, 90%, or 95% identical to a selected VHH CDR1 amino acid sequence, the CDR2 region comprises or consists of an amino acid sequence that is at least 80%, 85%, 90%, or 95% identical to a selected VHH CDR2 amino acid sequence, and the CDR3 region comprises or consists of an amino acid sequence that is at least 80%, 85%, 90%, or 95% identical to a selected VHH CDR3 amino acid sequence. The selected VHH CDRs 1, 2, 3 amino acid sequences is shown in FIG. 28.

In some embodiments, the antibody or an antigen-binding fragment described herein can contain a heavy-chain antibody variable domain (VHH) containing one, two, or three of VHH CDR1 with zero, one or two amino acid insertions, deletions, or substitutions; VHH CDR2 with zero, one or two amino acid insertions, deletions, or substitutions; VHH CDR3 with zero, one or two amino acid insertions, deletions, or substitutions, wherein VHH CDR1, VHH CDR2, and VHH CDR3 are selected from the CDRs in FIG. 28.

In some embodiments, the antibody or an antigen-binding fragment described herein can contain a heavy-chain antibody variable domain (VHH) containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy-chain antibody variable domain (VHH) containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 37 with zero, one or two amino acid insertions, deletions, or substitutions.

The disclosure also provides antibodies or antigen-binding fragments thereof that bind to TIGIT. The antibodies or antigen-binding fragments thereof contain a heavy-chain antibody variable domain (VHH) comprising or consisting of an amino acid sequence that is at least 80%, 85%, 90%, or 95% identical to SEQ ID NO: 27.

The disclosure also provides nucleic acid comprising a polynucleotide encoding a polypeptide comprising an immunoglobulin heavy-chain antibody variable domain (VHH). The VHH comprises CDRs as shown in FIG. 28, or has sequences as shown in FIG. 30.

The disclosure also provides antibodies or antigen-binding fragments thereof that bind to TIGIT. In some embodiments, the antibody or antigen-binding fragment thereof comprises a heavy-chain antibody variable domain (VHH) CDR1 of SEQ ID NO: 16. In some embodiments, the antibody or antigen-binding fragment thereof comprises a heavy-chain antibody variable domain (VHH) CDR2 of SEQ ID NO: 17. In some embodiments, the antibody or antigen-binding fragment thereof comprises a heavy-chain antibody variable domain (VHH) CDR3 of SEQ ID NO: 18 or 37.

In one aspect, the disclosure is related to an antibody or antigen-binding fragment thereof comprising the VHH CDRs 1, 2, 3 that are identical to VHH CDRs 1, 2, 3 in SEQ ID NO: 27.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination numbering scheme (e.g., a combination of Kabat and Chothia).

The antibodies and antigen-binding fragments can also be antibody variants (including derivatives and conjugates) of antibodies or antibody fragments and multi-specific (e.g., bi-specific or tri-specific) antibodies or antibody fragments. Additional antibodies provided herein are polyclonal, monoclonal, multi-specific (multimeric, e.g., bi-specific or tri-specific), human antibodies, chimeric antibodies (e.g., human-mouse chimera), single-chain antibodies, intracellularly-made antibodies (i.e., intrabodies), and antigen-binding fragments thereof.

In some embodiments, the antibodies or antigen-binding fragments thereof comprises an Fc domain that can be originated from various types (e.g., IgG, IgE, IgM, IgD, IgA, and IgY), class (e.g., IgG1, IgG2, IgG3, IgG4, IgA1, and IgA2), or subclass. In some embodiments, the Fc domain is originated from an IgG antibody or antigen-binding fragment thereof. In some embodiments, the Fc domain comprises one, two, three, four, or more heavy chain constant regions.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of illustration, the comparison of sequences and determination of percent identity between two sequences can be accomplished, e.g., using a Blossum 62 scoring matrix with a gap penalty of 12, a gap extend penalty of 4, and a frameshift gap penalty of 5.

### Structures of Multispecific Antibodies

The present disclosure provides multispecific antibodies that bind to TIGIT, LAG3, and/or PD-L1. In some embodiments, the multispecific antibodies are designed to include one or more VHHs that target TIGIT, one or more VHHs that target LAG3, and one or more VHHs that target PD-L1. In some embodiments, the multispecific antibody is a bispecific antibody. In some embodiments, the multispecific antibody is a trispecific antibody. Some of the bispecific or trispecific antibodies with specific structures are described below.

### Structure of BsAb

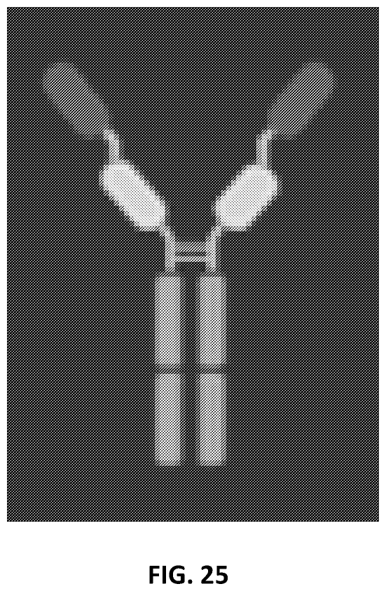

As shown in FIG. 25, a bispecific antibody can be prepared. Specifically, the TIGIT/PD-L1 bispecific antibody comprises (a) a first polypeptide comprising from N-terminus to C-terminus: a first heavy-chain antibody variable domain (VHH1), a first linker peptide sequence, a second heavy-chain antibody variable domain (VHH2), and a first Fc region (optionally including a hinge region); (b) a second polypeptide comprising from N-terminus to C-terminus a second polypeptide comprising from N-terminus to C-terminus: a third heavy-chain antibody variable domain (VHH3), a second linker peptide sequence, a fourth heavy-chain antibody variable domain (VHH4), and a second Fc region (optionally including a hinge region).

In some embodiments, the VHH1 and/or VHH3 specifically bind to TIGIT. In some embodiments, the VHH1 and/or VHH3 specifically bind to LAG3. In some embodiments, the VHH1 and/or VHH3 specifically bind to PD-L1. In some embodiments, the VHH2 and VHH4 specifically bind to TIGIT. In some embodiments, the VHH2 and VHH4 specifically bind to LAG3. In some embodiments, the VHH2 and VHH4 specifically bind to PD-L1.

In some embodiments, the first Fc region and/or the second Fc region comprise a sequence that is at least 80%, 85%, 90%, 95%, or 100% identical to SEQ ID NO: 29. In some embodiments, the TIGIT/PD-L1 bispecific antibody comprises knob-into-hole mutations. In some embodiments, the Fc region is an IgG1 Fc region. In some embodiments, the effector function (e.g., ADCC or CDC) of the Fc region is retained. In some embodiments, the effector function (e.g., ADCC or CDC) of the Fc region is silenced.

In some embodiments, the linker peptide sequence comprises a sequence that is at least 80% identical to any one of SEQ ID NOs: 30-35. In some embodiments, the linker peptide sequence described herein comprises a sequence that is at least 80%, 85%, 90%, 95%, or 100% identical to one or more (e.g., 1, 2, 3, 4, 5, 6, 7, or 8) repeats of GGGGS (SEQ ID NO: 30).

### Structure of TsAb

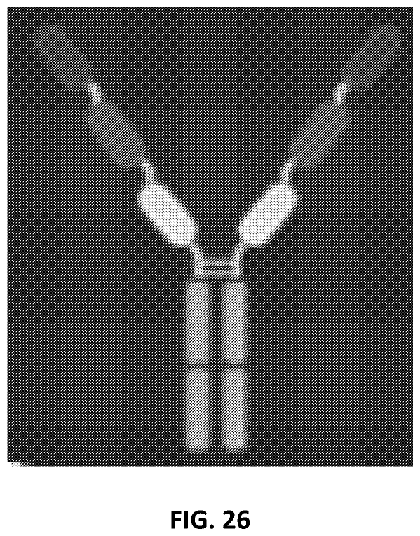

As shown in FIG. 26, a trispecific antibody can be prepared. In some embodiments, the first polypeptide of the BsAb structure described herein can further comprise a fifth heavy-chain antibody variable domain (VHH5). In some embodiments, the VHH5 specifically binds to LAG3. In some embodiments, the VHH5 specifically binds to TIGIT. In some embodiments, the VHH5 specifically binds to PD-L1. In some embodiments, the VHH5 is linked to the N-terminus of the VHH1 via a third linker peptide sequence.

In some embodiments, the second polypeptide of the BsAb structure described herein can further comprise a sixth heavy-chain antibody variable domain (VHH6). In some embodiments, the VHH6 specifically binds to LAG3. In some embodiments, the VHH6 specifically binds to TIGIT. In some embodiments, the VHH6 specifically binds to PD-L1. In some embodiments, the VHH6 is linked to the N-terminus of the VHH2 via a fourth linker peptide sequence.

In some embodiments, the linker peptide sequence comprises a sequence that is at least 80% identical to any one of SEQ ID NOs: 30-35. In some embodiments, the linker peptide sequence described herein comprises a sequence that is at least 80%, 85%, 90%, 95%, or 100% identical to one or more (e.g., 1, 2, 3, 4, 5, 6, 7, or 8) repeats of GGGGS (SEQ ID NO: 30).

In some embodiments, any one of the VHH described herein (e.g., VHH1, VHH2, VHH3, VHH4, VHH5, and/or VHH6) that specifically binds to TIGIT, LAG3, or PD-L1 of the multispecific antibodies described herein can a BsAb or TsAb structures described herein.

In some embodiments, the first polypeptide and/or the second polypeptide of the trispecific antibody (e.g., PD-L1/LAG3/TIGIT TsAb) comprises or consists of an amino acid sequence that is at least 80%, 85%, 90%, 91%, 92%, 93%, 94%, 95%, 96%, 97%, 98%, 99%, or 100% identical to SEQ ID NO: 36.

### Antibody Characteristics

The anti-TIGIT, anti-LAG3, anti-PD-L1, anti-TIGIT/PD-L1, anti-LAG3/PD-L1, or anti-TIGIT/LAG3/PD-L1 antigen-binding protein construct (e.g., antibodies, bispecific antibodies, trispecific antibodies, multi-specific antibodies, or antibody fragments thereof) can include an antigen binding site that is derived from any anti-TIGIT antibody, anti-LAG3, anti-PD-L1 antibody, or any antigen-binding fragment thereof as described herein.

In some embodiments, the antibodies or antigen-binding fragments thereof described herein are PD-1 pathway antagonist. In some embodiments, the antibodies or antigen-binding fragments thereof as described herein are TIGIT antagonist. In some embodiments, the antibodies or antigen-binding fragments thereof are TIGIT agonist. In some embodiments, the antibodies or antigen-binding fragments thereof as described herein are LAG3 antagonist. In some embodiments, the antibodies or antigen-binding fragments thereof are LAG3 agonist.

In some embodiments, the antibodies, or antigen-binding fragments thereof described herein can bind to TIGIT, LAG3, and/or PD-L1, thereby blocking the interaction of these receptors and their respective ligands; increasing immune responses; and/or directly killing the cancer cells by ADCC and/or CDC.

In some embodiments, the antibodies or antigen-binding fragments thereof described herein has a TIGIT binding capability (e.g., determined by ELISA) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody (e.g., Tiragolumab).

In some embodiments, the antibodies or antigen-binding fragments thereof described herein has a PD-L1 binding capability (e.g., determined by ELISA) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody (e.g., Atezolizumab).

In some embodiments, the antibodies or antigen-binding fragments thereof described herein can block the interaction of TIGIT and a TIGIT ligand (e.g., CD155). In some embodiments, the antibodies or antigen-binding fragments thereof decrease binding of TIGIT to a TIGIT ligand (e.g., CD155) to less than 90%, less than 80%, less than 70%, less than 60%, less than 50%, less than 40%, less than 30%, less than 20%, less than 10%, or less than 5% as compared to that of an isotype control antibody.

In some embodiments, the antibodies or antigen-binding fragments thereof can block the interaction between LAG3 and MHC class II molecules. In some embodiments, the antibodies or antigen-binding fragments thereof can block the interaction between LAG3 and FGL1. In some embodiments, the antibodies or antigen-binding fragments thereof can block both the interaction between LAG3 and MHC class II molecules, and the interaction between LAG3 and FGL1.

In some embodiments, the antibodies or antigen-binding fragments thereof described herein can block the interaction of PD-L1 and a PD-1. In some embodiments, the antibodies or antigen-binding fragments thereof decrease binding of PD-L1 to PD-1 to less than 90%, less than 80%, less than 70%, less than 60%, less than 50%, less than 40%, less than 30%, less than 20%, less than 10%, or less than 5% as compared to that of an isotype control antibody. In some embodiments, the antibodies or antigen-binding fragments thereof have a PD-L1/PD-1 blocking capability (e.g., determined by the immunofluorescence assays as described herein).

In some embodiments, the antibody (or antigen-binding fragments thereof) specifically binds to an antigen (e.g., LAG3, PD-L1 or TIGIT) with a dissociation rate (koff) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, or greater than 0.000001 s⁻¹. In some embodiments, kinetic association rates (kon) is greater than 1×10²/Ms, greater than 1×10³/Ms, greater than 1×10⁴/Ms, greater than 1×10⁵/Ms, greater than 2×10⁵/Ms, greater than 2.2×10⁵/Ms or greater than 2.4×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (Kd=koff/kon). In some embodiments, Kd is less than 1×10⁻⁴ M, less than 1×10⁻⁵ M, less than 1×10⁻⁶ M, less than 1×10⁻⁷ M, less than 1×10⁻⁸ M, less than 1×10⁻⁹ M, or less than 1×10⁻¹⁰ M. In some embodiments, the Kd is less than 50 nM, 30 nM, 20 nM, 15 nM, 10 nM, 9 nM, 8 nM, 7 nM, 6 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM. In some embodiments, Kd is greater than 1×10⁻⁴ M, greater than 1×10⁻⁵M, greater than 1×10⁻⁶ M, greater than 1×10⁻⁷M, greater than 1×10⁻⁸ M, greater than 1×10⁻⁹ M, greater than 1×10⁻¹⁰ M, greater than 1×10⁻¹¹ M, or greater than 1×10⁻¹² M. Furthermore, Ka can be deduced from Kd by the formula Ka=1/Kd.

In some embodiments, the binding affinity to TIGIT or PD-L1 is carefully adjusted, e.g., Kd can be between 100 nM-0.1 nM, between 100 nM-1 nM, between 100 nM-10 nM, between 10 nM-0.1 nM, between 10 nM-1 nM, or between 1 nM-0.1 nM.

General techniques for measuring the affinity of an antibody for an antigen include, e.g., ELISA, RIA, and surface plasmon resonance (SPR).

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( T i - T 0 ) / ( Vi - V 0 ) ] \times 100$$

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen binding fragments thereof described herein have the antibody-dependent cell-mediated cytotoxicity (ADCC) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody. In some embodiments, the antibodies or antigen binding fragments thereof described herein can increase antibody-dependent cell-mediated cytotoxicity (ADCC) by at least 10%, 20%, 30%, 40%, 50%, 60%, 70%, 80%, 90%, 100%, 2 folds, 3 folds, 5 folds, 10 folds, 20 folds, or 100 folds, as compared to that of a non-specific antibody control or an isotype antibody control.

In some embodiments, the antibodies or antigen binding fragments thereof described herein have the complement dependent cytotoxicity (CDC) that is at least or about 50%, at least or about 60%, at least or about 70%, at least or about 80%, at least or about 90%, at least or about 100%, at least or about 110%, at least or about 120%, at least or about 130%, at least or about 140%, at least or about 150%, at least or about 200% as compared to that of a reference antibody. In some embodiments, the antibodies or antigen binding fragments thereof described herein can increase complement dependent cytotoxicity (CDC) by at least 10%, 20%, 30%, 40%, 50%, 60%, 70%, 80%, 90%, 100%, 2 folds, 3 folds, 5 folds, 10 folds, 20 folds, or 100 folds, as compared to that of a non-specific antibody control or an isotype antibody control.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the Fc region is human IgG1, human IgG2, human IgG3, or human IgG4.

In some embodiments, the antibodies or antigen binding fragments can induce apoptosis.

In some embodiments, the antibodies or antigen binding fragments do not have a functional Fc region. For example, the antibodies or antigen binding fragments are Fab, Fab′, F(ab′)₂, and Fv fragments.

In some embodiments, the antibodies or antigen binding fragments are humanized antibodies. Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

In some embodiments, the multi-specific antibody including the bispecific antibody described herein (e.g., a TIGIT/PD-L1 or LAG3/PD-L1 bispecific antibody) or the trispecific antibody described herein (e.g., a TIGIT/LAG3/PD-L1 trispecific antibody) has an asymmetric structure comprising: 2, 3, 4, 5, or 6 antigen binding sites. In some embodiments, the multi-specific antibody described herein comprises 2, 3, 4, 5, or 6 antigen binding sites (e.g., antigen binding Fab domains, scFV, or naonbody (VHH)) that target a cancer antigen (e.g., TIGIT, LAG3, or PD-L1). In some embodiments, the C-terminus of a cancer antigen binding Fab domain is connected (e.g., covalently connected or chemically connected) to the N-terminus of a neighboring cancer antigen binding Fab domain within the same multi-specific antibody.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

### Antibodies and Antigen Binding Fragments

The present disclosure provides antibodies and antigen-binding fragments thereof that comprise complementary determining regions (CDRs), heavy chain variable regions, light chain variable regions, heavy chains, or light chains described herein. In some embodiments, the antibodies and antigen-binding fragments thereof are imbalanced bispecific antibodies and antigen-binding fragments thereof.

In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and/or two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); Kontermann, R., & Dübel, S. (Eds.). (2010). Antibody engineering: Volume 2. Springer; each of which is incorporated herein by reference in its entirety. In some embodiments, the CDRs are based on Kabat definition. In some embodiments, the CDRs are based on the Chothia definition. In some embodiments, the CDRs are the longest CDR sequences as determined by Kabat, Chothia, AbM, IMGT, or contact definitions.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, rat, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the scFV has two heavy chain variable domains, and two light chain variable domains. In some embodiments, the scFV has two antigen binding regions (Antigen binding regions: A and B), and the two antigen binding regions can bind to the respective target antigens with different affinities.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the antibodies or antigen-binding fragments thereof can bind to two different antigens or two different epitopes. In some embodiments, the antibodies or antigen-binding fragments thereof can bind to three different antigens or three different epitopes.

An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

In some embodiments, the scFv described herein comprises from N-terminus to C-terminus: VH; the polypeptide linker; and VL. In some embodiments, the scFv described herein comprises from N-terminus to C-terminus: VL; the polypeptide linker; and VH. In some embodiments, the linker peptide comprises a sequence that is at least 80%, 85%, 90%, 95%, or 100% identical to any one SEQ ID NOs: 66-71. In some embodiments, the linker peptide comprises a sequence that is at least or about 80%, 85%, 90%, 95%, or 100% identical to one or more (e.g., 1, 2, 3, 4, 5, 6, 7, or 8) repeats of GGGGS (SEQ ID NO: 66).

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Linear antibodies comprise a pair of tandem Fd segments (VH-CH1-VH-CH1) which, together with complementary light chain polypeptides, form a pair of antigen binding regions. Linear antibodies can be bispecific or monospecific.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

Antibodies and antibody fragments of the present disclosure can be modified in the Fc region to provide desired effector functions or serum half-life. In some embodiments, the Fc region in any one of the antibody or antigen-binding fragment described herein comprises an aspartic acid (Asp) at position 239 according to EU numbering. In some embodiments, the Fc region in any one of the antibody or antigen-binding fragment described herein comprises a glutamic acid (Glu) at position 332 according to EU numbering. In some embodiments, the Fc region described herein is any one of the Fc regions described herein, comprising an aspartic acid (Asp) at position 239 according to EU numbering, and/or a glutamic acid (Glu) at position 332 according to EU numbering. In some embodiments, the Asp239 and/or Glu332 described herein can increase effector functions (e.g., ADCC or CDC) of an antibody or antigen binding fragment thereof by at least or about 10%, 20%, 30%, 40%, 50%, 60%, 70%, 80%, 90%, 1-fold, 2-fold, 5-fold, 10-fold, 20-fold, 50-fold, or 100-fold, as compared to those of a wild-type antibody or antigen-binding fragment thereof. Details can be found, e.g., in Lazar, G. A. et al., “Engineered antibody Fe variants with enhanced effector function.” Proceedings of the National Academy of Sciences 103.11 (2006): 4005-4010, which is incorporated herein by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments (e.g., bispecific antibodies) described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the multi-specific antibody or antigen-binding fragment thereof described herein (e.g., a TIGIT/PD-L1 or LAG3/PD-L1 bispecific antibody or a TIGIT/LAG3/PD-L1 trispecific antibody) binds to an antigen (e.g., TIGIT, LAG3, or PD-L1) with a binding affinity that is about 50%, about 60%, about 70%, about 80%, about 90%, about 100%, about 110%, about 120%, about 130%, about 140%, about 150%, or about 200% to that of a heavy-chain antibody (e.g., an anti-TIGIT, anti-LAG3, or anti-PD-L1 heavy-chain antibody) comprising the same VHH of the multi-specific antibody.

In some embodiments, the bispecific antibody or antigen-binding fragment thereof described herein (e.g., a TIGIT/PD-L1 or LAG3/PD-L1 bispecific antibody or a TIGIT/LAG3/PD-L1 trispecific antibody) mediates ADCC or CDC to at least or about 1 fold, 2 folds, 3 folds, 4 folds, 5 folds, 6 folds, 7 folds, 8 folds, 9 folds, 10 folds, 11 folds, 12 folds, 13 folds, 14 folds, 15 folds, 16 folds, 17 folds, 18 folds, 19 folds, 20 folds, 30 folds, 40 folds, or 50 folds as compared to that mediated by an isotype control antibody.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRITS available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., an antibody or antigen-binding fragment thereof, a multi-specific antibody or antigen-binding fragment thereof, or a polypeptide complex) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

The disclosure also provides a nucleic acid sequence that is at least 1%, 2%, 3%, 4%, 5%, 6%, 7%, 8%, 9%, 10%, 15%, 20%, 25% 30%, 35%0, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10, 1%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein.

The disclosure also provides a nucleic acid sequence that has a homology of at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any nucleotide sequence as described herein, and an amino acid sequence that has a homology of at least 1%, 2%, 3%, 4%, %, 60%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% to any amino acid sequence as described herein.

In some embodiments, the disclosure relates to nucleotide sequences encoding any peptides that are described herein, or any amino acid sequences that are encoded by any nucleotide sequences as described herein. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, 200, 250, 300, 350, or 400 amino acid residues.

In some embodiments, the amino acid sequence (i) comprises an amino acid sequence; or (ii) consists of an amino acid sequence, wherein the amino acid sequence is any one of the sequences as described herein.

In some embodiments, the nucleic acid sequence (i) comprises a nucleic acid sequence; or (ii) consists of a nucleic acid sequence, wherein the nucleic acid sequence is any one of the sequences as described herein.

The percentage of sequence homology (e.g., amino acid sequence homology or nucleic acid homology) can also be determined. How to determine percentage of sequence homology is known in the art. In some embodiments, amino acid residues conserved with similar physicochemical properties (percent homology), e.g. leucine and isoleucine, can be used to measure sequence similarity. Families of amino acid residues having similar physicochemical properties have been defined in the art. These families include e.g., amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). The homology percentage, in many cases, is higher than the identity percentage.

### Methods of Making Antibodies

An isolated fragment of human protein (e.g., TIGIT, LAG3, PD-L1, or cancer antigens) can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of the protein and encompasses an epitope of the protein such that an antibody raised against the peptide forms a specific immune complex with the protein.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide. The preparation can further include an adjuvant, such as Freund's complete or incomplete adjuvant, or a similar immunostimulatory agent.

Polyclonal antibodies can be prepared as described above by immunizing a suitable subject with a polypeptide, or an antigenic peptide thereof (e.g., part of the protein) as an immunogen. The antibody titer in the immunized subject can be monitored over time by standard techniques, such as with an enzyme-linked immunosorbent assay (ELISA) using the immobilized polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

VHH can also be obtained from naïve or designed synthetic llama VHH libraries. PBMC from llamas can be obtained, and RNA can be isolated to generate cDNA by reverse transcription. Then, the VHH genes can be amplified by PCR and cloned to a phage display vector to construct the naïve VHH library. The synthetic (e.g., humanized) VHH library can be prepared by incorporation of shuffled VHH CDRs 1, 2 and 3, generated by overlapping PCR, to a modified human VH scaffold to generate enhanced diversity and keep low immunogenicity. The VHH libraries can be then panned against antigens (e.g., a recombinant human TIGIT protein) to obtain VHH with desired binding affinities.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Phage display (panning) can be used to optimize antibody sequences with desired binding affinities. In this technique, a gene encoding single chain Fv (comprising VH or VL) can be inserted into a phage coat protein gene, causing the phage to “display” the scFv on its outside while containing the gene for the protein on its inside, resulting in a connection between genotype and phenotype. These displaying phages can then be screened against target antigens, in order to detect interaction between the displayed antigen binding sites and the target antigen. Thus, large libraries of proteins can be screened and amplified in a process called in vitro selection, and antibodies sequences with desired binding affinities can be obtained.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Identity or homology with respect to an original sequence is usually the percentage of amino acid residues present within the candidate sequence that are identical with a sequence present within the human, humanized, or chimeric antibody or fragment, after aligning the sequences and introducing gaps, if necessary, to achieve the maximum percent sequence identity, and not considering any conservative substitutions as part of the sequence identity.

In some embodiments, a covalent modification can be made to the antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

In some embodiments, the methods described here are designed to make a bispecific antibody. Bispecific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

In some embodiments, one or more amino acid residues in the CH3 portion of the IgG are substituted. In some embodiments, one heavy chain has one or more of the following substitutions Y349C and T366W. The other heavy chain can have one or more the following substitutions E356C, T366S, L368A, and Y407V. Furthermore, a substitution (-ppcpScp-->-ppcpPcp-) can also be introduced at the hinge regions of both substituted IgG. In some embodiments, one heavy chain has a T366Y (knob) substitution, and the other heavy chain has a Y407T (hole) substitution (EU numbering).

One aspect of the present application provides a heteromultimeric (e.g., heterodimeric) protein comprising a first polypeptide comprising a first heavy chain constant domain 3 (CH3) domain and a second polypeptide comprising a second CH3 domain, wherein the first CH3 domain comprises a substitution relative to a wild-type CH3 domain at amino acid position 354 with a bulky hydrophobic amino acid, and/or the second CH3 domain comprises a substitution relative to a wild-type CH3 domain at amino acid position 347 with a negatively charged amino acid, and wherein the amino acid residue numbering is based on EU numbering. In some embodiments, the bulky hydrophobic amino acid at amino acid position 354 forms a hydrophobic interaction with an amino acid residue in the second CH3 domain. In some embodiments, the second CH3 domain comprises a bulky hydrophobic residue at amino acid position 349 (e.g., Y349). In some embodiments, the negatively charged amino acid at amino acid position 347 forms an ionic bond with an amino acid residue in the first CH3 domain. In some embodiments, the first CH3 domain comprises a positively charged residue at amino acid position 360 (e.g., K360). In some embodiments, the first CH3 domain and the second CH3 domain are human CH3 domains. In some embodiments, the first CH3 domain comprises a substitution selected from the group consisting of S354Y, S354F and S354W. In some embodiments, the first CH3 domain comprises S354Y. In some embodiments, the second CH3 domain does not comprise a compensatory substitution (e.g., a substitution at Y349) for the substitution of S354 in the first CH3 domain. In some embodiments, the second CH3 domain comprises a substitution selected from the group consisting of Q347E and Q³47D. In some embodiments, the second CH3 domain comprises Q347E. In some embodiments according to any one of the heteromultimeric proteins described above, the first CH3 domain and the second CH3 domain further comprise knob-into-hole (KIH) residues. In some embodiments, the knob-into-hole residues are T366Y and Y407T. In some embodiments, the first CH3 domain comprises T366Y and S354Y, and the second CH3 domain comprises Y407T and Q347E. In some embodiments, the first CH3 domain comprises Y407T and S354Y, and the second CH3 domain comprises T366Y and Q347E. Details can be found, e.g., in PCT/US2020/025469, which is incorporated herein by reference.

Furthermore, an anion-exchange chromatography can be used to purify bispecific antibodies. Anion-exchange chromatography is a process that separates substances based on their charges using an ion-exchange resin containing positively charged groups, such as diethyl-aminoethyl groups (DEAE). In solution, the resin is coated with positively charged counter-ions (cations). Anion exchange resins will bind to negatively charged molecules, displacing the counter-ion. Anion exchange chromatography can be used to purify proteins based on their isoelectric point (pI). The isoelectric point is defined as the pH at which a protein has no net charge. When the pH>pI, a protein has a net negative charge and when the pH<pI, a protein has a net positive charge. Thus, in some embodiments, different amino acid substitution can be introduced into two heavy chains, so that the pI for the homodimer comprising two Arm A and the pI for the homodimer comprising two Arm B is different. The pI for the bispecific antibody having Arm A and Arm B will be somewhere between the two pIs of the homodimers. Thus, the two homodimers and the bispecific antibody can be released at different pH conditions. The present disclosure shows that a few amino acid residue substitutions can be introduced to the heavy chains to adjust pI.

Thus, in some embodiments, the amino acid residue at Kabat numbering position 83 is lysine, arginine, or histidine. In some embodiments, the amino acid residues at one or more of the positions 1, 6, 43, 81, and 105 (Kabat numbering) is aspartic acid or glutamic acid.

In some embodiments, the amino acid residues at one or more of the positions 13 and 105 (Kabat numbering) is aspartic acid or glutamic acid. In some embodiments, the amino acid residues at one or more of the positions 13 and 42 (Kabat numbering) is lysine, arginine, histidine, or glycine.

Bispecific antibodies can also include e.g., cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bispecific antibodies from antibody fragments are also known in the art. For example, bispecific antibodies can be prepared using chemical linkage. Brennan et al. (Science 229:81, 1985) describes a procedure where intact antibodies are proteolytically cleaved to generate F(ab′)2 fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bispecific antibody.

### Methods of Treatment

The methods described herein include methods for the treatment of disorders associated with cancer. Generally, the methods include administering a therapeutically effective amount of engineered bispecific antibodies (e.g., imbalanced bispecific antibodies) of antigen-binding fragments thereof as described herein, to a subject who is in need of, or who has been determined to be in need of, such treatment.

As used in this context, to “treat” means to ameliorate at least one symptom of the disorder associated with cancer. Often, cancer results in death; thus, a treatment can result in an increased life expectancy (e.g., by at least 1, 2, 3, 4, 5, 6, 7, 8, 9, 10, 11, 12 months, or by at least 1, 2, 3, 4, 5, 6, 7, 8, 9, 10 years). Administration of a therapeutically effective amount of an agent described herein (e.g., imbalanced bispecific antibodies) for the treatment of a condition associated with cancer will result in decreased number of cancer cells and/or alleviated symptoms.

As used herein, the term “cancer” refers to cells having the capacity for autonomous growth, i.e., an abnormal state or condition characterized by rapidly proliferating cell growth. The term is meant to include all types of cancerous growths or oncogenic processes, metastatic tissues or malignantly transformed cells, tissues, or organs, irrespective of histopathologic type or stage of invasiveness. The term “tumor” as used herein refers to cancerous cells, e.g., a mass of cancerous cells. Cancers that can be treated or diagnosed using the methods described herein include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the agents described herein are designed for treating or diagnosing a carcinoma in a subject. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the cancer is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

In one aspect, the disclosure also provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof, or an antibody drug conjugate disclosed herein to a subject in need thereof, e.g., a subject having, or identified or diagnosed as having, a cancer, e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer cells express a TIGIT ligand (e.g., CD155) and/or PD-L1. In some embodiments, the cancer is a cancer expressing both a LAG3 ligand (e.g., MHC class II molecules and/or FGL1) and PD-L1.

In some embodiments, the disclosure features methods that include: identifying a subject having cancer; and administering a therapeutically effective amount of an antibody or antigen-binding fragment thereof, or an antibody drug conjugate disclosed herein to the subject in need thereof, e.g., a subject having, or identified or diagnosed as having, a cancer.

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the cancer is a cancer expressing TIGIT. In some embodiments, the cancer is a cancer expressing PD-L1. In some embodiments, the cancer is a cancer expressing both a TIGIT ligand (e.g., CD155) and PD-L1. In some embodiments, the cancer is a cancer expressing both a LAG3 ligand (e.g., MHC class II molecules and/or FGL1) and PD-L1.

In some embodiments, the cancer is liver cancer, melanoma, non-small cell lung cancer, head and neck squamous cell carcinoma, relapsed or refractory classical Hodgkin lymphoma, squamous cell lung cancer, renal cell carcinoma, pancreatic cancer, breast cancer, lung cancer, or cutaneous squamous cell carcinoma.

In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is hereditary papillary renal cell carcinomas. In some embodiments, the cancer is gastric, head and neck, liver, ovarian, NSCLC or thyroid cancers.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-drug conjugates, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody, an antigen binding fragment, or an antibody-drug conjugate is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody, antigen binding fragment, or antibody-drug conjugate may vary, depending on, inter alia, patient history as well as other factors such as the type (and/or dosage) of antibody used.

Effective amounts and schedules for administering the antibodies, antibody-encoding polynucleotides, antibody-drug conjugates, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, antibody-drug conjugates, and/or compositions disclosed herein, the route of administration, the particular type of antibodies, antibody-encoding polynucleotides, antigen binding fragments, antibody-drug conjugates, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, antibody-drug conjugates, or pharmaceutical composition (e.g., any of the antibodies, antigen-binding fragments, antibody-drug conjugates, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody, antigen-binding fragment, antibody-drug conjugates, and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, antibody-drug conjugate, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, antibody-drug conjugate, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, antibody-drug conjugate, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments, antibody-drug conjugates (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL 1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody.

In some embodiments, the multi-specific antibody or antigen-binding fragment thereof, and/or the polypeptide complex described herein can induce clustering of an effector cell (e.g., a T cell or NK cell expressing TIGIT and/or PD-1) and a target cell (e.g., an APC cell expressing CD155 and/or PD-L1). In some embodiments, the clustering occurs at the interface of the effector cell and the target cell.

### Pharmaceutical Compositions and Routes of Administration

Also provided herein are pharmaceutical compositions that contain at least one (e.g., one, two, three, or four) of the antibodies, antigen-binding fragments, or antibody-drug conjugates described herein. Two or more (e.g., two, three, or four) of any of the antibodies, antigen-binding fragments, or antibody-drug conjugates described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies, antigen-binding fragments, antibody-drug conjugates described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments, or antibody-drug conjugates described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof, or antibody-drug conjugates for various uses as described herein.

### EXAMPLES

The invention is further described in the following examples, which do not limit the scope of the invention described in the claims.

### Methods and Materials

### Antibodies and Cell Lines

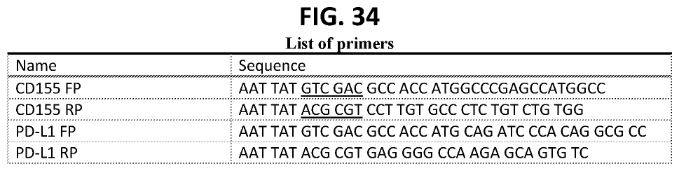

Therapeutic and detection antibodies used in this study are listed in FIGS. 34-35, respectively. All cell lines were obtained from ATCC and maintained at 37° C., 5% CO₂, in RPMI-1640 supplemented with 10% fetal bovine serum (FBS) and 50 mM b-mercaptoethanol.

### Antibody Construction, Expression, and Purification

All the antibodies are VHH type antibodies. Coding regions for the antibodies were synthesized and cloned into expression vector pCDNA3.1 to generate the expression constructs, and their sequences were confirmed by sequencing. For antibody expression and purification, the Expi293 cells were transiently transfected with above plasmids according to manufacturer's instructions. Enhancers were added to cells 17 h after transfection. At 72 h after transfection, the cell culture was centrifuged at 3000 g for 10 min. The supernatant was filtered with a 0.45 μm membrane and the antibody concentration was determined using Protein A probe on Gator (Probe Life). The VHH-Fc or the IgG antibodies were purified using Protein A columns on the AKTA Explorer 100 purification system (buffer A: PBS, pH=7.4; buffer B: 0.1 M Glycine, pH=2.5), and dialyzed in PBS twice. The antibodies were then filtered with a 0.22 μM membrane and used for experiments.

### Generation of Stable Cell Lines

Transgenes were targeted to the AAVS1 safe harbor using the AAVS1 Transgene Knockin kit (Origene) with CRISPR-Cas9 knock-in technology for stable expression. Briefly, the coding region was amplified from template with primers (FIG. 34) by PCR using Q5 Hot Start High-Fidelity DNA Polymerase (NEB) and cloned into the donor vector pAAVS1-EFla-Puro-DNR between the SalI and the MluI sites. The resultant donor construct was co-transfected with the pCas-Guide-AAVS1 vector (coding for Cas9 and AAVS1 guide RNA) into target cells by electroporation using the Neon system (Life Technologies). Puromycin selection for stable cell lines began one week after transfection and continued until untransfected control cells died off Stable expression of the transgene was confirmed by flow cytometry with specific antibodies.

### Cell-Based Antibody Binding Assay

10⁵ cells (RKO, Jurkat TIGIT, and Jurkat LAG-3 for testing antibody binding to PD-L1, TIGIT, and LAG-3, respectively) per well were distributed in 0.1 ml medium to 96-well V-bottom plates. Supernatants were discarded after spinning down cells at 300 g, 5 min at room temperature. Cells were resuspended in 50 ml of a serial dilution of antibodies and incubated for 1 h at room temperature. After incubation, cells were washed by adding 0.15 ml cell staining buffer (PBS containing 1% FBS and 0.1% NaN3) and centrifugation at 300 g, 5 min at room temperature. Supernatants were discarded and cells were washed one more time with 0.15 ml of staining buffer. Cells were subsequently resuspended in 50 ml of secondary antibody (Alexa Fluor 647-conjugated AffiniPure Goat Anti-Human IgG, Fcγ Fragment Specific. Used at 1:500) and incubated at RT for 30 min. Cells were spun down after 0.15 ml cell staining buffer was added, resuspended in 0.12 ml of cell staining buffer, and analyzed by flow cytometry on a NovoCyte flow cytometer (Agilent).

### Cell-Based Antibody Blocking Assay

10⁵ cells (RKO for PD-1/PD-L1 blocking, Raji for MHC-II/LAG-3 blocking, and Jurkat TIGIT for CD155/TIGIT blocking) were distributed per well in 0.1 ml medium to 96-well V-bottom plate in duplicate. Supernatants were discarded after spinning down cells at 300 g, 5 min at room temperature. Cells were resuspended in 50 ml of a serial dilution of antibodies containing 1 mg/ml of biotinylated probes (PD-1, LAG-3, or CD155) and incubated for 1 h at room temperature. After incubation, cells were washed by adding 0.15 ml cell staining buffer (PBS containing 1% FBS and 0.1% NaN3) and centrifugation at 300 g, 5 min at room temperature. Supernatants were discarded and cells were washed one more time with 0.15 ml of staining buffer. Cells were subsequently resuspended in 50 ml of 1:500 Streptavidin-PE and incubated at RT for 30 min. Cells were spun down after 0.15 ml cell staining buffer was added, resuspended in 0.12 ml of cell staining buffer, and analyzed by flow cytometry on a NovoCyte flow cytometer (Agilent).

### Antibody-Mediated Cell-Cell Conjugation

Cancer cells (RKO cells that expresses endogenous PD-L1) and T cells (Jurkat cells transfected with LAG-3 or TIGIT) were labeled with ViaFluor 405 (Bitotium) and ViaFluor 488 (Biotium), respectively, per the manufacturer's instructions. Labeled cancer cells and T cells (5×10⁴ cells each per well) were combined, spun down, and resuspended in 50 ul of a serial dilution of antibodies and incubated for 1 h at room temperature. Cell staining buffer (0.1 ml) was added and samples were analyzed by flow cytometry to quantify VF405/488 double positive events that represent cancer cell-T cell conjugates.

### Monocyte Depletion of PBMCs by Adherence

Human PBMCs in 6-well tissue culture plate were incubated in 37° C. CO₂ incubator to allow monocyte adhesion. Unattached cells were harvested and monocyte frequency in PBMCs with or without depletion was determined by flow cytometry after staining with CD14 antibodies.

### Cell-Based Model of Antitumor Immune Response for Therapeutic Antibody Evaluation

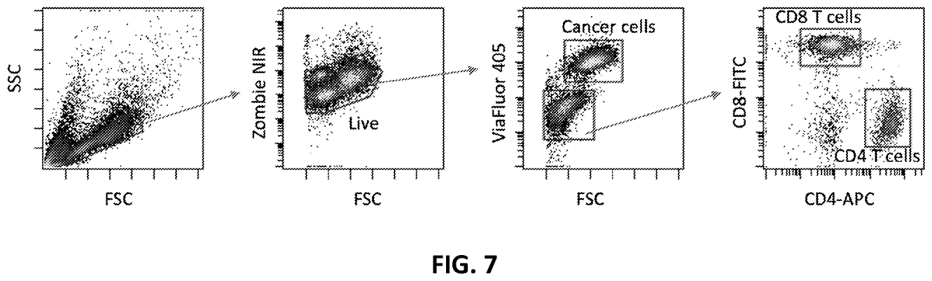

Cancer cells transfected with a transmembrane form of anti-human CD3 antibody (OKT3) scFv were labeled with ViaFluor 405 (Biotium), as described above. Human PBMCs were washed in warmed medium (RMPI-1640 containing 10% FBS and 50 mM b-mercaptoethanol) and combined with labeled cancer cells (5×10⁴ PBMCs and 1×10⁴ cancer cells in 0.15 ml medium for each well). The cell mixture was then transferred to 96-well U-bottom plates containing 50 ul antibody and incubated in a 37° C. CO₂ incubator for 4 days. Cells were then harvested and stained with the Zombie NIR Fixable Viability Kit (BioLegend) per the manufacturer's instructions. The cells were subsequently stained with FITC-labeled anti-human CD8 and APC-labeled anti-human CD4 antibodies (BioLegend) and analyzed by flow cytometry to quantify viable cancer cells (Zombie-VF405+), CD8 T cells (Zombie-CD8+) and CD4 T cells (Zombie-CD4+) (see FIG. 7 for gating strategy).

### Mouse Tumor Model and Treatments

Animal work was outsourced to Biocytogen. NSG female mice at 6-8 weeks of age were subcutaneously injected with 5×10⁶ RKO OKT3 cells and 2.5×10⁶ human PBMCs in Matrigel (1:1) on the right flank. Two weeks later, when tumor size reached a volume of approximately 100 mm³ (day 0), mice were randomized into treatment groups and injected i.v. with 10⁶ human PBMCs and i.p. with 5 mg/kg body weight of PD-L1/TIGIT BsAb, PD-L1/LAG3/TIGIT TsAb, anti-PD-L1 (atezolizumab) plus anti-TIGIT (tiragolumab), or PBS, respectively. Body weight and tumor volume were measured twice weekly starting on day 0. Tumors were measured with a digital caliper and tumor size was calculated using the formula tumor volume (mm³)=L×W²/2.

### Statistical Analysis

Unless noted otherwise, data visualization and statistical analyses were performed using Prism 9.4.0 (GraphPad). P values<0.05 were considered significant. Statistical tests are specified in figure legends.

### Example 1. Generation of Multispecific Antibodies PD-L1/TIGIT BsAb, PD-L1/LAG3 BsAb, and PD-L1/LAG3/TIGIT TsAb and their Target Binding/Blocking Characteristics

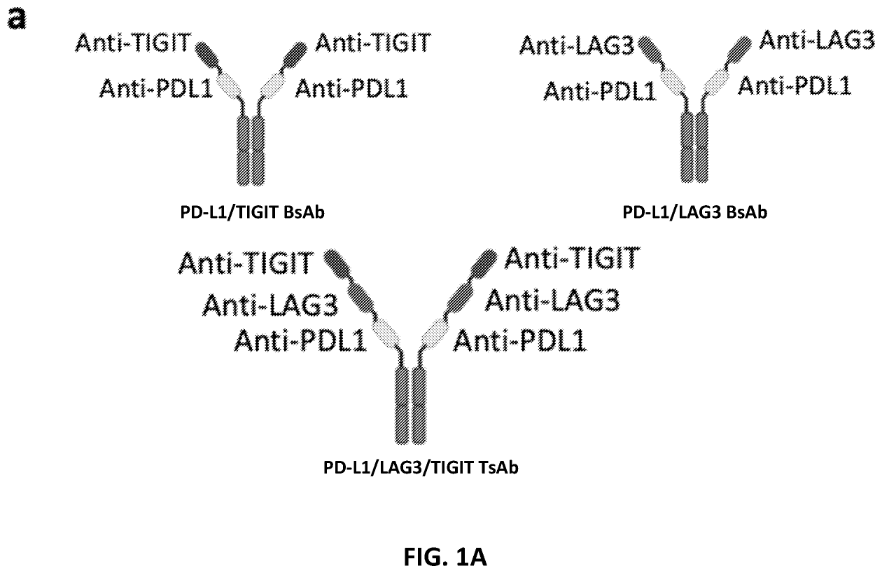

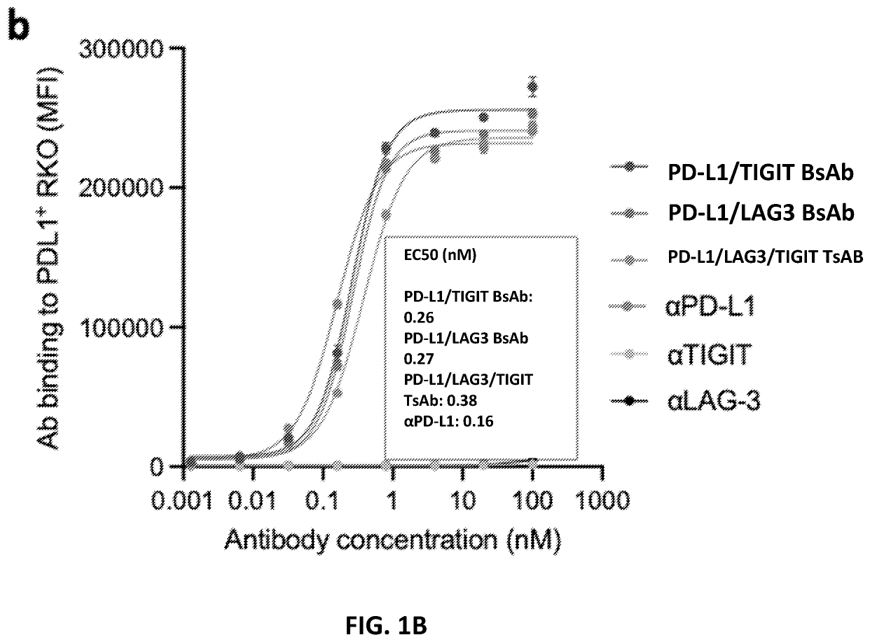

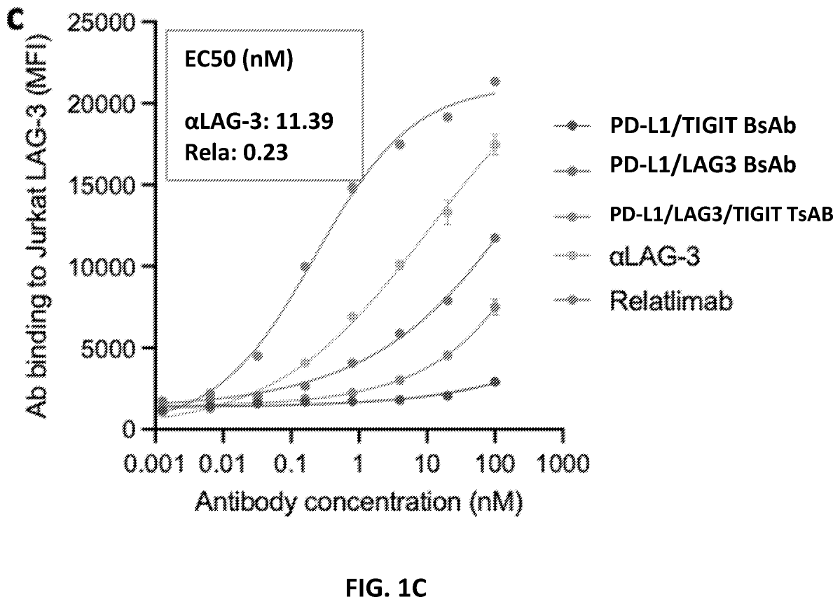

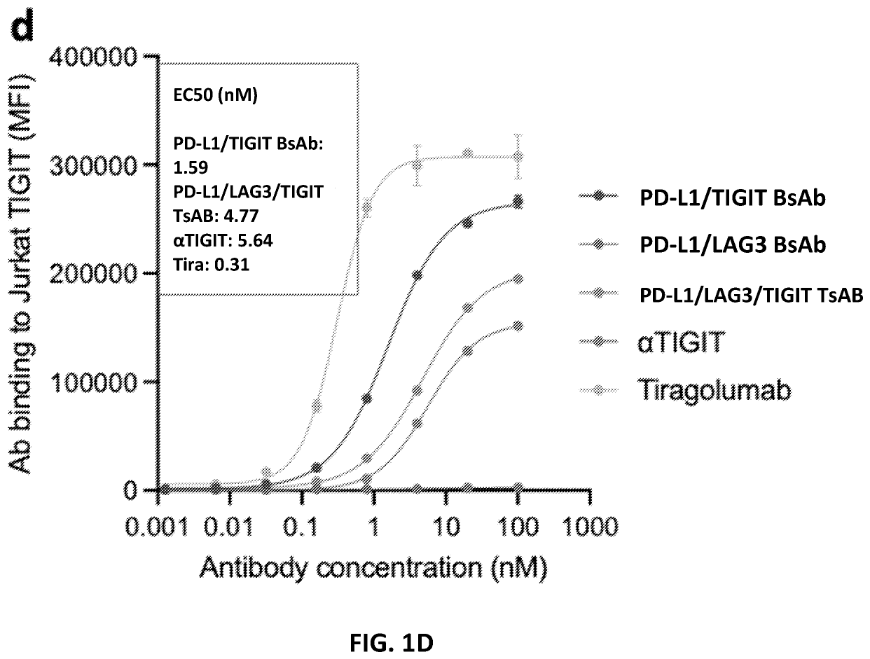

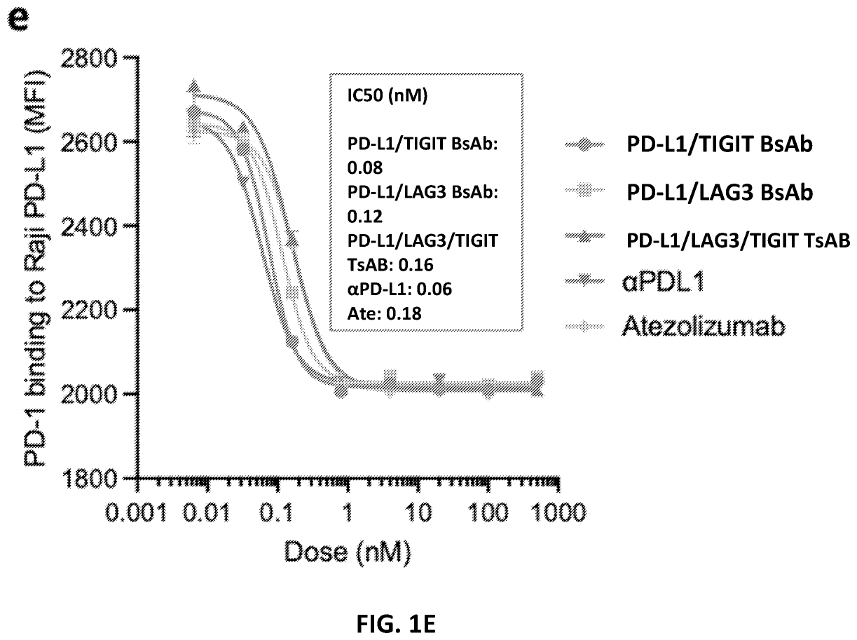

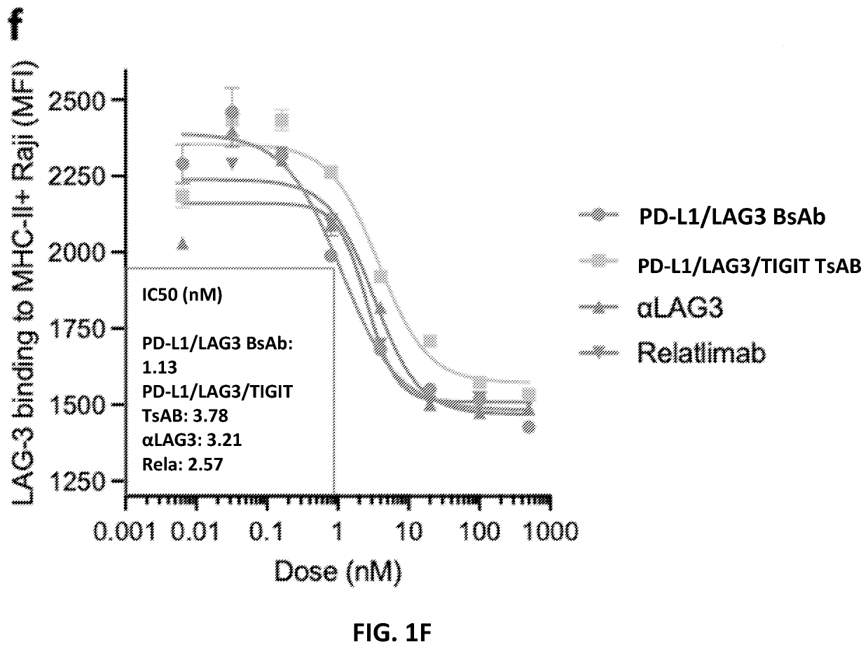

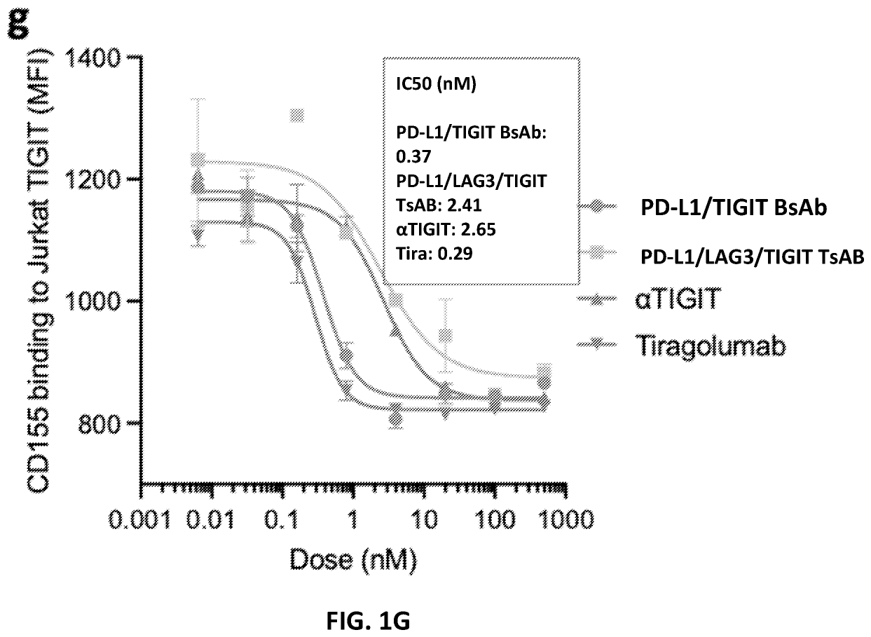

We discovered llama single domain antibodies (VHHs) that bind to TIGIT or LAG-3 by screening our in-house naïve and synthetic humanized llama VHH phage libraries with recombinant TIGIT or LAG-3 protein. The lead binders were further assessed for their ability to block CD155/TIGIT and MHC-II/LAG-3 interactions, respectively. VHHs with high binding affinity and blocking ability were selected and fused into a single PDL1×TIGIT BsAb, PDL1×LAG3 BsAb or a PDL1×LAG3×TIGIT TsAb, with the aid of our computer-aided antibody design (CAAD) platform for optimization (FIG. 1A). The sequence of the PD-L1 binding domain of PD-L1/TIGIT BsAb, PD-L1/LAG3 BsAb, and PD-L1/LAG3/TIGIT TsAb originated from the Alphamab anti-PD-L1 antibody envafolimab. T cells that express LAG-3 or TIGIT were generated by transfection of Jurkat cells with either protein. Binding characteristics of the three antibodies to cell-associated PD-L1, TIGIT, and LAG-3 are shown in FIGS. 1B-1D. Using RKO expressing endogenous PD-L1, we found that PD-L1/TIGIT BsAb, PD-L1/LAG3 BsAb, and PD-L1/LAG3/TIGIT TsAb largely retain the high PD-L1 binding activity of the parental antibody (FIG. 1B). With TIGIT-transfected Jurkat cells, we found that PD-L1/LAG3/TIGIT TsAb has a binding affinity for TIGIT similar to that of ABT's parental anti-TIGIT antibody, while that of PD-L1/TIGIT BsAb is notably higher, and all of them exhibited significantly lower affinity than Roche's tiragolumab (FIG. 1C). Improved binding of PD-L1/TIGIT BsAb to TIGIT compared to the parental TIGIT antibody suggests that the PD-L1-binding domain in PD-L1/TIGIT BsAb exerts a positive effect on TIGIT binding. The LAG-3-binding activity of PD-L1/LAG3/TIGIT TsAb is lower than that of PD-L1/LAG3 BsAb, and both are markedly lower than that of ABT's parental LAG-3 antibody, as well as BMS's relatlimab (FIGS. 1B-1D).

Overall, these results show that PD-L1/TIGIT BsAb, PD-L1/LAG3 BsAb, and PD-L1/LAG3/TIGIT TsAb antibodies have high affinities for PD-L1, similar to that of the monospecific parental antibody envafolimab, and relatively low affinities for TIGIT and LAG-3 compared to that of tiragolumab and relatlimab, respectively. As strong antibody binding to LAG-3 and TIGIT can lead to Fc-mediated ADCC/ADCP of T cells, such “imbalanced” binding characteristics (high affinities for PD-L1 and moderate to low affinities for LAG-3 and TIGIT) can be desirable for efficacy/safety balance.

Consistent with their high PD-L1 binding affinities (FIG. 1B), PD-L1/TIGIT BsAb, PD-L1/LAG3 BsAb, and PD-L1/LAG3/TIGIT TsAb efficiently blocked PD-1/PD-L1 binding, compared with the parental AlphaMab anti-PD-L1 envafolimab, with IC50's around 0.1 nM (FIG. 1E). Interestingly, despite their low LAG-3 binding affinities (FIG. 1c), PD-L1/LAG3 BsAb, and PD-L1/LAG3/TIGIT TsAb blocked LAG3/MHC-II binding with IC50's similar to those of the parental LAG3 antibody and the BMS LAG-3 antibody relatilimab (FIG. 1F). Consistent with the binding data, PD-L1/TIGIT BsAb and the Roche TIGIT antibody tiragolumab blocked CD155/TIGIT binding more efficiently (IC50's ˜0.3 nM) than PD-L1/LAG3/TIGIT TsAb and the parental TIGIT antibody (IC50's ˜2.5 nM) (FIG. 1G).

### Example 2. MsAbs PD-L1/TIGIT BsAb, PD-L1/LAG3 BsAb, and PD-L1/LAG3/TIGIT TsAb Promote Interactions Between T Cells and Cancer Cells

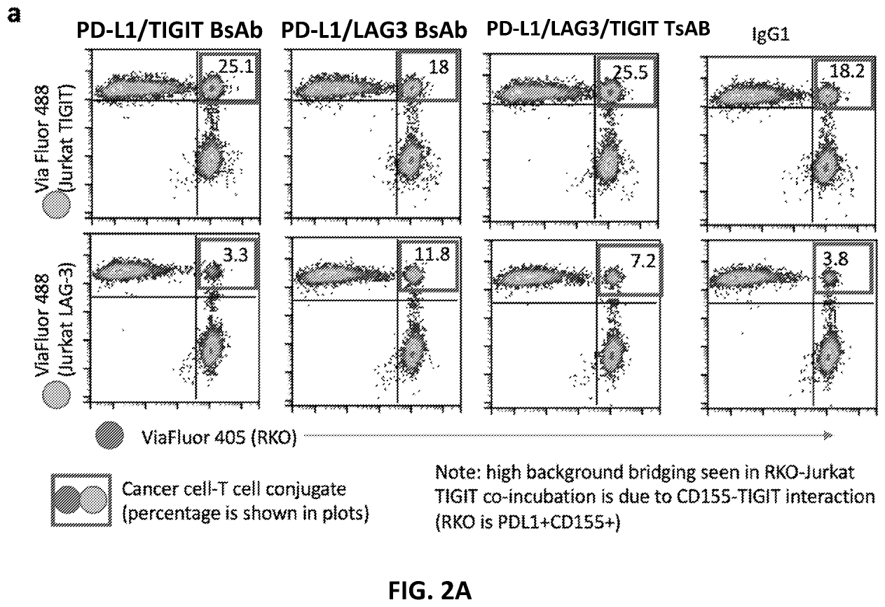

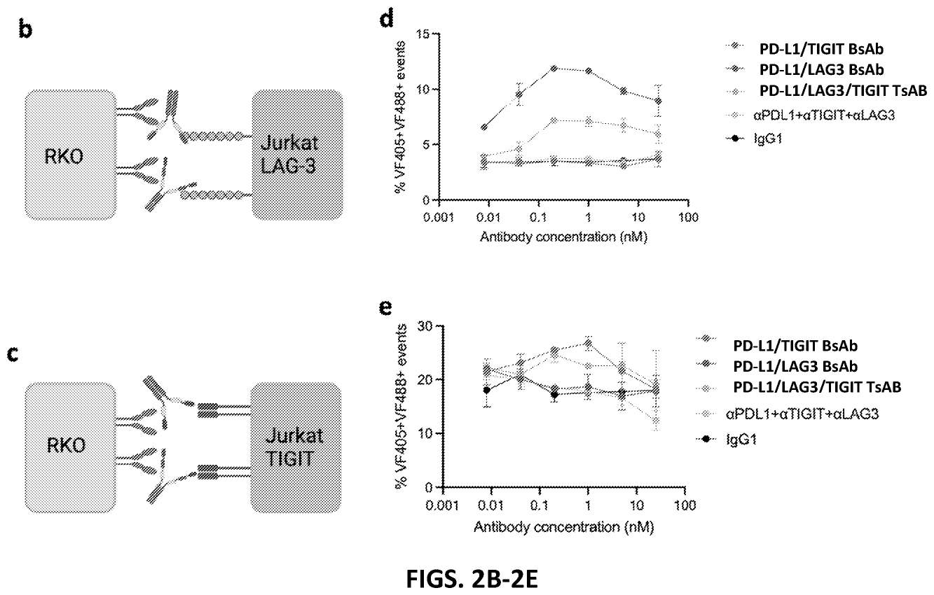

One potential MOA of multispecific antibodies is to promote cell-cell interactions by bridging antigens expressed on respective cells. To test this possibility, we incubated cancer cells (RKO that express endogenous PD-L1) with Jurkat T cells that express LAG-3 (FIGS. 2A-2B) or TIGIT (FIGS. 2C-2D), in the presence of PD-L1/TIGIT BsAb, PD-L1/LAG3 BsAb, PD-L1/LAG3/TIGIT TsAb, or control monospecific antibodies. The cancer cells and the Jurkat cells were pre-labeled with different fluorescent dyes, and cell conjugates were identified as events that are positive for both fluorescent dyes using flow cytometry (FIGS. 2A-2C). Just as there is an optimal antibody/antigen ratio for the formation of polyclonal antibody-antigen complexes, we observed dose-dependent effects of the three multispecific antibodies on cancer cell-T cell conjugation, with maximal bridging between 0.2 and 1 nM, as compared to a combination of monospecific antibodies (FIGS. 2D-2E). At low concentrations, we observed that cell-cell bridging increased with antibody concentration until an optimal concentration is reached. Further increases in antibody concentration beyond this point lead to decreased cell conjugation, due to intense competition for target binding between crosslinked antibodies and excess free antibody molecules. These results demonstrate the dose-dependent ability of our three multispecific antibodies to potentiate interactions of cancer cells and T cells that express different target molecules.

### Example 3. Multispecific Antibodies PD-L1/TIGIT BsAb, PD-L1/LAG3 BsAb, and PD-L1/LAG3/TIGIT TsAb with Wildtype IgG1 Fc Promoted Robust Antitumor T Cell Response In Vitro

PD-L1/TIGIT BsAb, PD-L1/LAG3 BsAb, and PD-L1/LAG3/TIGIT TsAb were designed to target major immune checkpoint molecules that are expressed by cancer cells, antigen-presenting cells (APCs), and T cells in anti-cancer immune responses. In vivo, an antitumor T cell response begins with the activation of T cells by tumor antigens that are presented to the T cell receptor (TCR) by APCs as peptide-MHC complexes. In the TME, activated T cells kill tumor cells but eventually become dysfunctional (exhausted) under the persistent stimulation of tumor antigens. To develop an in vitro assay that best predicts in vivo efficacy of the therapeutics antibodies, we mimicked multiple cell type interactions and T cell exhaustion in the TME that are amenable to antibody treatment. TCR-mediated immune synapse formation between the APC/target cell and T cell is required for efficient T cell activation, immune checkpoint function, and T cell cytotoxicity toward the target cell. We therefore first established a T cell-engaging cancer cell line by transfecting the human colon cancer cell line RKO, which expresses endogenous PD-L1 (a PD-1 ligand) and CD155 (a TIGIT ligand) but not MHC-II (a LAG-3 ligand), with a transmembrane form of anti-human CD3 antibody (OKT3) scFv to engage and activate the TCR. Tumor cells engineered with plasma membrane-expressed anti-CD3 mAb fragments have been used in similar assays.

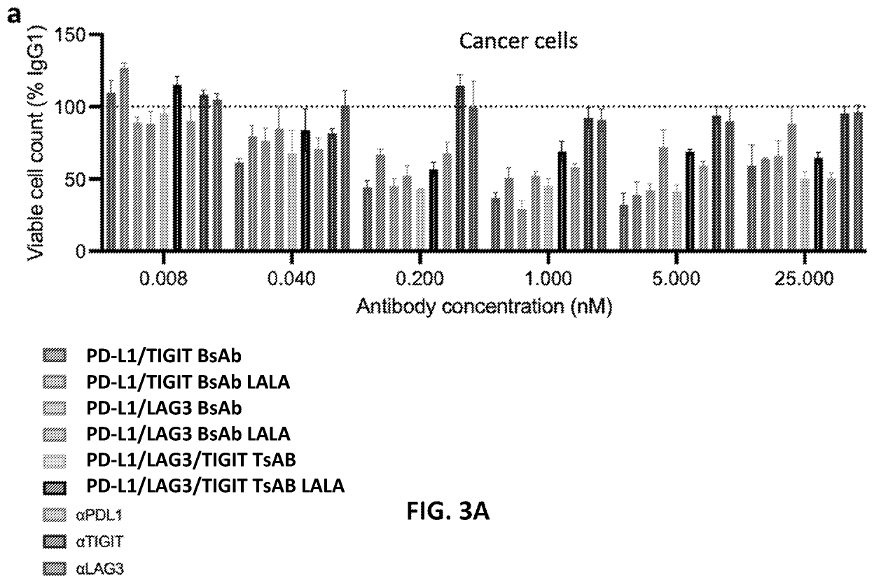

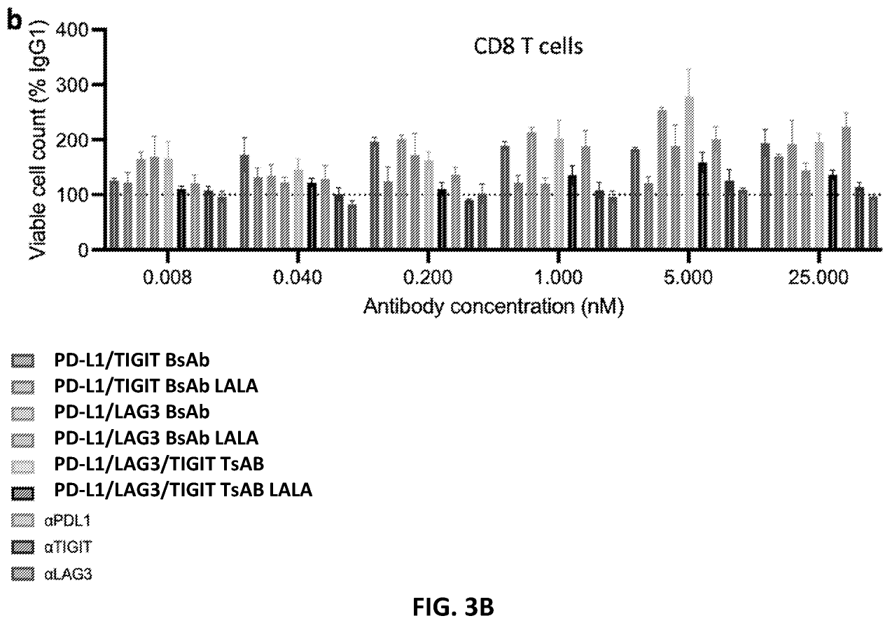

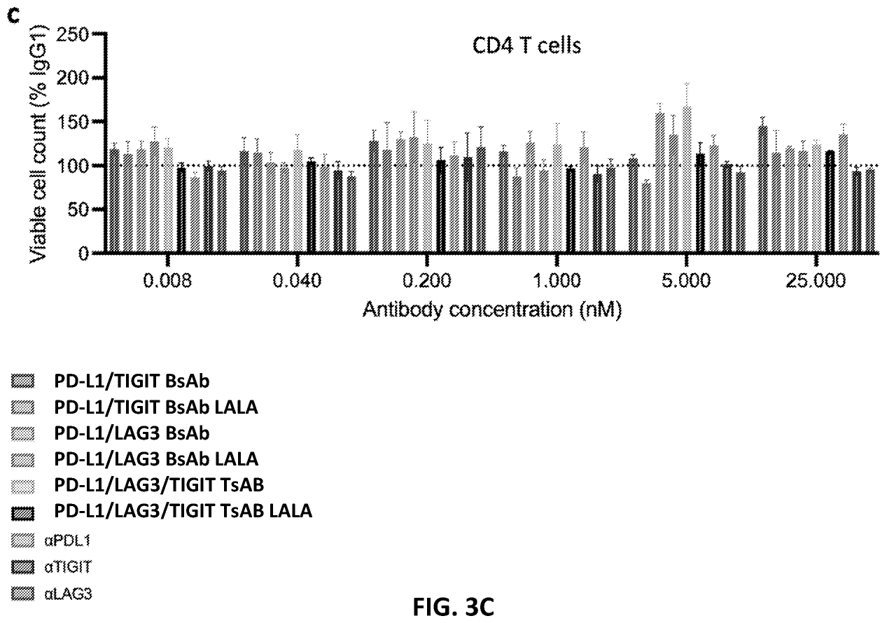

In co-culture of these cells (RKO OKT3) with human PBMCs, we found that PD-L1/TIGIT BsAb, PD-L1/LAG3 BsAb, and PD-L1/LAG3/TIGIT TsAb with wildtype IgG1 Fc outperformed parental antibodies in the promotion of cancer cell killing (FIG. 3A), CD8 T cell expansion (FIG. 3B), and CD4 T cell expansion (FIG. 3C). Fc binding to Fcγ receptors expressed on cells, such as monocyte/macrophages, NK cells, dendritic cells, and B cells, can have a profound impact on the efficacy of immune checkpoint-blocking antibodies. Although Fc-silencing is a common practice in the development of therapeutic antibodies targeting checkpoint molecules on T cells to prevent T cell depletion by ADCC/ADCP/CDC, we found that LALA (L234A/L235A) mutants of the three multispecific antibodies had greatly reduced potency (FIGS. 3A-3C), suggesting the importance of retaining Fc function for these antibodies. The results suggest that for therapeutic antibodies that engage T cells with tumor cells, intact Fc function combined with imbalanced binding affinities (relatively low T cell antigen binding and high tumor antigen binding) can effectively circumvent the issue of T cell depletion while potently promoting T cell activation and tumor cell killing.

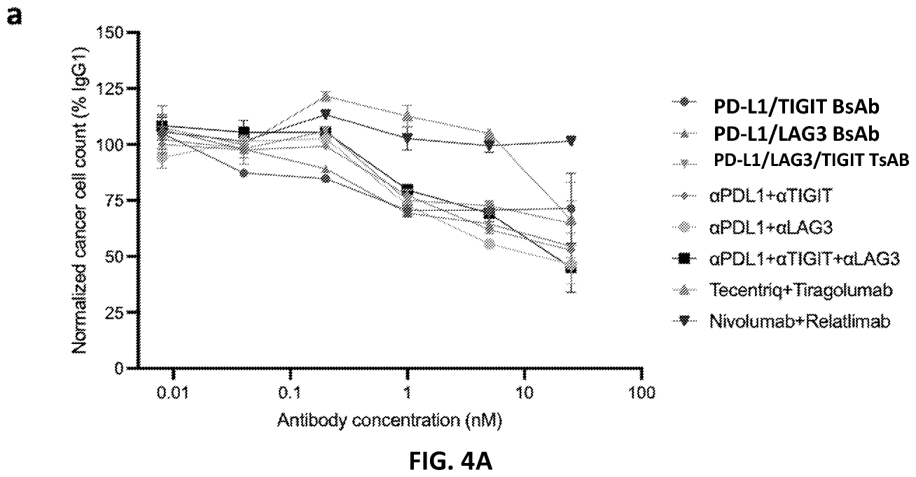

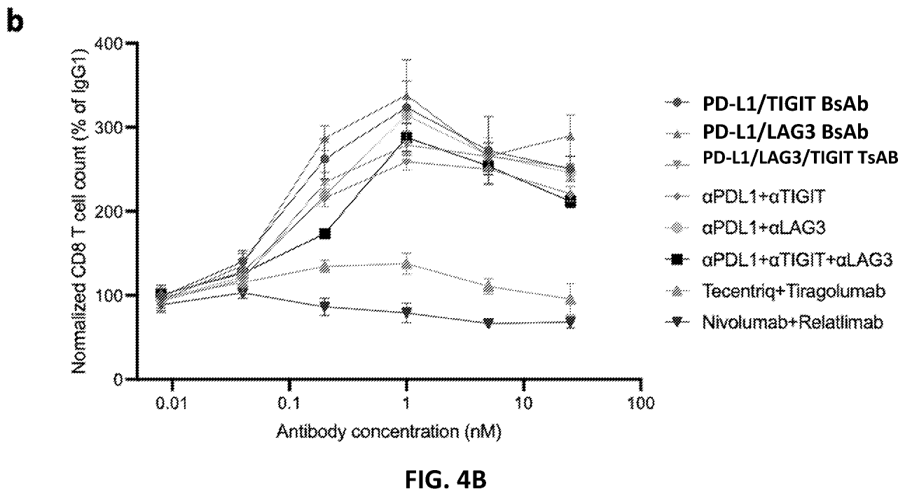

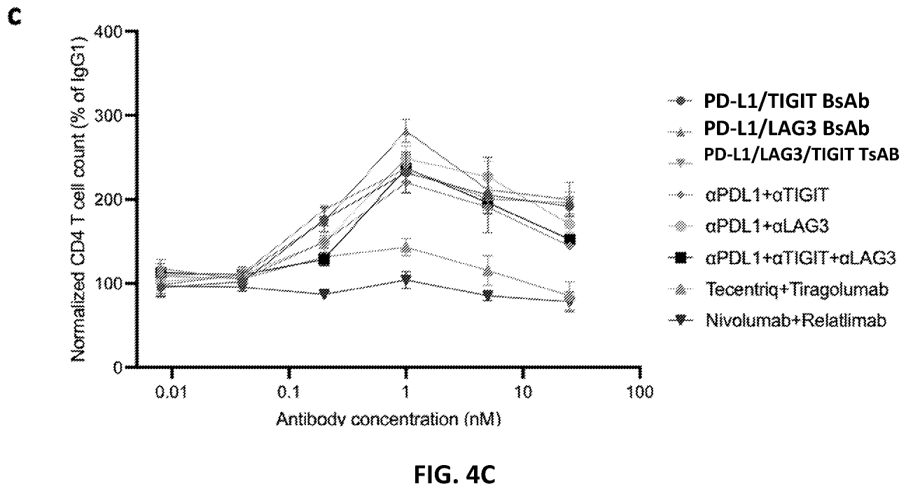

To test whether these antibodies are also efficacious with other cancer cells, we transfected the B cell lymphoma cell line Raji (FcγRIIB⁺PD-L1⁻MHC-II⁺CD155⁻) with OKT3, PD-L1, and CD155 and co-cultured the resultant cells (Raji OKT3 PDL1 CD155) with PBMCs in the presence of serial dilutions of test and control antibodies. We found that at antibody concentrations lower than 1 nM, our antibodies outperformed benchmark antibodies and parental antibody combinations in promotion of both cancer cell killing (FIG. 4A) and T cell expansion (FIGS. 4B-4C), correlating with cancer cell-T cell bridging (FIG. 2). At doses higher than 1 nM, they performed comparably with combinations of parental antibodies but better than benchmark antibodies, including nivolumab/relatlimab combination that has shown higher clinical activity than nivolumab monotherapy in untreated advanced melanoma. These data suggest in addition to intact Fc function, PD-L1/TIGIT BsAb, PD-L1/LAG3 BsAb, and PD-L1/LAG3/TIGIT TsAb-induced tumor cell-T cell bridging through PD-L1 on cancer cells and TIGIT/LAG-3 on T cells is a critical attribute for these antibodies to elicit efficient anti-cancer T cell response.

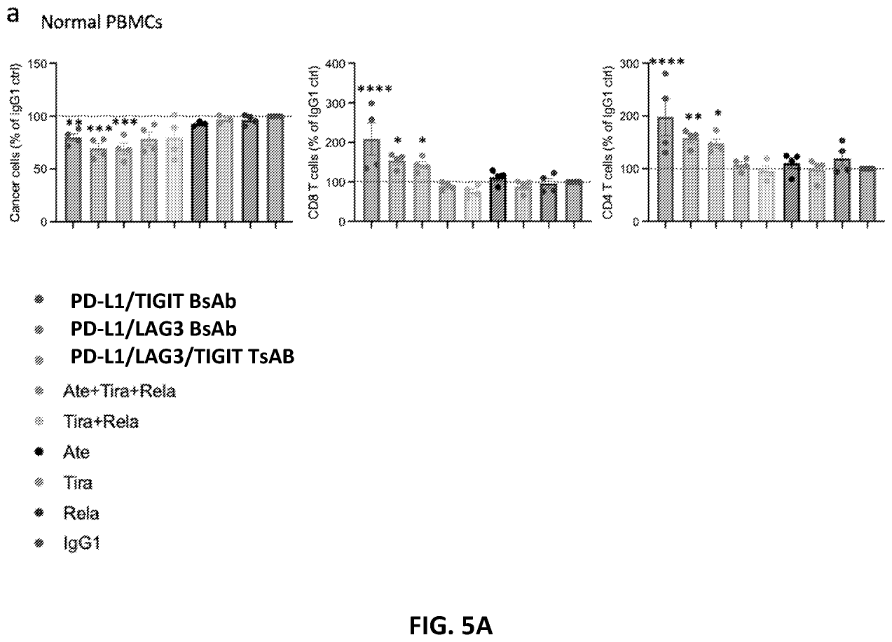

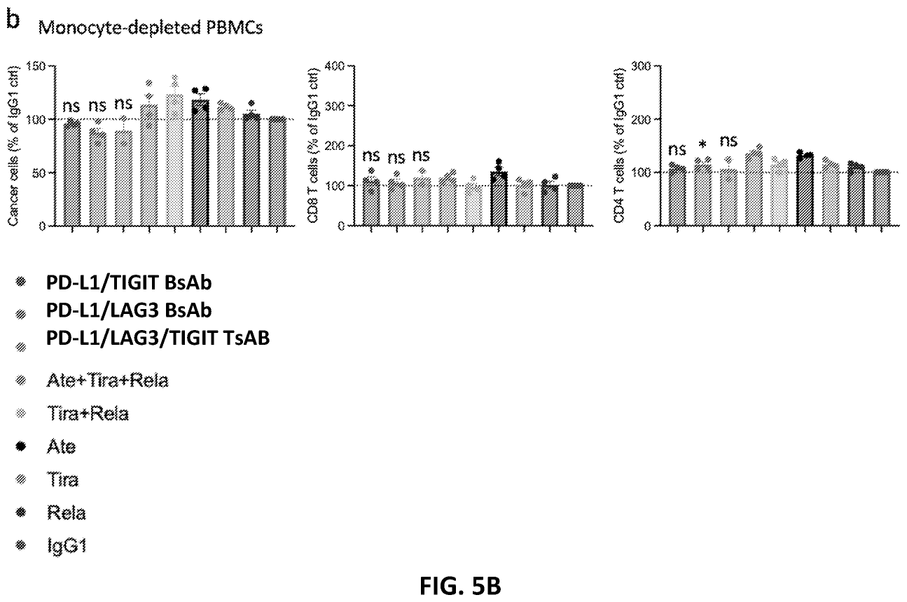

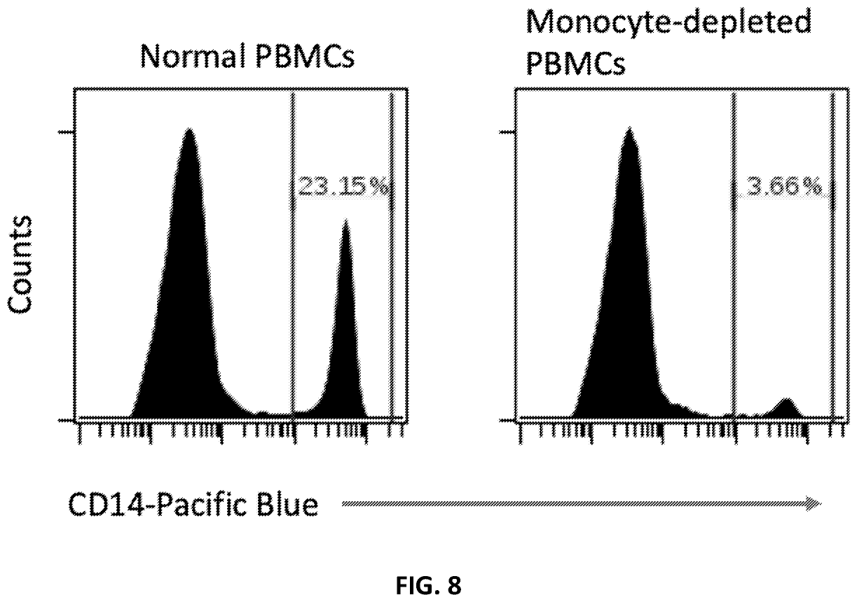

Fc binding to Fcγ receptors expressed on cells, such as monocyte/macrophages, NK cells, dendritic cells, and B cells, can have a profound impact on the efficacy of immune checkpoint-blocking antibodies. Although Fc-silencing is a common practice in the development of therapeutic antibodies targeting checkpoint molecules on T cells to prevent T cell depletion, FIG. 3C and FIG. 4 illustrate that wild-type Fc function in PD-L1/TIGIT BsAb, PD-L1/LAG3 BsAb, and PD-L1/LAG3/TIGIT TsAb actually enhances both T cell expansion and anti-tumor efficacy. We next tried to identify immune cells that mediate such effects. In PBMCs monocytes is a major component that express FcγRs and could contribute to the superior performance of Fc+PD-L1/TIGIT BsAb, PD-L1/LAG3 BsAb, and PD-L1/LAG3/TIGIT TsAb. To confirm this, we compared antibody efficacy in co-cultures of cancer cells (Raji PD-L1 OKT3) with either normal PBMCs or monocyte-depleted PBMCs (FIGS. 5A-5B; FIG. 8). We found that while PD-L1/TIGIT BsAb, PD-L1/LAG3 BsAb, and PD-L1/LAG3/TIGIT TsAb promoted greater T cell expansion than control IgG1 and monospecific antibody combinations in co-culture of T cells with normal PBMCs (FIG. 5A), their activities were largely mitigated when monocyte-depleted PBMCs were used in the co-culture (FIG. 5B). These results suggest the importance of monocytes for the efficacy of PD-L1/TIGIT BsAb, PD-L1/LAG3 BsAb, and PD-L1/LAG3/TIGIT TsAb. This is likely because these myeloid cells express multiple proteins that can mediate the function of these antibodies in T cell activation, such as FcγRs, PD-L1, CD155, MHC-II and co-stimulatory molecules that include CD80 and CD86.

Taken together, the in vitro data demonstrate that Fc-competent PD-L1/TIGIT BsAb, PD-L1/LAG3 BsAb, and PD-L1/LAG3/TIGIT TsAb are highly efficient in promoting T cell expansion and cancer cell killing when compared with benchmark antibody and antibody combinations through multiple MOAs including checkpoint blockade, Fc-mediated functions, and cancer cell-T cell bridging.

### Example 4. PD-L1/TIGIT BsAb and PD-L1/LAG3/TIGIT TsAb Exhibit Superior Tumor Suppression Activity in Animal Models

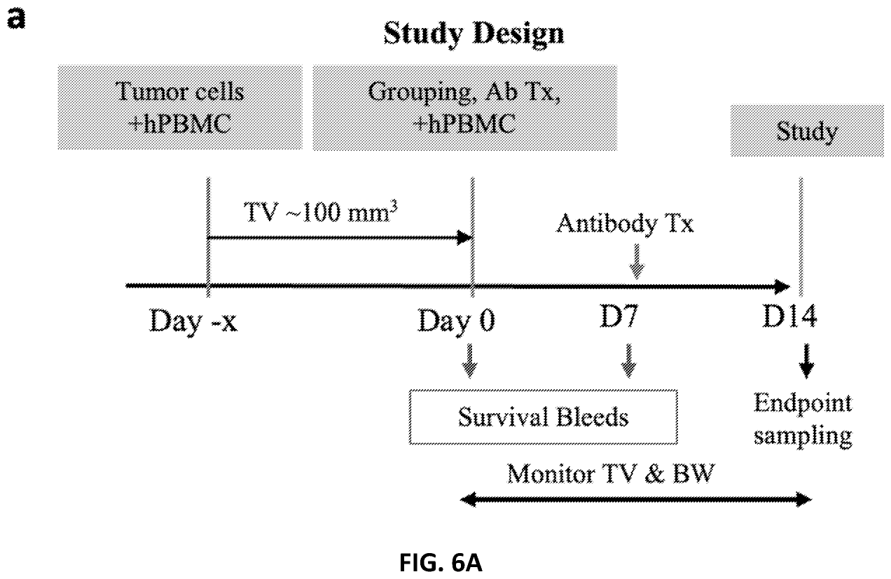

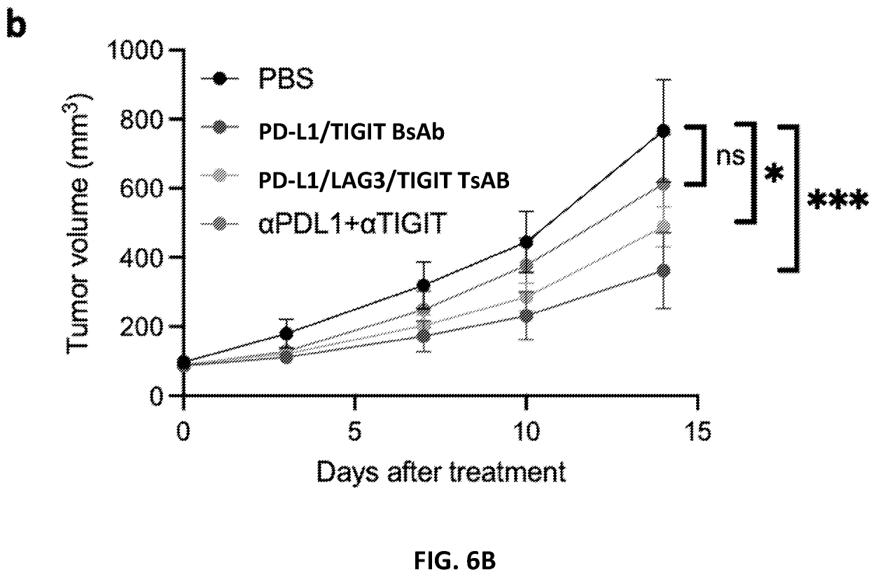

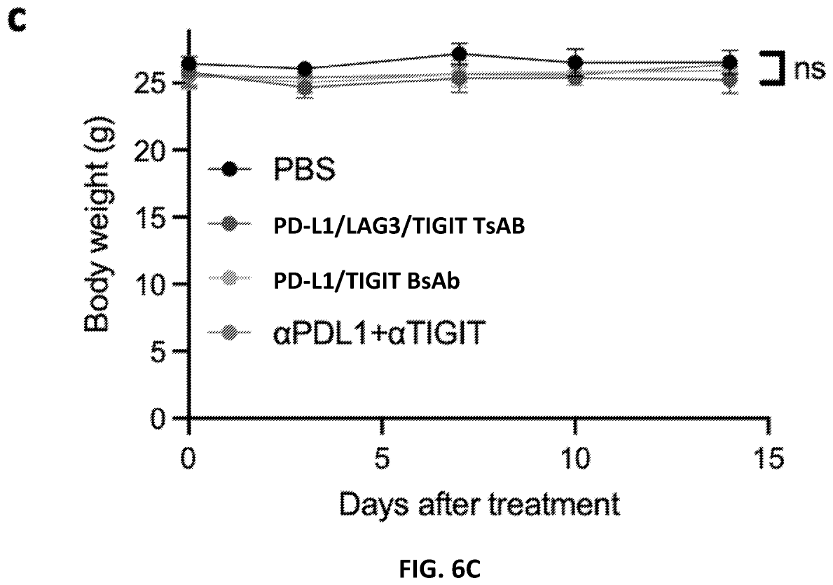

Encouraged by the results from in vitro cell-based assays, we further evaluated the antibodies in animal models. RKO OKT3 human colon cancer cells and human PBMCs were co-engrafted into NSG (NOD-scid IL2Rg^{null}) immunodeficient mice, and the mice were subjected to antibody treatment after tumors were established. Tumor volume as well as body weight were monitored thereafter to evaluate antibody efficacy and toxicity, respectively (FIG. 6A). We found that both PD-L1/TIGIT BsAb and PD-L1/LAG3/TIGIT TsAb, but not the anti-PDL1/anti-TIGIT combination, elicited significant tumor growth suppression (FIG. 6B), with no significant effects on body weight (FIG. 6C). The results demonstrate the antitumor efficacy of PD-L1/TIGIT BsAb and PD-L1/LAG3/TIGIT TsAb in vivo with no appreciable safety concerns. We also monitored mouse serum levels of human antibodies at different time points after administration and found that PD-L1/LAG3/TIGIT TsAb had a shorter half-life than PD-L1/TIGIT BsAb or anti-PDL1/anti-TIGIT combination.

### Example 5. Key Assay Systems for Functional Evaluation of Antibodies

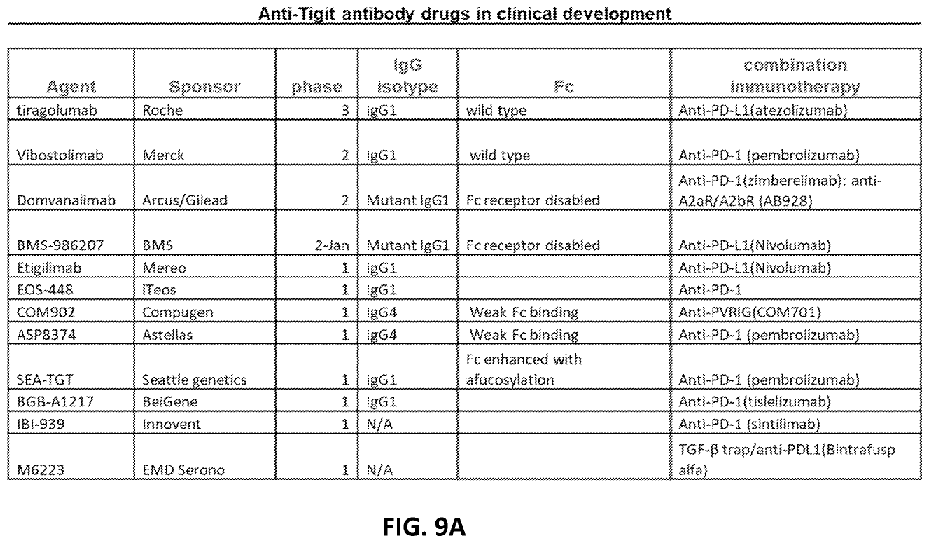

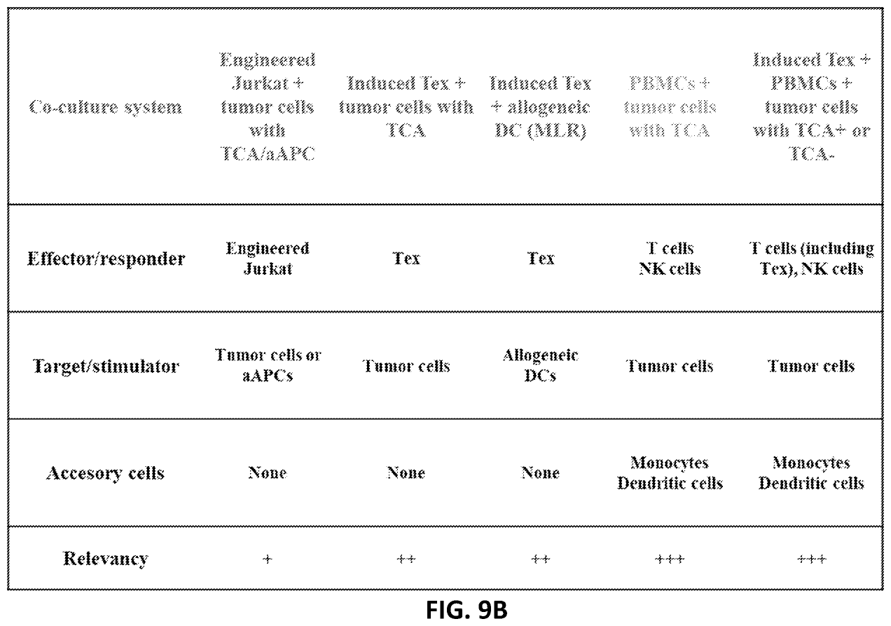

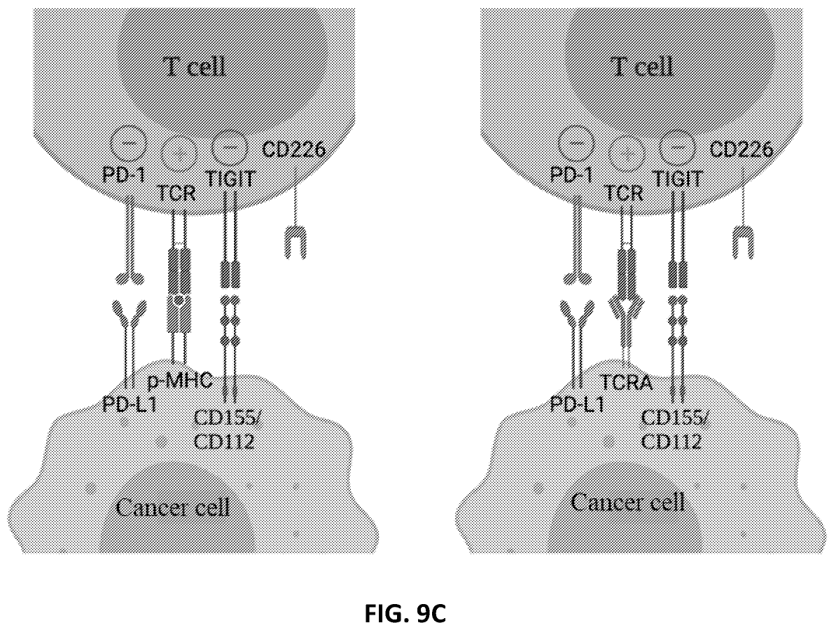

Evidence suggests that a wild-type IgG1 isotype can kill antibody-coated target cells by ADCC. This function can facilitate depletion of immuno-suppressive Treg cells that express high levels of TIGIT and compromise antitumor immunity. However, mutated IgG1 tails with inactivated Fe regions have also been used to avoid potential risk of T effector cell loss. According to the Fc identity of current anti-TIGIT antibody drugs in clinical development shown in FIG. 9A, the data suggests that retaining Fc function is beneficial.

As shown in FIGS. 9B-9C, the core components of the assay systems described herein mimicked the cellular context of checkpoint regulation of T cell activation in vivo. All relevant molecules were naturally expressed except that an activator of the TCR complex (TCRA) was engineered on tumor cell-surface to activate the T cell in an antigen-independent manner.

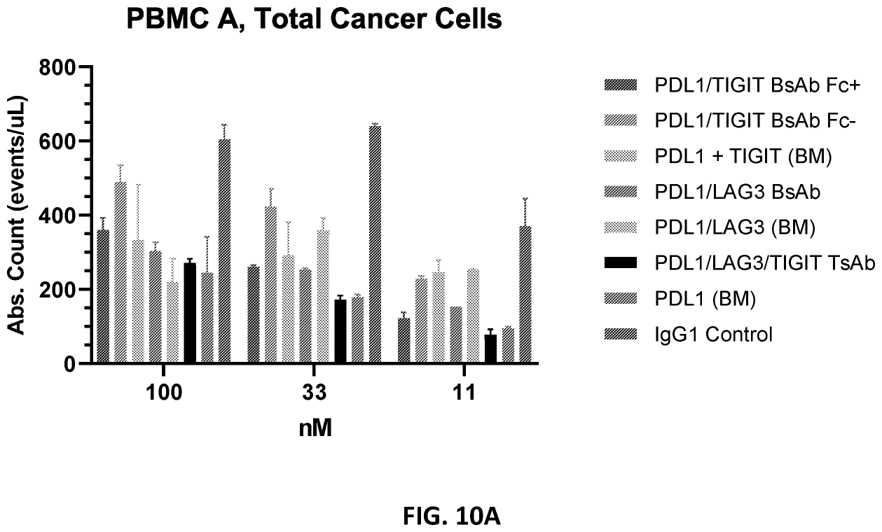

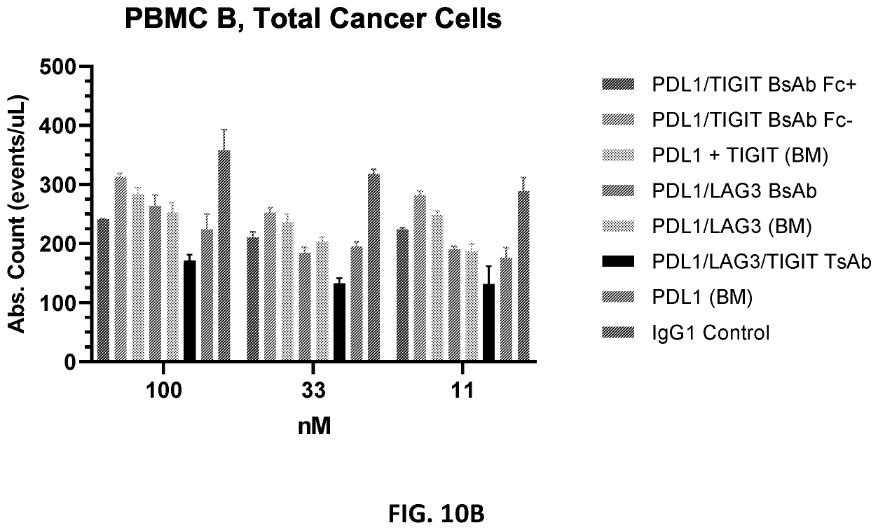

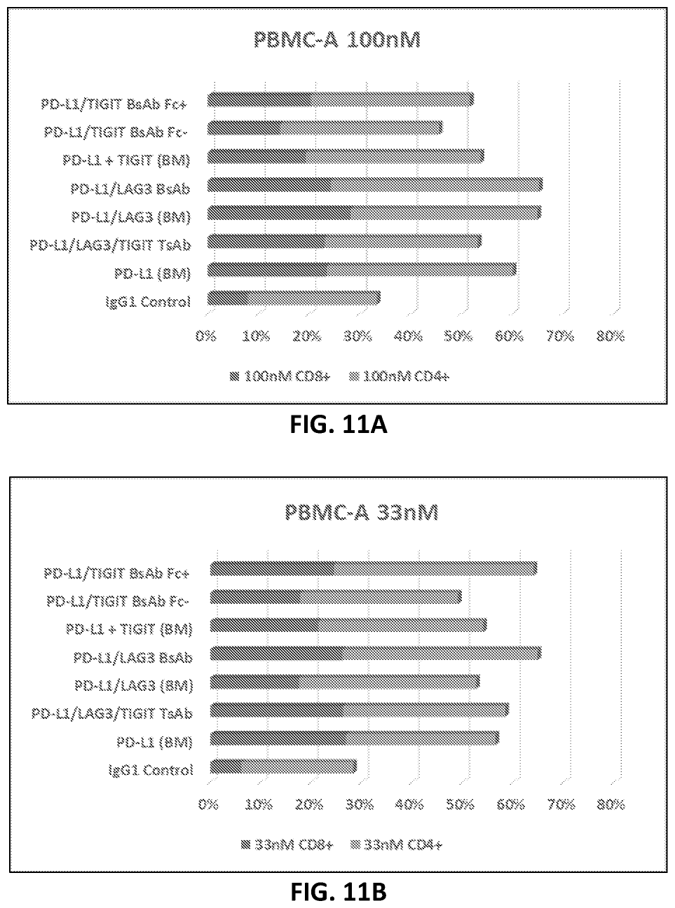

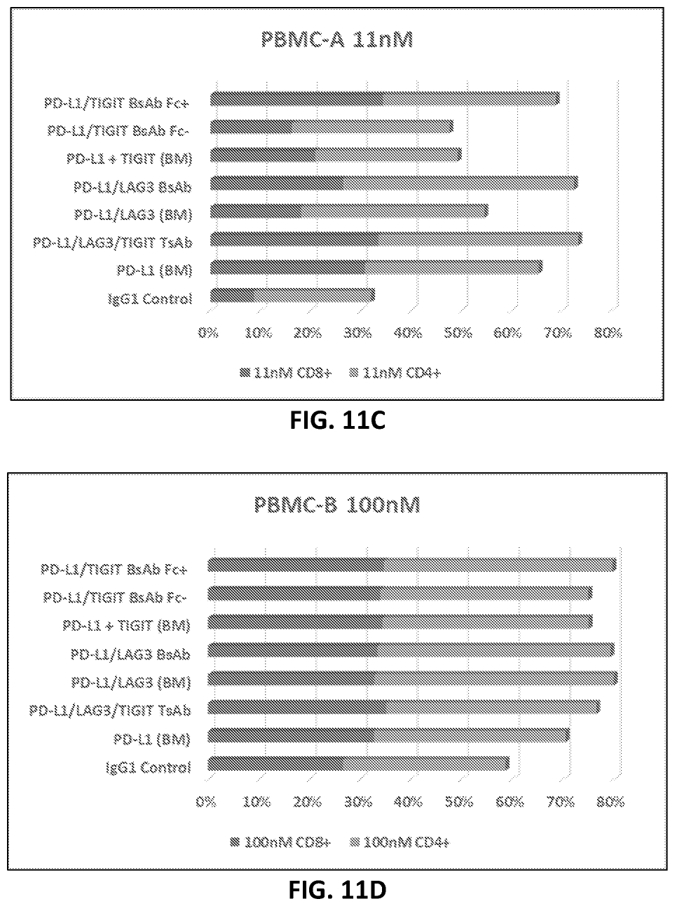

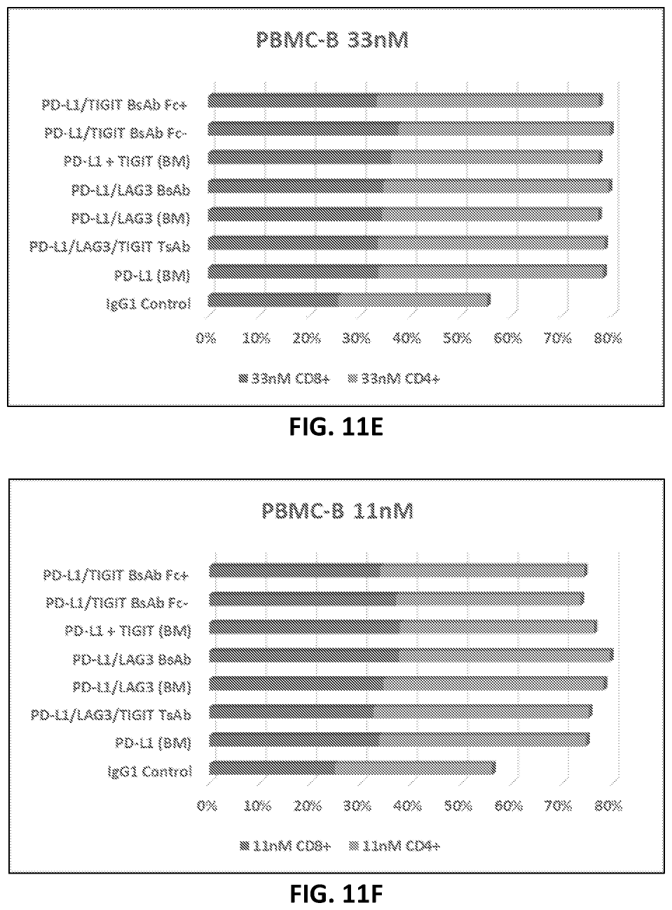

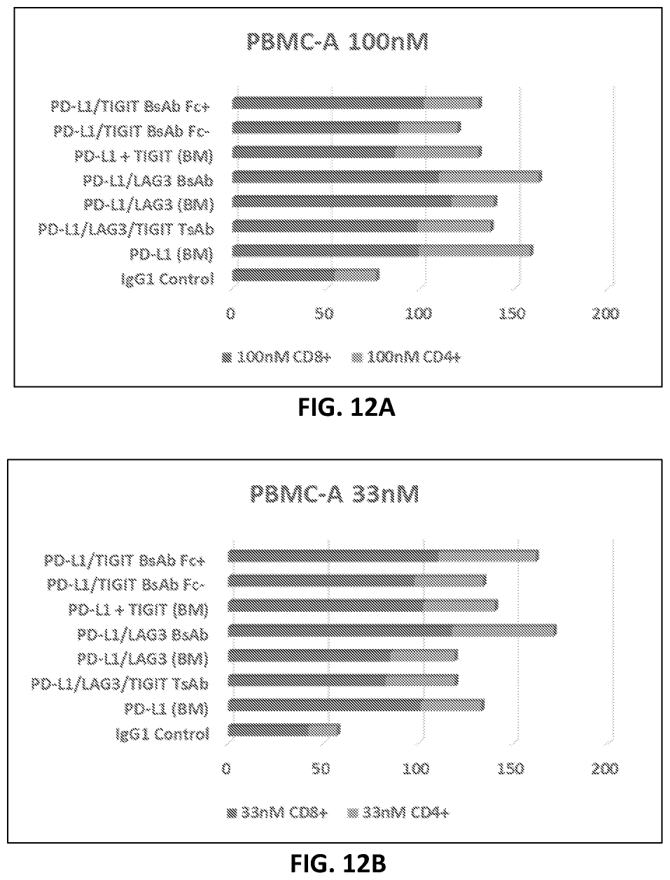

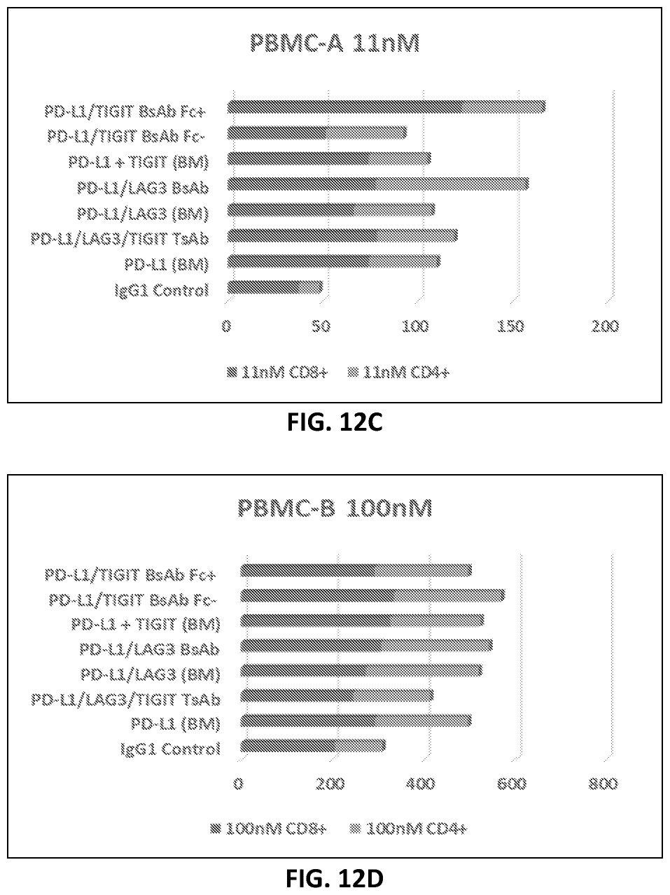

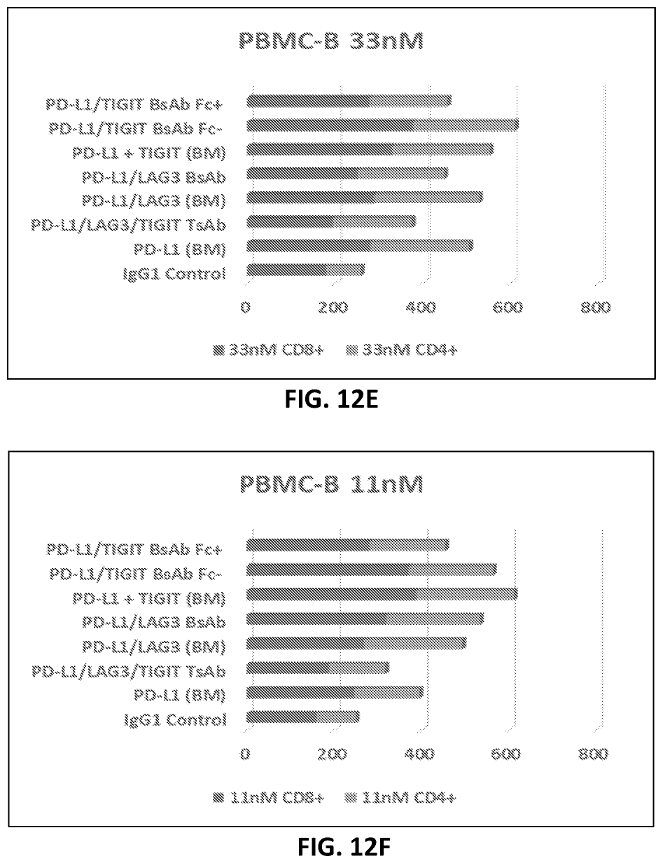

Therapeutic antibodies were evaluated by co-culturing with PBMCs (high monocyte content-PBMC-A or low monocyte content-PBMC-B) and cancer cells. Specifically, cancer cells expressing a T cell activator (TCA) were co-cultured with PBMCs at a ratio of 1:5 for 4 days. Numbers of viable cancer cells, CD8+ and CD4+ T cells were then determined by flow cytometry. Absolute numbers for cancer cells were determined, and the results are shown in FIGS. 10A-10B. T cell profiling of CD8+ and CD4+ cells (percentage) was performed, and the results are shown in FIGS. 11A-11F. Absolute numbers of T cell proliferation are shown in FIGS. 12A-12F. According to the results, the PD-L1/LAG3/TIGIT TsAb (tri-specific antibody) was found to be the most effective in cancer cell killing as well as increasing CD8+ and CD4+ T cell proliferation.

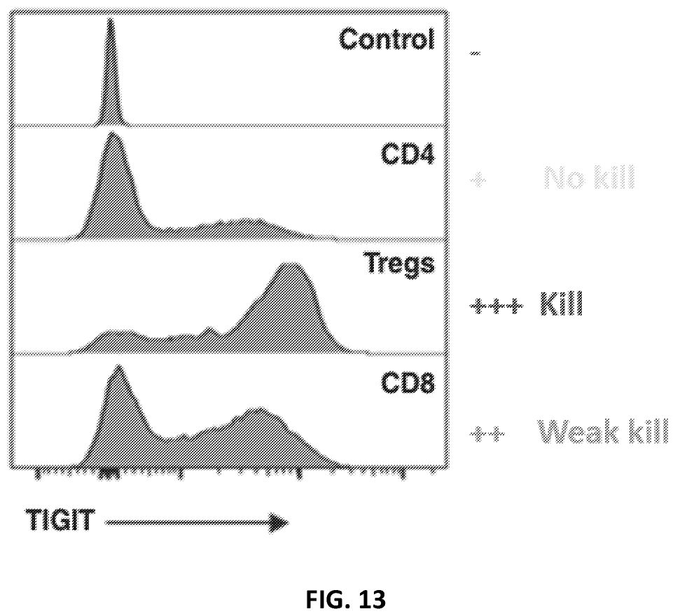

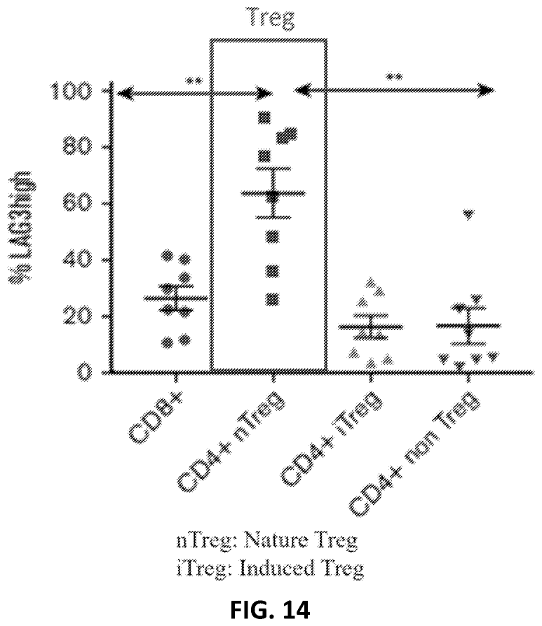

As shown in FIG. 13, TIGIT overexpressed on Tregs and CD8+ T cell. As shown in FIG. 14, LAG3 also overexpressed on Tregs.

In summary, Fc effector function of the PD-L1/TIGIT BsAb (bispecific antibody) induced more cancer cell killing in the presence of more monocytes. In addition, Fc effector function did not affect the function of the PD-L1/LAG3 BsAb. PD-L1/LAG3 BsAb induced similar or higher conventional CD4+ T cell expansion compared to that of a Fc silenced benchmark. Further, the PD-L1/LAG3/TIGIT TsAb showed better cancer killing than bispecific antibodies. No significant side effect of Fc effector function was observed.

### Example 6. Donor PBMC+Exhausting T Cell+Cancer Cell Co-Culture Assay

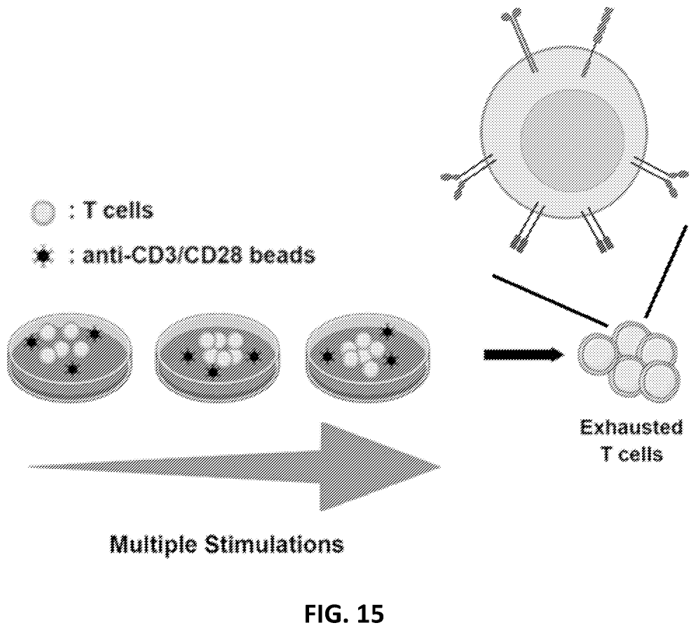

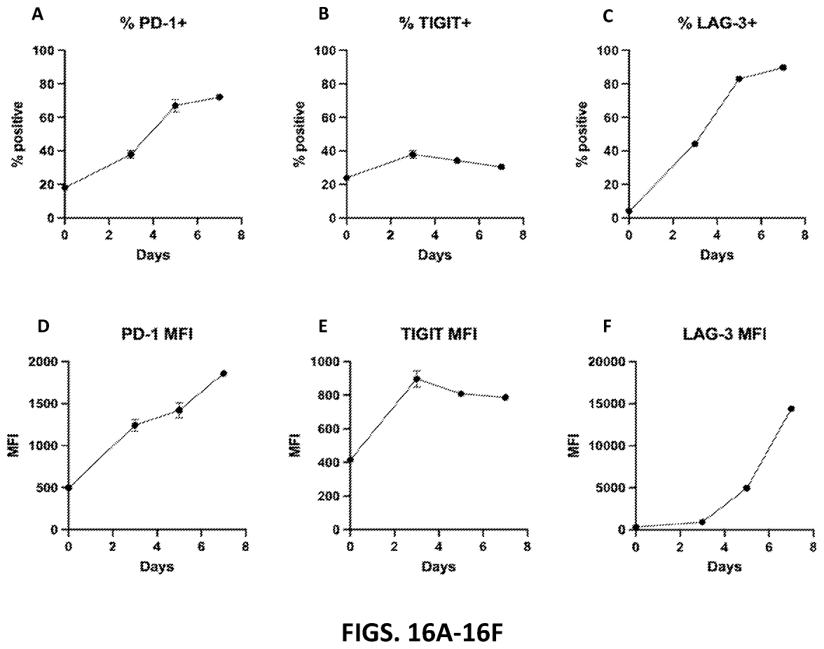

T cell exhaustion was induced by repeated T cell receptor stimulations, as shown in FIG. 15. Human peripheral blood mononuclear cells (PBMCs) were stimulated for 3 days with T-Activator CD3/CD28 Dynabeads™ (Thermo). After stimulation, the PBMCs were washed, and restimulated twice (2 days each) with a fresh batch of beads. Expression of the T cell exhaustion markers PD-1, TIGIT, and LAG-3 were monitored at indicated time points using flow cytometry. Positive cell percentages and MFI (mean fluorescence intensity) values are shown for each marker in FIGS. 16A-16C and FIGS. 16D-16F, respectively.

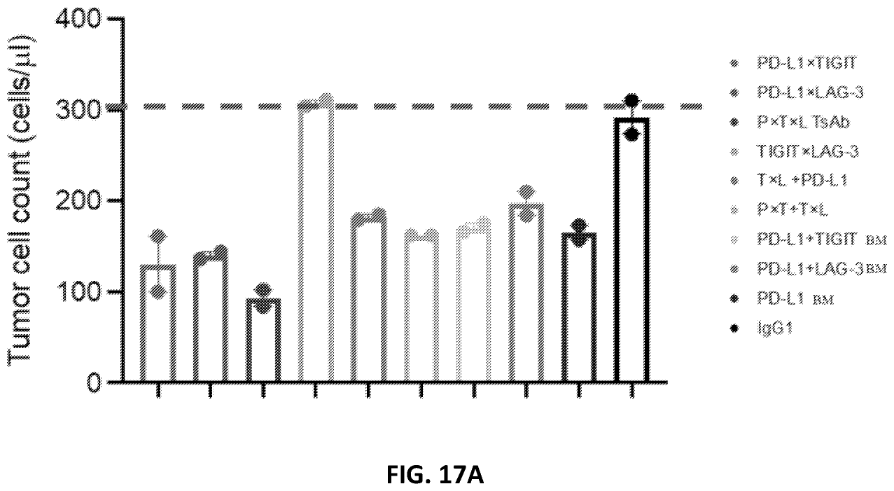

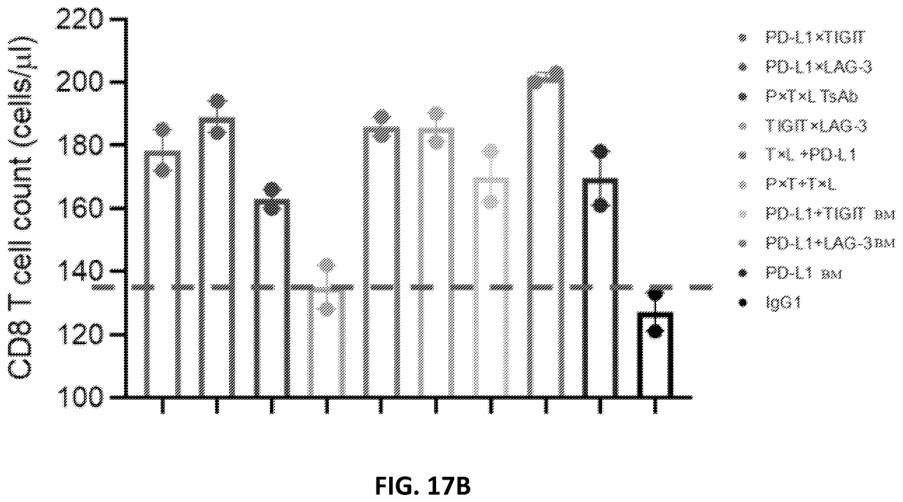

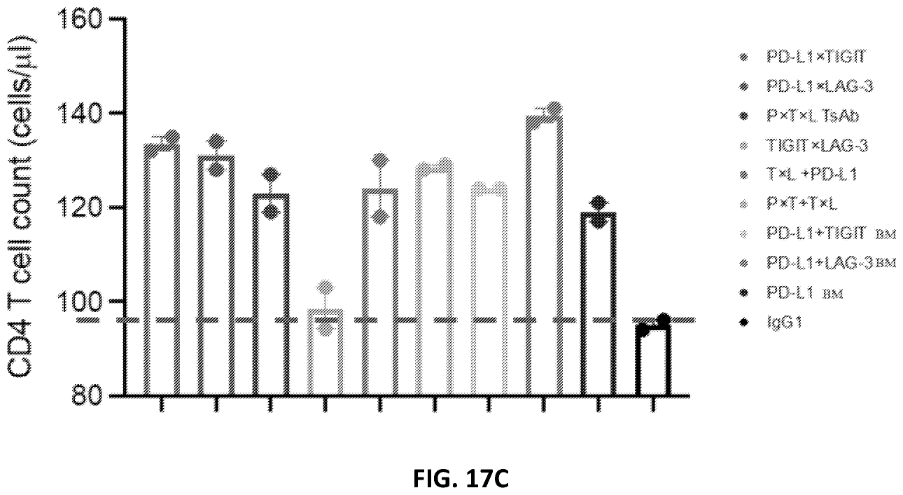

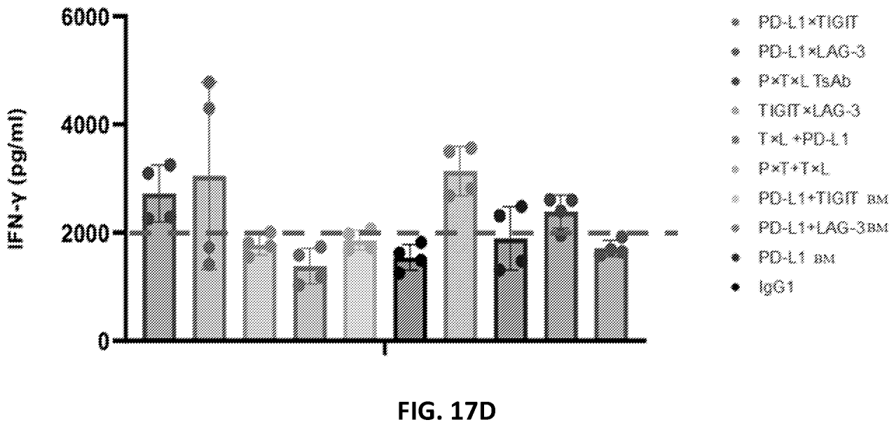

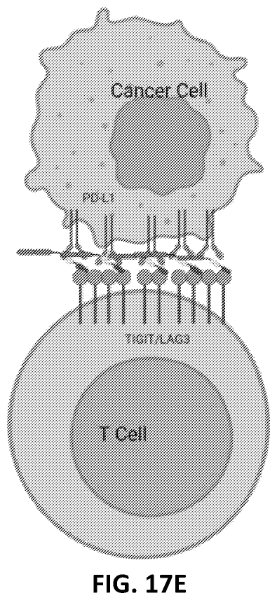

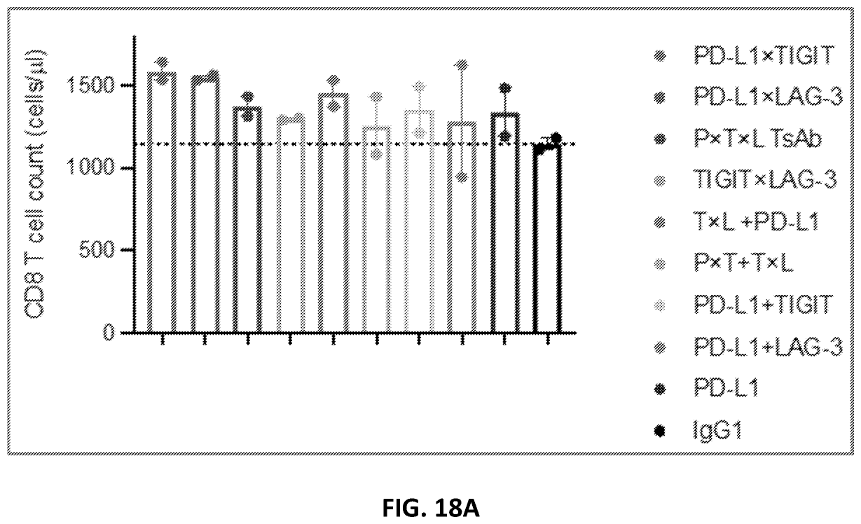

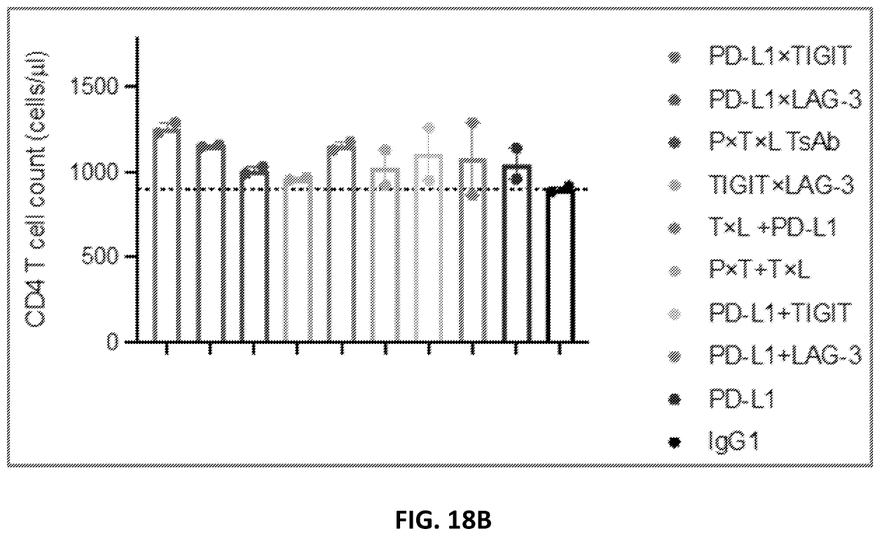

Therapeutic antibodies were evaluated by co-culturing of PBMCs/exhausted T cells with cancer cells or allogeneic dendritic cells (DCs). Colon cancer cells (PD-L1+, CD155+, MHC II−) were stably transfected to express a T cell activator (TCA). The cancer cells were co-cultured with PBMCs and exhausted T cells at a ratio of 1:2.5:2.5 for 4 days. As shown in FIGS. 17A-17D, numbers of viable cancer cells, CD8 and CD4 T cells were then determined by flow cytometry, and IFN-γ levels by ELISA. FIG. 17E is a schematic diagram illustrating the interaction between cancer cells and T cells. Further, mixed lymphocyte reaction (MLR) assays were performed with exhausted T cells and allogeneic monocyte-derived dendritic cells at a ratio of 10:1 for 5 days. As shown in FIGS. 18A-18B, numbers of viable CD8+ and CD4+ T cells were determined by flow cytometry.

In conclusion, when T cells were induced to be exhausted first, followed by mixing with PBMCs, the tested BsAbs and TsAbs induced better cancer killing than that of PD-L1 Mab (monoclonal antibody) and its combinations with other immune checkpoint inhibitors. In addition, the tested BsAbs and TsAbs performed similarly in MLR system.

### Example 7. Animal Study to Investigate Tumor Growth and TILs

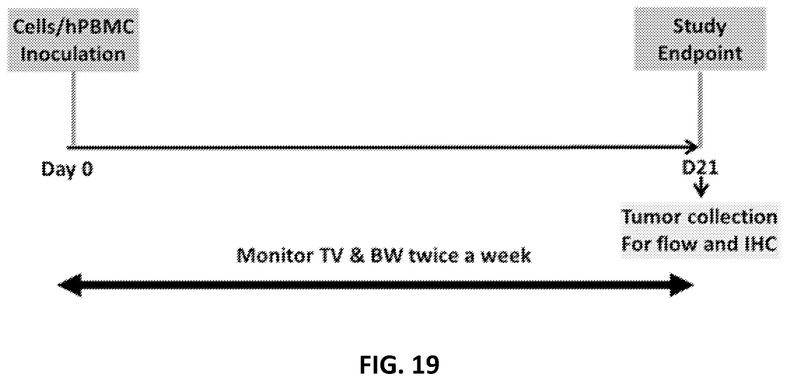

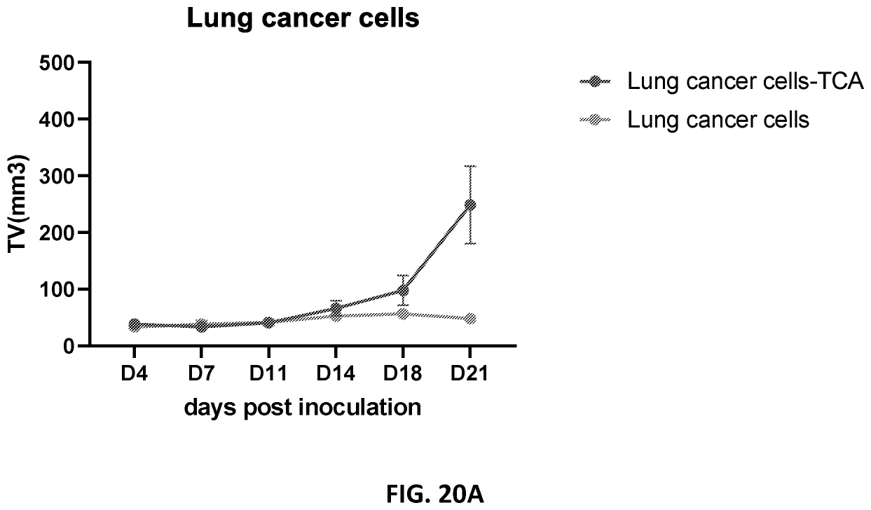

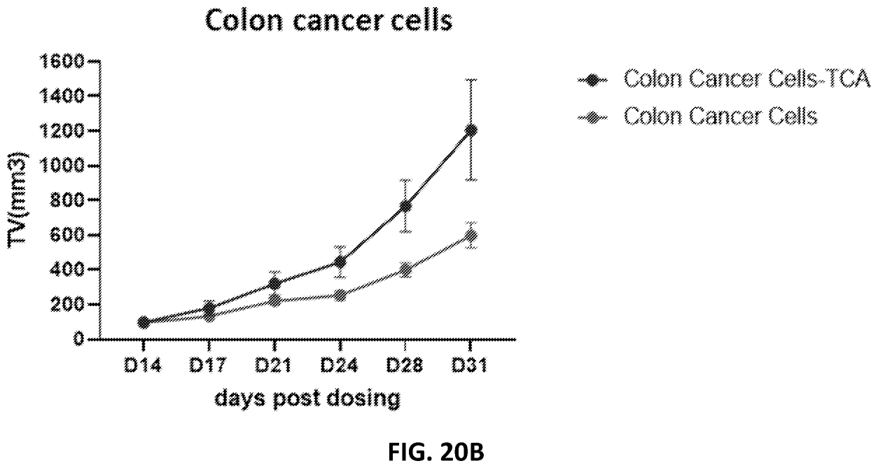

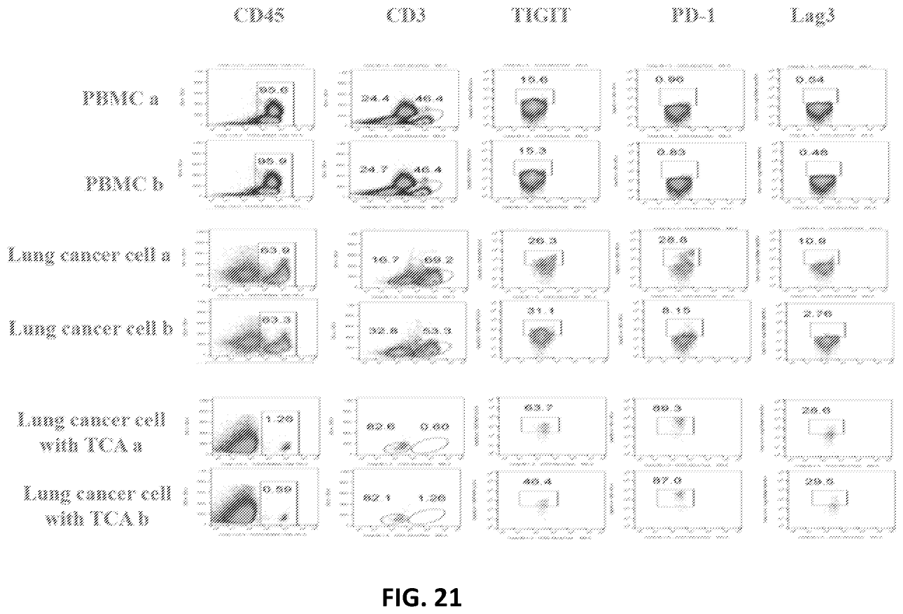

An animal model for T cell exhaustion study was developed as follows. Cancer-TCA cells were co-inoculated with human PBMCs into NSG™ mice. Tumor sizes were measured twice a week for 21 days, at which point tumors were collected. The study design is shown in FIG. 19. The tumor volume (TV) curves of lung cancer and colon cancer development are shown in FIGS. 20A-20B. As shown in FIG. 21, cancer and T cell numbers, and T cell exhausting status were analyzed by FACS.

The results suggest increased growth rate of cancer-TCA cells compared to parental cancer cells. T cell exhaustion was observed. Moreover, expression of LAG3, TIGIT, and PD-1 were increased.

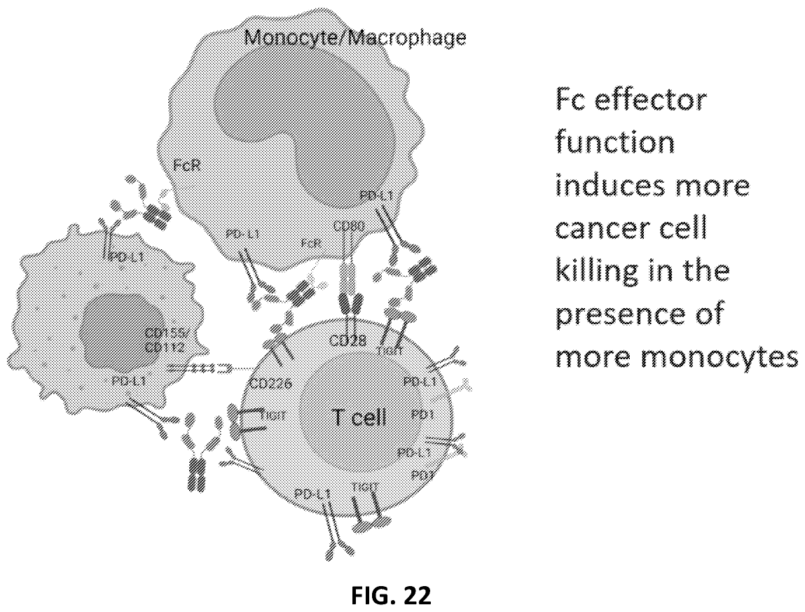

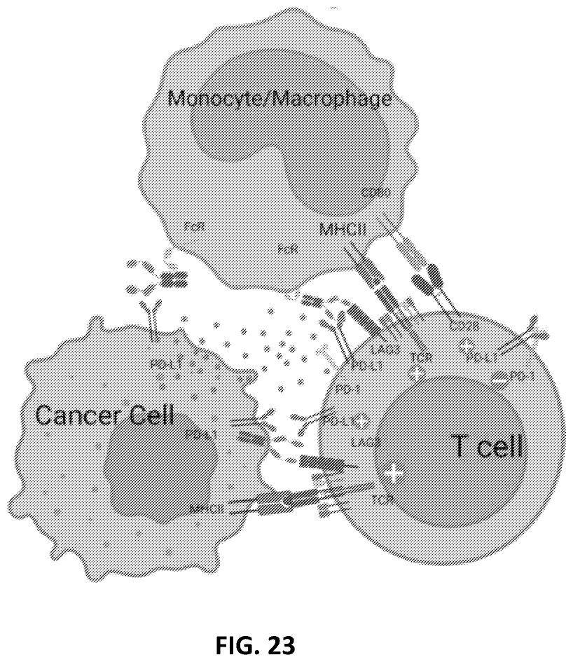

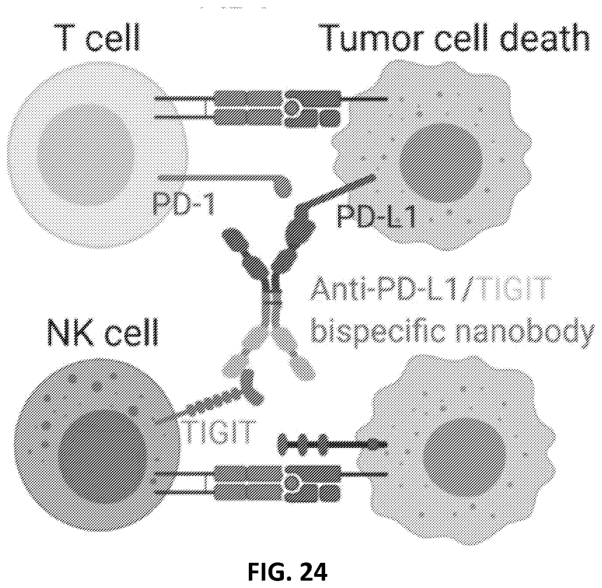

In summary, PD-L1/TIGIT bispecific antibodies with Fc function retained (Fc+) performed better than PD-L1/TIGIT bispecific antibodies with Fc function silenced (Fc−), in the presence of high numbers of monocytes in the co-culture. PD-L1/TIGIT-Fc+BsAb performed better than the combination of anti-PD-L1 and anti-TIGIT benchmark antibodies. The PD-L1/LAG3/TIGIT TsAb induced cancer cell killing under all testing conditions in cell-based assays. Both of the PD-L1/TIGIT BsAb and the PD-L1/LAG3/TIGIT TsAb suppressed tumor growth better than the combination of anti-PD-L1 and anti-TIGIT benchmark antibodies in the T cell exhausting animal model. FIG. 22 and FIG. 23 show cartoons of PD-L1/TIGIT BsAb and FIG. 24 shows a cartoon of PD-L1/LAG3 BsAb interacting with cells.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to LAG3 (Lymphocyte-activation gene 3), comprising:
a heavy-chain antibody variable domain (VHH) comprising complementarity determining regions (CDRs) 1, 2, and 3, wherein the VHH CDR1 region comprises an amino acid sequence that is at least 80% identical to a selected VHH CDR1 amino acid sequence, the VHH CDR2 region comprises an amino acid sequence that is at least 80% identical to a selected VHH CDR2 amino acid sequence, and the VHH CDR3 region comprises an amino acid sequence that is at least 80% identical to a selected VHH CDR3 amino acid sequence;
wherein the selected VHH CDRs 1, 2, and 3 amino acid sequences are one of the following:
(1) the selected VHH CDRs 1, 2, 3 amino acid sequences are set forth in SEQ ID NOs: 1, 2, and 3, respectively;
(2) the selected VHH CDRs 1, 2, 3 amino acid sequences are set forth in SEQ ID NOs: 4, 5, and 6, respectively;
(3) the selected VHH CDRs 1, 2, 3 amino acid sequences are set forth in SEQ ID NOs: 7, 8, and 9, respectively;
(4) the selected VHH CDRs 1, 2, 3 amino acid sequences are set forth in SEQ ID NOs: 10, 11, and 12, respectively; and
(5) the selected VHH CDRs 1, 2, 3 amino acid sequences are set forth in SEQ ID NOs: 13, 14, and 15, respectively;

2. The antibody or antigen-binding fragment thereof of claim 1, wherein the VHH comprises CDRs 1, 2, 3 with the amino acid sequences set forth in SEQ ID NOs: 1, 2, and 3, respectively.

3. The antibody or antigen-binding fragment thereof of claim 1, wherein the VHH comprises CDRs 1, 2, 3 with the amino acid sequences set forth in SEQ ID NOs: 4, 5, and 6, respectively.

4. The antibody or antigen-binding fragment thereof of claim 1, wherein the VHH comprises CDRs 1, 2, 3 with the amino acid sequences set forth in SEQ ID NOs: 7, 8, and 9, respectively.

5. The antibody or antigen-binding fragment thereof of claim 1, wherein the VHH comprises CDRs 1, 2, 3 with the amino acid sequences set forth in SEQ ID NOs: 10, 11, and 12, respectively.

6. The antibody or antigen-binding fragment thereof of claim 1, wherein the VHH comprises CDRs 1, 2, 3 with the amino acid sequences set forth in SEQ ID NOs: 13, 14, and 15, respectively.

7. An antibody or antigen-binding fragment thereof that binds to LAG3 comprising a heavy-chain antibody variable domain (VHH) comprising an amino acid sequence that is at least 80% identical to a selected VHH sequence, wherein the selected VHH sequence is selected from the group consisting of SEQ ID NOs: 22-26.

8. The antibody or antigen-binding fragment thereof of any one of claims 1-7, wherein the antibody or antigen-binding fragment specifically binds to human LAG3.

9. An antibody or antigen-binding fragment thereof that binds to TIGIT (T cell immunoreceptor with Ig and ITIM domains), comprising:
a heavy-chain antibody variable domain (VHH) comprising complementarity determining regions (CDRs) 1, 2, and 3, wherein the VHH CDR1 region comprises an amino acid sequence that is at least 80% identical to SEQ ID NO: 16, the VHH CDR2 region comprises an amino acid sequence that is at least 80% identical to SEQ ID NO: 17, and the VHH CDR3 region comprises an amino acid sequence that is at least 80% identical to SEQ ID NO: 18.

10. The antibody or antigen-binding fragment thereof of claim 9, wherein the VHH comprises CDRs 1, 2, 3 with the amino acid sequences set forth in SEQ ID NOs: 16, 17, and 18, respectively.

11. An antibody or antigen-binding fragment thereof that binds to TIGIT comprising a heavy-chain antibody variable domain (VHH) comprising an amino acid sequence that is at least 80% identical to SEQ ID NO: 27.

12. The antibody or antigen-binding fragment thereof of any one of claims 9-11, wherein the antibody or antigen-binding fragment specifically binds to human TIGIT.

13. An antibody or antigen-binding fragment thereof that binds to PD-L1 (Programmed death-ligand 1), comprising:
a heavy-chain antibody variable domain (VHH) comprising complementarity determining regions (CDRs) 1, 2, and 3, wherein the VHH CDR1 region comprises an amino acid sequence that is at least 80% identical to SEQ ID NO: 19, the VHH CDR2 region comprises an amino acid sequence that is at least 80% identical to SEQ ID NO: 20, and the VHH CDR3 region comprises an amino acid sequence that is at least 80% identical to SEQ ID NO: 21.

14. The antibody or antigen-binding fragment thereof of claim 9, wherein the VHH comprises CDRs 1, 2, 3 with the amino acid sequences set forth in SEQ ID NOs: 19, 20, and 21, respectively.

15. An antibody or antigen-binding fragment thereof that binds to PD-L1 comprising a heavy-chain antibody variable domain (VHH) comprising an amino acid sequence that is at least 80% identical to SEQ ID NO: 28.

16. The antibody or antigen-binding fragment thereof of any one of claims 13-15, wherein the antibody or antigen-binding fragment specifically binds to human PD-L1.

17. The antibody or antigen-binding fragment thereof of any one of claims 1-16, wherein the antibody or antigen-binding fragment is a humanized antibody or antigen-binding fragment thereof.

18. An antibody or antigen-binding fragment thereof comprising the VHH CDRs 1, 2, 3, of the antibody or antigen-binding fragment thereof of any one of claims 1-17.

19. The antibody or antigen-binding fragment thereof of any one of claims 1-18, wherein the antibody or antigen-binding fragment comprises a human IgG Fc (e.g., a human IgG Fc comprising an amino acid sequence that is at least 80% identical to SEQ ID NO: 29).

20. The antibody or antigen-binding fragment thereof of any one of claims 1-19, wherein the antibody or antigen-binding fragment comprises two or more heavy-chain antibody variable domains.

21. An antibody or antigen-binding fragment thereof that cross-competes with the antibody or antigen-binding fragment thereof of any one of claims 1-20.

22. The antibody or antigen-binding fragment thereof of any one of claims 1-21, wherein the antibody or antigen-binding fragment is a single-chain variable fragment (scFv) or a multispecific antibody (e.g., a bispecific antibody).

23. A multi-specific antibody or antigen-binding fragment thereof, comprising a first antigen-binding site that specifically binds to PD-L1, and a second antigen-binding site that specifically binds to TIGIT.

24. The multi-specific antibody or antigen-binding fragment thereof of claim 23, wherein the first antigen-binding site comprises a first heavy-chain antibody variable domain (VHH1) that specifically binds to PD-L1, wherein
the VHH1 comprises complementarity determining regions (CDRs) 1, 2, and 3, wherein the VHH1 CDR1 region comprises an amino acid sequence that is at least 80% identical to SEQ ID NO: 19, the VHH1 CDR2 region comprises an amino acid sequence that is at least 80% identical to SEQ ID NO: 20, and the VHH1 CDR3 region comprises an amino acid sequence that is at least 80% identical to SEQ ID NO: 21.

25. The multi-specific antibody or antigen-binding fragment thereof of claim 24, wherein the VHH1 comprises CDRs 1, 2, 3 with the amino acid sequences set forth in SEQ ID NOs: 19, 20, and 21, respectively.

26. The multi-specific antibody or antigen-binding fragment thereof of claim 25, wherein the VHH1 comprises an amino acid sequence that is at least 80% identical to SEQ ID NO: 28.

27. The multi-specific antibody or antigen-binding fragment thereof of any one of claims 23-26, wherein the second antigen-binding site specifically binds to TIGIT, and the second antigen-binding site comprises a second heavy-chain antibody variable domain (VHH2) that specifically binds to TIGIT, wherein
the VHH2 comprises complementarity determining regions (CDRs) 1, 2, and 3, wherein the VHH2 CDR1 region comprises an amino acid sequence that is at least 80% identical to SEQ ID NO: 16, the VHH2 CDR2 region comprises an amino acid sequence that is at least 80% identical to SEQ ID NO: 17, and the VHH2 CDR3 region comprises an amino acid sequence that is at least 80% identical to SEQ ID NO: 18.

28. The multi-specific antibody or antigen-binding fragment thereof of claim 27, wherein the VHH2 comprises an amino acid sequence that is at least 80% identical to SEQ ID NO: 27.

29. The multi-specific antibody or antigen-binding fragment thereof of any one of claims 23-38, further comprising a third antigen-binding site that specifically binds to LAG3.

30. The multi-specific antibody or antigen-binding fragment thereof of claim 29, wherein the third antigen-binding site specifically binds to LAG3, and the third antigen-binding site comprises a third heavy-chain antibody variable domain (VHH3) comprising complementarity determining regions (CDRs) 1, 2, and 3, wherein the VHH3 CDR1 region comprises an amino acid sequence that is at least 80% identical to a selected VHH3 CDR1 amino acid sequence, the VHH3 CDR2 region comprises an amino acid sequence that is at least 80% identical to a selected VHH3 CDR2 amino acid sequence, and the VHH3 CDR3 region comprises an amino acid sequence that is at least 80% identical to a selected VHH3 CDR3 amino acid sequence;
wherein the selected VHH3 CDRs 1, 2, and 3 amino acid sequences are one of the following:
(1) the selected VHH3 CDRs 1, 2, 3 amino acid sequences are set forth in SEQ ID NOs: 1, 2, and 3, respectively;
(2) the selected VHH3 CDRs 1, 2, 3 amino acid sequences are set forth in SEQ ID NOs: 4, 5, and 6, respectively;
(3) the selected VHH3 CDRs 1, 2, 3 amino acid sequences are set forth in SEQ ID NOs: 7, 8, and 9, respectively;
(4) the selected VHH3 CDRs 1, 2, 3 amino acid sequences are set forth in SEQ ID NOs: 10, 11, and 12, respectively; and
(5) the selected VHH3 CDRs 1, 2, 3 amino acid sequences are set forth in SEQ ID NOs: 13, 14, and 15, respectively.

31. The multi-specific antibody or antigen-binding fragment thereof of claim 30, wherein the VHH3 comprises an amino acid sequence that is at least 80% identical to a selected VHH3 sequence, wherein the selected VHH3 sequence is selected from the group consisting of SEQ ID NOs: 22-26.

32. A multi-specific antibody or antigen-binding fragment thereof, comprising a first antigen-binding site that specifically binds to PD-L1, and a second antigen-binding site that specifically binds to LAG3.

33. The multi-specific antibody or antigen-binding fragment thereof of claim 32, wherein the first antigen-binding site comprises a first heavy-chain antibody variable domain (VHH1) that specifically binds to PD-L1, wherein
the VHH1 comprises complementarity determining regions (CDRs) 1, 2, and 3, wherein the VHH1 CDR1 region comprises an amino acid sequence that is at least 80% identical to SEQ ID NO: 19, the VHH1 CDR2 region comprises an amino acid sequence that is at least 80% identical to SEQ ID NO: 20, and the VHH1 CDR3 region comprises an amino acid sequence that is at least 80% identical to SEQ ID NO: 21.

34. The multi-specific antibody or antigen-binding fragment thereof of claim 33, wherein the VHH1 comprises CDRs 1, 2, 3 with the amino acid sequences set forth in SEQ ID NOs: 19, 20, and 21, respectively.

35. The multi-specific antibody or antigen-binding fragment thereof of claim 34, wherein the VHH1 comprises an amino acid sequence that is at least 80% identical to SEQ ID NO: 28.

36. The multi-specific antibody or antigen-binding fragment thereof of any one of claims 32-35, wherein the second antigen-binding site specifically binds to LAG3, and the second antigen-binding site comprises a second heavy-chain antibody variable domain (VHH2) comprising complementarity determining regions (CDRs) 1, 2, and 3, wherein the VHH2 CDR1 region comprises an amino acid sequence that is at least 80% identical to a selected VHH2 CDR1 amino acid sequence, the VHH2 CDR2 region comprises an amino acid sequence that is at least 80% identical to a selected VHH2 CDR2 amino acid sequence, and the VHH2 CDR3 region comprises an amino acid sequence that is at least 80% identical to a selected VHH2 CDR3 amino acid sequence;
wherein the selected VHH2 CDRs 1, 2, and 3 amino acid sequences are one of the following:
(1) the selected VHH2 CDRs 1, 2, 3 amino acid sequences are set forth in SEQ ID NOs: 1, 2, and 3, respectively;
(2) the selected VHH2 CDRs 1, 2, 3 amino acid sequences are set forth in SEQ ID NOs: 4, 5, and 6, respectively;
(3) the selected VHH2 CDRs 1, 2, 3 amino acid sequences are set forth in SEQ ID NOs: 7, 8, and 9, respectively;
(4) the selected VHH2 CDRs 1, 2, 3 amino acid sequences are set forth in SEQ ID NOs: 10, 11, and 12, respectively; and
(5) the selected VHH2 CDRs 1, 2, 3 amino acid sequences are set forth in SEQ ID NOs: 13, 14, and 15, respectively.

37. The multi-specific antibody or antigen-binding fragment thereof of claim 36, wherein the VHH2 comprises an amino acid sequence that is at least 80% identical to a selected VHH2 sequence, wherein the selected VHH2 sequence is selected from the group consisting of SEQ ID NOs: 22-26.

38. The multi-specific antibody or antigen-binding fragment thereof of any one of claims 32-37, further comprising a third antigen-binding site that specifically binds to TIGIT.

39. The multi-specific antibody or antigen-binding fragment thereof of any one of claims 32-38, wherein the third antigen-binding site specifically binds to TIGIT, and the third antigen-binding site comprises a third heavy-chain antibody variable domain (VHH3) that specifically binds to TIGIT, wherein
the VHH3 comprises complementarity determining regions (CDRs) 1, 2, and 3, wherein the VHH3 CDR1 region comprises an amino acid sequence that is at least 80% identical to SEQ ID NO: 16, the VHH3 CDR2 region comprises an amino acid sequence that is at least 80% identical to SEQ ID NO: 17, and the VHH3 CDR3 region comprises an amino acid sequence that is at least 80% identical to SEQ ID NO: 18.

40. The multi-specific antibody or antigen-binding fragment thereof of claim 39, wherein the VHH3 comprises an amino acid sequence that is at least 80% identical to SEQ ID NO: 27.

41. The multi-specific antibody or antigen-binding fragment thereof of any one of claims 23-40, wherein the VHH1, VHH2, and/or VHH3 are linked by a linker peptide sequence.

42. The multi-specific antibody or antigen-binding fragment thereof of claim 41, wherein the linker peptide sequence comprises a sequence that is at least 80% identical to any one of SEQ ID NOs: 30-35.

43. A polypeptide complex, comprising
(a) a first polypeptide comprising from N-terminus to C-terminus: a first heavy-chain antibody variable domain (VHH1), a first linker peptide sequence, a second heavy-chain antibody variable domain (VHH2), and a first Fc region (optionally including a hinge region);
(b) a second polypeptide comprising from N-terminus to C-terminus: a third heavy-chain antibody variable domain (VHH3), a second linker peptide sequence, a fourth heavy-chain antibody variable domain (VHH4), and a second Fc region (optionally including a hinge region); and
wherein the VHH1 and VHH3 specifically bind to TIGIT, wherein the VHH2 and VHH4 specifically bind to PD-L1.

44. The polypeptide complex of claim 43, wherein the first Fc region and/or the second Fc region comprise a sequence that is at least 80% identical to SEQ ID NO: 29.

45. The polypeptide complex of claim 43 or 44, wherein the VHH1 and/or VHH3 comprise an amino acid sequence that is at least 90% identical to SEQ ID NO: 27; and wherein the VHH2 and/or VHH4 comprise an amino acid sequence that is at least 90% identical to SEQ ID NO: 28.

46. The polypeptide complex of any one of claims 43-45, wherein the first polypeptide further comprises a fifth heavy-chain antibody variable domain (VHH5) that specifically binds to LAG3, wherein the VHH5 is linked to the N-terminus of the VHH1 via a third linker peptide sequence.

47. The polypeptide complex of claim 46, wherein the VHH5 comprises an amino acid sequence that is at least 90% identical to SEQ ID NO: 22, 23, 24, 25, or 26.

48. The polypeptide complex of any one of claims 43-47, wherein the first, second, and/or third linker peptide sequence comprises a sequence that is at least 80% identical to any one of SEQ ID NOs: 30-35.

49. A polypeptide complex, comprising
(a) a first polypeptide comprising from N-terminus to C-terminus: a first heavy-chain antibody variable domain (VHH1), a first linker peptide sequence, a second heavy-chain antibody variable domain (VHH2), and a first Fc region (optionally including a hinge region);
(b) a second polypeptide comprising from N-terminus to C-terminus: a third heavy-chain antibody variable domain (VHH3), a second linker peptide sequence, a fourth heavy-chain antibody variable domain (VHH4), and a second Fc region (optionally including a hinge region); and
wherein the VHH1 and VHH3 specifically bind to LAG3, wherein the VHH2 and VHH4 specifically bind to PD-L1.

50. The polypeptide complex of claim 49, wherein the first Fc region and/or the second Fc region comprise a sequence that is at least 80% identical to SEQ ID NO: 29.

51. The polypeptide complex of claim 49 or 50, wherein the VHH1 and/or VHH3 comprise an amino acid sequence that is at least 90% identical to SEQ ID NO: 22, 23, 24, 25, or 26; and wherein the VHH2 and/or VHH4 comprise an amino acid sequence that is at least 90% identical to SEQ ID NO: 28.

52. The polypeptide complex of any one of claims 49-51, wherein the first polypeptide further comprises a fifth heavy-chain antibody variable domain (VHH5) that specifically binds to TIGIT, wherein the VHH5 is linked to the N-terminus of the VHH1 via a third linker peptide sequence.

53. The polypeptide complex of claim 52, wherein the VHH5 comprises an amino acid sequence that is at least 90% identical to SEQ ID NO: 27.

54. The polypeptide complex of any one of claims 49-53, wherein the first, second, and/or third linker peptide sequence comprises a sequence that is at least 80% identical to any one of SEQ ID NOs: 30-35.

55. A nucleic acid comprising a polynucleotide encoding the antibody or antigen-binding fragment thereof of any one of claims 1-22, the multi-specific antibody or antigen-binding fragment thereof of any one of claims 23-42, or the polypeptide complex of any one of claims 43-54.

56. The nucleic acid of claim 55, wherein the nucleic acid is a DNA (e.g., cDNA) or RNA (e.g., mRNA).

57. A vector comprising one or more of the nucleic acids of claim 55 or 56.

58. A cell comprising the vector of claim 57.

59. The cell of claim 58, wherein the cell is a CHO cell.

60. A cell comprising one or more of the nucleic acid of claim 55 or 56.

61. A method of producing an antibody or an antigen-binding fragment thereof, the method comprising
(a) culturing the cell of any one of claims 58-60 under conditions sufficient for the cell to produce the antibody or the antigen-binding fragment; and
(b) collecting the antibody or the antigen-binding fragment produced by the cell.

62. An antibody-drug conjugate comprising the antibody or antigen-binding fragment thereof of any one of claims 1-22, the multi-specific antibody or antigen-binding fragment thereof of any one of claims 23-42, or the polypeptide complex of any one of claims 43-54, covalently bound to a therapeutic agent.

63. The antibody drug conjugate of claim 62, wherein the therapeutic agent is a cytotoxic or cytostatic agent.

64. A method of treating a subject having cancer, the method comprising administering a therapeutically effective amount of a composition comprising the antibody or antigen-binding fragment thereof of any one of claims 1-22, the multi-specific antibody or antigen-binding fragment thereof of any one of claims 23-42, the polypeptide complex of any one of claims 43-54, or the antibody-drug conjugate of claim 62 or 63, to the subject.

65. The method of claim 64, wherein the subject has a cancer expressing CD155, MHC class II molecules and/or FGL1 (Fibrinogen-like protein 1).

66. The method of claim 64 or 65, wherein the subject has a cancer expressing PD-L1.

67. The method of any one of claims 64-66, wherein the cancer is liver cancer melanoma, non-small cell lung cancer, head and neck squamous cell carcinoma, relapsed or refractory classical Hodgkin lymphoma, squamous cell lung cancer, renal cell carcinoma, pancreatic cancer, breast cancer, lung cancer, or cutaneous squamous cell carcinoma.

68. A method of decreasing the rate of tumor growth, the method comprising
contacting a tumor cell with an effective amount of a composition comprising the antibody or antigen-binding fragment thereof of any one of claims 1-22, the multi-specific antibody or antigen-binding fragment thereof of any one of claims 23-42, the polypeptide complex of any one of claims 43-54, or the antibody-drug conjugate of claim 62 or 63.

69. A method of killing a tumor cell, the method comprising
contacting a tumor cell with an effective amount of a composition comprising the antibody or antigen-binding fragment thereof of any one of claims 1-22, the multi-specific antibody or antigen-binding fragment thereof of any one of claims 23-42, the polypeptide complex of any one of claims 43-54, or the antibody-drug conjugate of claim 62 or 63.

70. A method of killing a tumor cell, the method comprising
contacting a tumor cell with an effective amount of a composition comprising the antibody or antigen-binding fragment thereof of any one of claims 1-22, the multi-specific antibody or antigen-binding fragment thereof of any one of claims 23-42, the polypeptide complex of any one of claims 43-54, or the antibody-drug conjugate of claim 62 or 63.

71. A pharmaceutical composition comprising the antibody or antigen-binding fragment thereof of any one of claims 1-22, the multi-specific antibody or antigen-binding fragment thereof of any one of claims 23-42, the polypeptide complex of any one of claims 43-54, and a pharmaceutically acceptable carrier.

72. A pharmaceutical composition comprising the antibody-drug conjugate of claim 62 or 63, and a pharmaceutically acceptable carrier.


SEQUENCE LISTING (39 sequences)

SEQ ID NO: 1 - protein, 10 aa, Synthetic construct
GFTLDDLDIG

SEQ ID NO: 2 - protein, 17 aa, Synthetic construct
AISSWDGSTYYADSVKG

SEQ ID NO: 3 - protein, 18 aa, Synthetic construct
AAGLVSGYGSSWSLQTDY

SEQ ID NO: 4 - protein, 10 aa, Synthetic construct
GLTFSRHSMG

SEQ ID NO: 5 - protein, 17 aa, Synthetic construct
AIRWDGTRTYYADSVSG

SEQ ID NO: 6 - protein, 17 aa, Synthetic construct
GSYLYGSALLSITEYNY

SEQ ID NO: 7 - protein, 10 aa, Synthetic construct
GLPFGDYAIG

SEQ ID NO: 8 - protein, 16 aa, Synthetic construct
CITNNGSTYYADSVKG

SEQ ID NO: 9 - protein, 13 aa, Synthetic construct
SGSYRYTPYEYDY

SEQ ID NO: 10 - protein, 10 aa, Synthetic construct
GLPFGDYAIG

SEQ ID NO: 11 - protein, 16 aa, Synthetic construct
CITNSGSTYYADSVKG

SEQ ID NO: 12 - protein, 13 aa, Synthetic construct
SGSYRYTPYEYDY

SEQ ID NO: 13 - protein, 10 aa, Synthetic construct
GLPFWDYAIG

SEQ ID NO: 14 - protein, 16 aa, Synthetic construct
CITNSGSTYYADSVKG

SEQ ID NO: 15 - protein, 13 aa, Synthetic construct
SGSYRYTPYEYDY

SEQ ID NO: 16 - protein, 10 aa, Synthetic construct
SRTFSRYAMG

SEQ ID NO: 17 - protein, 17 aa, Synthetic construct
SISWSGGGLEYAESVKG

SEQ ID NO: 18 - protein, 17 aa, Synthetic construct
AADPWGSLVNGAVRYEY

SEQ ID NO: 19 - protein, 10 aa, Synthetic construct
GKMSSRRCMA

SEQ ID NO: 20 - protein, 17 aa, Synthetic construct
KLLTTSGSTYLADSVKG

SEQ ID NO: 21 - protein, 11 aa, Synthetic construct
TLVTSSGAFQY

SEQ ID NO: 22 - protein, 125 aa, Synthetic construct
QVQLVESGGGLVQAGGSLRLSCAVSGFTLDDLDIGWFRQAPGKEREGVSAISSWDGSTYYADSVKGRFTISSDNGKNTVYLQMNSLKPEDTAVYYCAAGLVSGYGSSWSLQTDYWGQGTLVTVSS

SEQ ID NO: 23 - protein, 124 aa, Synthetic construct
QVQLVESGGGLVQAGGSLRLSCSTSGLTFSRHSMGWSRQAPGKEREFVSAIRWDGTRTYYADSVSGRFAISRDDANKIVYLQMNDLKPDDTAVYYCGSYLYGSALLSITEYNYWGQGTLVTVSS

SEQ ID NO: 24 - protein, 127 aa, Synthetic construct
QVQLVESGGGLVQAGGSLRLSCAASGLPFGDYAIGWYRQAPGKEREGVSCITNNGSTYYADSVKGRFTISSDIAKNTVFLQMNSLKPEDTAVYSCAANGWGDCSGSYRYTPYEYDYWGQGTLVTVSS

SEQ ID NO: 25 - protein, 127 aa, Synthetic construct
QVQLVESGGGLVQAGGSLRLSCAASGLPFGDYAIGWYRQAPGKEREGVSCITNSGSTYYADSVKGRFTISSDIAKNTVFLQMNSLKPEDTAVYSCAANGWGDCSGSYRYTPYEYDYWGQGTLVTVSS

SEQ ID NO: 26 - protein, 127 aa, Synthetic construct
QVQLVESGGGLVQAGGSLRLSCAASGLPFWDYAIGWYRQAPGKEREGVSCITNSGSTYYADSVKGRFTISSDIAKNTVFLQMNSLKPEDTAVYSCAANGWGDCSGSYRYTPYEYDYWGQGTLVTVSS

SEQ ID NO: 27 - protein, 124 aa, Synthetic construct
EVQLVESGGGLVQPGGSLRLSCAASSRTFSRYAMGWFRRAPGKEREFVASISWSGGGLEYAESVKGRFTISRDGSKNTVYLQMNSLAAEDTAVYYCAADPWGSLVNGAVRYEYWGQGTLVTVSS

SEQ ID NO: 28 - protein, 128 aa, Synthetic construct
QVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSS

SEQ ID NO: 29 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 5 aa, Synthetic construct
GGGGS

SEQ ID NO: 31 - protein, 10 aa, Synthetic construct
GGGGSGGGGS

SEQ ID NO: 32 - protein, 15 aa, Synthetic construct
GGGGSGGGGSGGGGS

SEQ ID NO: 33 - protein, 20 aa, Synthetic construct
GGGGSGGGGSGGGGSGGGGS

SEQ ID NO: 34 - protein, 25 aa, Synthetic construct
GGGGSGGGGSGGGGSGGGGSGGGGS

SEQ ID NO: 35 - protein, 30 aa, Synthetic construct
GGGGSGGGGSGGGGSGGGGSGGGGSGGGGS

SEQ ID NO: 36 - protein, 626 aa, Synthetic construct
EVQLVESGGGLVQPGGSLRLSCAASSRTFSRYAMGWFRRAPGKEREFVASISWSGGGLEYAESVKGRFTISRDGSKNTVYLQMNSLAAEDTAVYYCAADPWGSLVNGAVRYEYWGQGTLVTVSSGGGGSGGGGSQVQLVESGGGLVQAGGSLRLSCAASGLPFWDYAIGWYRQAPGKEREGVSCITNSGSTYYADSVKGRFTISSDIAKNTVFLQMNSLKPEDTAVYSCAANGWGDCSGSYRYTPYEYDYWGQGTLVTVSSGGGGSQVQLQESGGGLVQPGGSLRLSCAASGKMSSRRCMAWFRQAPGKERERVAKLLTTSGSTYLADSVKGRFTISQNNAKSTVYLQMNSLKPEDTAMYYCAADSFEDPTCTLVTSSGAFQYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 15 aa, Synthetic construct
DPWGSLVNGAVRYEY

SEQ ID NO: 38 - protein, 19 aa, Synthetic construct
DSFEDPTCTLVTSSGAFQY

SEQ ID NO: 39 - protein, 19 aa, Synthetic construct
NGWGDCSGSYRYTPYEYDY